SEQUENCE LISTING (211 sequences)

SEQ ID NO: 1 - protein, 6 aa, Homo sapiens
IYYSSI

SEQ ID NO: 2 - protein, 10 aa, Homo sapiens
SIYPYYGSTS

SEQ ID NO: 3 - protein, 9 aa, Homo sapiens
KWYYGDVGM

SEQ ID NO: 4 - protein, 5 aa, Homo sapiens
SVSSA

SEQ ID NO: 5 - protein, 7 aa, Homo sapiens
SASSLYS

SEQ ID NO: 6 - protein, 6 aa, Homo sapiens
SEYRLI

SEQ ID NO: 7 - protein, 231 aa, Homo sapiens
EISEVQLVESGGGLVQPGGSLRLSCAASGFNIYYSSIHWVRQAPGKGLEWVASIYPYYGSTSYADSVKGRFTISADTSKNTAYLQMNSLRAEDTAVYYCARKWYYGDVGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 8 - protein, 215 aa, Homo sapiens
SDIQMTQSPSSLSASVGDRVTITCRASQSVSSAVAWYQQKPGKAPKLLIYSASSLYSGVPSRFSGSRSGTDFTLTISSLQPEDFATYYCQQSEYRLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 31 aa, Homo sapiens
EISEVQLVESGGGLVQPGGSLRLSCAASGFN

SEQ ID NO: 10 - protein, 15 aa, Homo sapiens
HWVRQAPGKGLEWVA

SEQ ID NO: 11 - protein, 39 aa, Homo sapiens
YADSVKGRFTISADTSKNTAYLQMNSLRAEDTAVYYCAR

SEQ ID NO: 12 - protein, 121 aa, Homo sapiens
DYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 13 - protein, 28 aa, Homo sapiens
SDIQMTQSPSSLSASVGDRVTITCRASQ

SEQ ID NO: 14 - protein, 17 aa, Homo sapiens
VAWYQQKPGKAPKLLIY

SEQ ID NO: 15 - protein, 34 aa, Homo sapiens
GVPSRFSGSRSGTDFTLTISSLQPEDFATYYCQQ

SEQ ID NO: 16 - protein, 118 aa, Homo sapiens
TFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 6 aa, Homo sapiens
LSSSYI

SEQ ID NO: 18 - protein, 10 aa, Homo sapiens
YIYSSSGSTY

SEQ ID NO: 19 - protein, 18 aa, Homo sapiens
WPTYYYWLVGWMYDVGAF

SEQ ID NO: 20 - protein, 7 aa, Homo sapiens
SSSEYPV

SEQ ID NO: 21 - protein, 240 aa, Homo sapiens
EISEVQLVESGGGLVQPGGSLRLSCAASGFNLSSSYIHWVRQAPGKGLEWVAYIYSSSGSTYYADSVKGRFTISADTSKNTAYLQMNSLRAEDTAVYYCARWPTYYYWLVGWMYDVGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 22 - protein, 216 aa, Homo sapiens
SDIQMTQSPSSLSASVGDRVTITCRASQSVSSAVAWYQQKPGKAPKLLIYSASSLYSGVPSRFSGSRSGTDFTLTISSLQPEDFATYYCQQSSSEYPVTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 6 aa, Homo sapiens
FSYSYI

SEQ ID NO: 24 - protein, 10 aa, Homo sapiens
YISSSSGYTY

SEQ ID NO: 25 - protein, 18 aa, Homo sapiens
SGLYSHGVWQNWYGYMGF

SEQ ID NO: 26 - protein, 6 aa, Homo sapiens
SLQWPI

SEQ ID NO: 27 - protein, 240 aa, Homo sapiens
EISEVQLVESGGGLVQPGGSLRLSCAASGFNFSYSYIHWVRQAPGKGLEWVAYISSSSGYTYYADSVKGRFTISADTSKNTAYLQMNSLRAEDTAVYYCARSGLYSHGVWQNWYGYMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 28 - protein, 215 aa, Homo sapiens
SDIQMTQSPSSLSASVGDRVTITCRASQSVSSAVAWYQQKPGKAPKLLIYSASSLYSGVPSRFSGSRSGTDFTLTISSLQPEDFATYYCQQSLQWPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 6 aa, Homo sapiens
VYSYSI

SEQ ID NO: 30 - protein, 10 aa, Homo sapiens
SIYSSYSYTS

SEQ ID NO: 31 - protein, 12 aa, Homo sapiens
KYHFKPTYWGAM

SEQ ID NO: 32 - protein, 7 aa, Homo sapiens
SSLYYPL

SEQ ID NO: 33 - protein, 234 aa, Homo sapiens
EISEVQLVESGGGLVQPGGSLRLSCAASGFNVYSYSIHWVRQAPGKGLEWVASIYSSYSYTSYADSVKGRFTISADTSKNTAYLQMNSLRAEDTAVYYCARKYHFKPTYWGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 34 - protein, 216 aa, Homo sapiens
SDIQMTQSPSSLSASVGDRVTITCRASQSVSSAVAWYQQKPGKAPKLLIYSASSLYSGVPSRFSGSRSGTDFTLTISSLQPEDFATYYCQQSSLYYPLTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 6 aa, Homo sapiens
VYYSSI

SEQ ID NO: 36 - protein, 10 aa, Homo sapiens
SISPYYGSTS

SEQ ID NO: 37 - protein, 12 aa, Homo sapiens
RWYYTKVFMWGM

SEQ ID NO: 38 - protein, 6 aa, Homo sapiens
SSSSLI

SEQ ID NO: 39 - protein, 234 aa, Homo sapiens
EISEVQLVESGGGLVQPGGSLRLSCAASGFNVYYSSIHWVRQAPGKGLEWVASISPYYGSTSYADSVKGRFTISADTSKNTAYLQMNSLRAEDTAVYYCARRWYYTKVFMWGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 40 - protein, 215 aa, Homo sapiens
SDIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 6 aa, Homo sapiens
VSSYSI

SEQ ID NO: 42 - protein, 10 aa, Homo sapiens
SISSSSGSTS

SEQ ID NO: 43 - protein, 18 aa, Homo sapiens
ESNQYYYYYNVLWWYQAF

SEQ ID NO: 44 - protein, 240 aa, Homo sapiens
EISEVQLVESGGGLVQPGGSLRLSCAASGFNVSSYSIHWVRQAPGKGLEWVASISSSSGSTSYADSVKGRFTISADTSKNTAYLQMNSLRAEDTAVYYCARESNQYYYYYNVLWWYQAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 45 - protein, 6 aa, Homo sapiens
FYSSYI

SEQ ID NO: 46 - protein, 6 aa, Homo sapiens
RSGGGL

SEQ ID NO: 47 - protein, 228 aa, Homo sapiens
EISEVQLVESGGGLVQPGGSLRLSCAASGFNFYSSYIHWVRQAPGKGLEWVASISSSSGSTSYADSVKGRFTISADTSKNTAYLQMNSLRAEDTAVYYCARRSGGGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 48 - protein, 10 aa, Homo sapiens
SISSYYGSTY

SEQ ID NO: 49 - protein, 6 aa, Homo sapiens
WKWVAM

SEQ ID NO: 50 - protein, 6 aa, Homo sapiens
DLFMPI

SEQ ID NO: 51 - protein, 228 aa, Homo sapiens
EISEVQLVESGGGLVQPGGSLRLSCAASGFNVYYSSIHWVRQAPGKGLEWVASISSYYGSTYYADSVKGRFTISADTSKNTAYLQMNSLRAEDTAVYYCARWKWVAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 52 - protein, 215 aa, Homo sapiens
SDIQMTQSPSSLSASVGDRVTITCRASQSVSSAVAWYQQKPGKAPKLLIYSASSLYSGVPSRFSGSRSGTDFTLTISSLQPEDFATYYCQQDLFMPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 6 aa, Homo sapiens
FSSSSI

SEQ ID NO: 54 - protein, 10 aa, Homo sapiens
SIYSYSGSTY

SEQ ID NO: 55 - protein, 20 aa, Homo sapiens
GLYYYLPSYYIYYRSYKFGM

SEQ ID NO: 56 - protein, 242 aa, Homo sapiens
EISEVQLVESGGGLVQPGGSLRLSCAASGFNFSSSSIHWVRQAPGKGLEWVASIYSYSGSTYYADSVKGRFTISADTSKNTAYLQMNSLRAEDTAVYYCARGLYYYLPSYYIYYRSYKFGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 57 - protein, 6 aa, Homo sapiens
FSYSSI

SEQ ID NO: 58 - protein, 10 aa, Homo sapiens
SIYPYSGSTY

SEQ ID NO: 59 - protein, 12 aa, Homo sapiens
KQVSGWWWYYGF

SEQ ID NO: 60 - protein, 6 aa, Homo sapiens
SYWWPI

SEQ ID NO: 61 - protein, 234 aa, Homo sapiens
EISEVQLVESGGGLVQPGGSLRLSCAASGFNFSYSSIHWVRQAPGKGLEWVASIYPYSGSTYYADSVKGRFTISADTSKNTAYLQMNSLRAEDTAVYYCARKQVSGWWWYYGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 62 - protein, 215 aa, Homo sapiens
SDIQMTQSPSSLSASVGDRVTITCRASQSVSSAVAWYQQKPGKAPKLLIYSASSLYSGVPSRFSGSRSGTDFTLTISSLQPEDFATYYCQQSYWWPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 10 aa, Homo sapiens
SIYSSSSSTY

SEQ ID NO: 64 - protein, 16 aa, Homo sapiens
RYYHYRYPLFWGWYGM

SEQ ID NO: 65 - protein, 6 aa, Homo sapiens
SFWWPI

SEQ ID NO: 66 - protein, 238 aa, Homo sapiens
EISEVQLVESGGGLVQPGGSLRLSCAASGFNVSSYSIHWVRQAPGKGLEWVASIYSSSSSTYYADSVKGRFTISADTSKNTAYLQMNSLRAEDTAVYYCARRYYHYRYPLFWGWYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 67 - protein, 215 aa, Homo sapiens
SDIQMTQSPSSLSASVGDRVTITCRASQSVSSAVAWYQQKPGKAPKLLIYSASSLYSGVPSRFSGSRSGTDFTLTISSLQPEDFATYYCQQSFWWPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 10 aa, Homo sapiens
SIYSYSGYTY

SEQ ID NO: 69 - protein, 12 aa, Homo sapiens
STYPHVYLYFGL

SEQ ID NO: 70 - protein, 6 aa, Homo sapiens
SYYWPV

SEQ ID NO: 71 - protein, 234 aa, Homo sapiens
EISEVQLVESGGGLVQPGGSLRLSCAASGFNVYSYSIHWVRQAPGKGLEWVASIYSYSGYTYYADSVKGRFTISADTSKNTAYLQMNSLRAEDTAVYYCARSTYPHVYLYFGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 72 - protein, 215 aa, Homo sapiens
SDIQMTQSPSSLSASVGDRVTITCRASQSVSSAVAWYQQKPGKAPKLLIYSASSLYSGVPSRFSGSRSGTDFTLTISSLQPEDFATYYCQQSYYWPVTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 6 aa, Homo sapiens
ISSSSI

SEQ ID NO: 74 - protein, 10 aa, Homo sapiens
YISSYSGSTY

SEQ ID NO: 75 - protein, 14 aa, Homo sapiens
YYHSPLWYYSYSGM

SEQ ID NO: 76 - protein, 7 aa, Homo sapiens
DYSWSPI

SEQ ID NO: 77 - protein, 236 aa, Homo sapiens
EISEVQLVESGGGLVQPGGSLRLSCAASGFNISSSSIHWVRQAPGKGLEWVAYISSYSGSTYYADSVKGRFTISADTSKNTAYLQMNSLRAEDTAVYYCARYYHSPLWYYSYSGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 78 - protein, 216 aa, Homo sapiens
SDIQMTQSPSSLSASVGDRVTITCRASQSVSSAVAWYQQKPGKAPKLLIYSASSLYSGVPSRFSGSRSGTDFTLTISSLQPEDFATYYCQQDYSWSPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 10 aa, Homo sapiens
SISSYYSYTY

SEQ ID NO: 80 - protein, 17 aa, Homo sapiens
GAWYGYLGYYAYYYYAL

SEQ ID NO: 81 - protein, 7 aa, Homo sapiens
MGYETLI

SEQ ID NO: 82 - protein, 239 aa, Homo sapiens
EISEVQLVESGGGLVQPGGSLRLSCAASGFNISSSSIHWVRQAPGKGLEWVASISSYYSYTYYADSVKGRFTISADTSKNTAYLQMNSLRAEDTAVYYCARGAWYGYLGYYAYYYYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 83 - protein, 216 aa, Homo sapiens
SDIQMTQSPSSLSASVGDRVTITCRASQSVSSAVAWYQQKPGKAPKLLIYSASSLYSGVPSRFSGSRSGTDFTLTISSLQPEDFATYYCQQMGYETLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 19 aa, Homo sapiens
ESSQTKKYWYWVVQWYTAM

SEQ ID NO: 85 - protein, 6 aa, Homo sapiens
YSQWLI

SEQ ID NO: 86 - protein, 241 aa, Homo sapiens
EISEVQLVESGGGLVQPGGSLRLSCAASGFNFSSSSIHWVRQAPGKGLEWVASISSSSGSTSYADSVKGRFTISADTSKNTAYLQMNSLRAEDTAVYYCARESSQTKKYWYWVVQWYTAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 87 - protein, 215 aa, Homo sapiens
SDIQMTQSPSSLSASVGDRVTITCRASQSVSSAVAWYQQKPGKAPKLLIYSASSLYSGVPSRFSGSRSGTDFTLTISSLQPEDFATYYCQQYSQWLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 10 aa, Homo sapiens
SISPYYGYTS

SEQ ID NO: 89 - protein, 15 aa, Homo sapiens
HYYYGFSWFSSGYAM

SEQ ID NO: 90 - protein, 6 aa, Homo sapiens
GSSSPI

SEQ ID NO: 91 - protein, 237 aa, Homo sapiens
EISEVQLVESGGGLVQPGGSLRLSCAASGFNVSSYSIHWVRQAPGKGLEWVASISPYYGYTSYADSVKGRFTISADTSKNTAYLQMNSLRAEDTAVYYCARHYYYGFSWFSSGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 92 - protein, 215 aa, Homo sapiens
SDIQMTQSPSSLSASVGDRVTITCRASQSVSSAVAWYQQKPGKAPKLLIYSASSLYSGVPSRFSGSRSGTDFTLTISSLQPEDFATYYCQQGSSSPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - DNA, 255 bp, Homo sapiens
  source 1..255: mol_type = other DNA
gagatctccgaggttcagctggtggagtctggcggtggcctggtgcagccagggggctcactccgtttgtcctgtgcagcttctggcttcaacatctattattcttctatacactgggtgcgtcaggccccgggtaagggcctggaatgggttgcatctatttatccttattatggctctacttcttatgccgatagcgtcaagggccgtttcactataagcgcagacacatccaaaaacacagcctacctacaa

SEQ ID NO: 94 - DNA, 255 bp, Homo sapiens
  source 1..255: mol_type = other DNA
tccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgca

SEQ ID NO: 95 - DNA, 255 bp, Homo sapiens
  source 1..255: mol_type = other DNA
gagatctccgaggttcagctggtggagtctggcggtggcctggtgcagccagggggctcactccgtttgtcctgtgcagcttctggcttcaacctctcttcttcttatatacactgggtgcgtcaggccccgggtaagggcctggaatgggttgcatatatttattcttcttctggctctacttattatgccgatagcgtcaagggccgtttcactataagcgcagacacatccaaaaacacagcctacctacaa

SEQ ID NO: 96 - DNA, 255 bp, Homo sapiens; identical to SEQ ID NO: 94
  source 1..255: mol_type = other DNA

SEQ ID NO: 97 - DNA, 255 bp, Homo sapiens
  source 1..255: mol_type = other DNA
gagatctccgaggttcagctggtggagtctggcggtggcctggtgcagccagggggctcactccgtttgtcctgtgcagcttctggcttcaacttctcttattcttatatacactgggtgcgtcaggccccgggtaagggcctggaatgggttgcatatatttcttcttcttctggctatacttattatgccgatagcgtcaagggccgtttcactataagcgcagacacatccaaaaacacagcctacctacaa

SEQ ID NO: 98 - DNA, 255 bp, Homo sapiens; identical to SEQ ID NO: 94
  source 1..255: mol_type = other DNA

SEQ ID NO: 99 - DNA, 255 bp, Homo sapiens
  source 1..255: mol_type = other DNA
gagatctccgaggttcagctggtggagtctggcggtggcctggtgcagccagggggctcactccgtttgtcctgtgcagcttctggcttcaacgtctattcttattctatacactgggtgcgtcaggccccgggtaagggcctggaatgggttgcatctatttattcttcttatagctatacttcttatgccgatagcgtcaagggccgtttcactataagcgcagacacatccaaaaacacagcctacctacaa

SEQ ID NO: 100 - DNA, 255 bp, Homo sapiens; identical to SEQ ID NO: 94
  source 1..255: mol_type = other DNA

SEQ ID NO: 101 - DNA, 255 bp, Homo sapiens
  source 1..255: mol_type = other DNA
gagatctccgaggttcagctggtggagtctggcggtggcctggtgcagccagggggctcactccgtttgtcctgtgcagcttctggcttcaacgtctattattcttctatacactgggtgcgtcaggccccgggtaagggcctggaatgggttgcatctatttctccttattatggctctacttcttatgccgatagcgtcaagggccgtttcactataagcgcagacacatccaaaaacacagcctacctacaa

SEQ ID NO: 102 - DNA, 255 bp, Homo sapiens; identical to SEQ ID NO: 94
  source 1..255: mol_type = other DNA

SEQ ID NO: 103 - DNA, 255 bp, Homo sapiens
  source 1..255: mol_type = other DNA
gagatctccgaggttcagctggtggagtctggcggtggcctggtgcagccagggggctcactccgtttgtcctgtgcagcttctggcttcaacgtctcttcttattctatacactgggtgcgtcaggccccgggtaagggcctggaatgggttgcatctatttcttcttcttctggctctacttcttatgccgatagcgtcaagggccgtttcactataagcgcagacacatccaaaaacacagcctacctacaa

SEQ ID NO: 104 - DNA, 255 bp, Homo sapiens
  source 1..255: mol_type = other DNA
gagatctccgaggttcagctggtggagtctggcggtggcctggtgcagccagggggctcactccgtttgtcctgtgcagcttctggcttcaacttctattcttcttatatacactgggtgcgtcaggccccgggtaagggcctggaatgggttgcatctatttcttcttcttctggctctacttcttatgccgatagcgtcaagggccgtttcactataagcgcagacacatccaaaaacacagcctacctacaa

SEQ ID NO: 105 - DNA, 255 bp, Homo sapiens
  source 1..255: mol_type = other DNA
gagatctccgaggttcagctggtggagtctggcggtggcctggtgcagccagggggctcactccgtttgtcctgtgcagcttctggcttcaacgtctattattcttctatacactgggtgcgtcaggccccgggtaagggcctggaatgggttgcatctatttcttcttattatggctctacttattatgccgatagcgtcaagggccgtttcactataagcgcagacacatccaaaaacacagcctacctacaa

SEQ ID NO: 106 - DNA, 255 bp, Homo sapiens; identical to SEQ ID NO: 94
  source 1..255: mol_type = other DNA

SEQ ID NO: 107 - DNA, 255 bp, Homo sapiens
  source 1..255: mol_type = other DNA
gagatctccgaggttcagctggtggagtctggcggtggcctggtgcagccagggggctcactccgtttgtcctgtgcagcttctggcttcaacttttcttcttcttctatacactgggtgcgtcaggccccgggtaagggcctggaatgggttgcatctatttattcttattctggctctacttattatgccgatagcgtcaagggccgtttcactataagcgcagacacatccaaaaacacagcctacctacaa

SEQ ID NO: 108 - DNA, 255 bp, Homo sapiens
  source 1..255: mol_type = other DNA
gagatctccgaggttcagctggtggagtctggcggtggcctggtgcagccagggggctcactccgtttgtcctgtgcagcttctggcttcaacttctcttattcttctatacactgggtgcgtcaggccccgggtaagggcctggaatgggttgcatctatttatccttattctggctctacttattatgccgatagcgtcaagggccgtttcactataagcgcagacacatccaaaaacacagcctacctacaa

SEQ ID NO: 109 - DNA, 255 bp, Homo sapiens; identical to SEQ ID NO: 94
  source 1..255: mol_type = other DNA

SEQ ID NO: 110 - DNA, 255 bp, Homo sapiens
  source 1..255: mol_type = other DNA
gagatctccgaggttcagctggtggagtctggcggtggcctggtgcagccagggggctcactccgtttgtcctgtgcagcttctggcttcaacgtctcttcttattctatacactgggtgcgtcaggccccgggtaagggcctggaatgggttgcatctatttattcttcttctagctctacttattatgccgatagcgtcaagggccgtttcactataagcgcagacacatccaaaaacacagcctacctacaa

SEQ ID NO: 111 - DNA, 255 bp, Homo sapiens; identical to SEQ ID NO: 94
  source 1..255: mol_type = other DNA

SEQ ID NO: 112 - DNA, 255 bp, Homo sapiens
  source 1..255: mol_type = other DNA
gagatctccgaggttcagctggtggagtctggcggtggcctggtgcagccagggggctcactccgtttgtcctgtgcagcttctggcttcaacgtctattcttattctatacactgggtgcgtcaggccccgggtaagggcctggaatgggttgcatctatttattcttattctggctatacttattatgccgatagcgtcaagggccgtttcactataagcgcagacacatccaaaaacacagcctacctacaa

SEQ ID NO: 113 - DNA, 255 bp, Homo sapiens; identical to SEQ ID NO: 94
  source 1..255: mol_type = other DNA

SEQ ID NO: 114 - DNA, 255 bp, Homo sapiens
  source 1..255: mol_type = other DNA
gagatctccgaggttcagctggtggagtctggcggtggcctggtgcagccagggggctcactccgtttgtcctgtgcagcttctggcttcaacatctcttcttcttctatacactgggtgcgtcaggccccgggtaagggcctggaatgggttgcatatatttcttcttattctggctctacttattatgccgatagcgtcaagggccgtttcactataagcgcagacacatccaaaaacacagcctacctacaa

SEQ ID NO: 115 - DNA, 255 bp, Homo sapiens; identical to SEQ ID NO: 94
  source 1..255: mol_type = other DNA

SEQ ID NO: 116 - DNA, 255 bp, Homo sapiens
  source 1..255: mol_type = other DNA
gagatctccgaggttcagctggtggagtctggcggtggcctggtgcagccagggggctcactccgtttgtcctgtgcagcttctggcttcaacatctcttcttcttctatacactgggtgcgtcaggccccgggtaagggcctggaatgggttgcatctatttcttcttattatagctatacttattatgccgatagcgtcaagggccgtttcactataagcgcagacacatccaaaaacacagcctacctacaa

SEQ ID NO: 117 - DNA, 255 bp, Homo sapiens; identical to SEQ ID NO: 94
  source 1..255: mol_type = other DNA

SEQ ID NO: 118 - DNA, 255 bp, Homo sapiens
  source 1..255: mol_type = other DNA
gagatctccgaggttcagctggtggagtctggcggtggcctggtgcagccagggggctcactccgtttgtcctgtgcagcttctggcttcaacttttcttcttcttctatacactgggtgcgtcaggccccgggtaagggcctggaatgggttgcatctatttcttcttcttctggctctacttcttatgccgatagcgtcaagggccgtttcactataagcgcagacacatccaaaaacacagcctacctacaa

SEQ ID NO: 119 - DNA, 255 bp, Homo sapiens; identical to SEQ ID NO: 94
  source 1..255: mol_type = other DNA

SEQ ID NO: 120 - DNA, 255 bp, Homo sapiens
  source 1..255: mol_type = other DNA
gagatctccgaggttcagctggtggagtctggcggtggcctggtgcagccagggggctcactccgtttgtcctgtgcagcttctggcttcaacgtctcttcttattctatacactgggtgcgtcaggccccgggtaagggcctggaatgggttgcatctatttctccttattatggctatacttcttatgccgatagcgtcaagggccgtttcactataagcgcagacacatccaaaaacacagcctacctacaa

SEQ ID NO: 121 - DNA, 255 bp, Homo sapiens; identical to SEQ ID NO: 94
  source 1..255: mol_type = other DNA

SEQ ID NO: 122 - protein, 105 aa, Homo sapiens
VAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - protein, 108 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 124 - protein, 231 aa, Homo sapiens; identical to SEQ ID NO: 7

SEQ ID NO: 125 - protein, 215 aa, Homo sapiens; identical to SEQ ID NO: 8

SEQ ID NO: 126 - protein, 240 aa, Homo sapiens; identical to SEQ ID NO: 21

SEQ ID NO: 127 - protein, 216 aa, Homo sapiens; identical to SEQ ID NO: 22

SEQ ID NO: 128 - protein, 240 aa, Homo sapiens; identical to SEQ ID NO: 27

SEQ ID NO: 129 - protein, 215 aa, Homo sapiens; identical to SEQ ID NO: 28

SEQ ID NO: 130 - protein, 234 aa, Homo sapiens; identical to SEQ ID NO: 33

SEQ ID NO: 131 - protein, 216 aa, Homo sapiens; identical to SEQ ID NO: 34

SEQ ID NO: 132 - protein, 234 aa, Homo sapiens; identical to SEQ ID NO: 39

SEQ ID NO: 133 - protein, 215 aa, Homo sapiens; identical to SEQ ID NO: 40

SEQ ID NO: 134 - protein, 240 aa, Homo sapiens; identical to SEQ ID NO: 44

SEQ ID NO: 135 - protein, 228 aa, Homo sapiens; identical to SEQ ID NO: 47

SEQ ID NO: 136 - protein, 228 aa, Homo sapiens; identical to SEQ ID NO: 51

SEQ ID NO: 137 - protein, 215 aa, Homo sapiens; identical to SEQ ID NO: 52

SEQ ID NO: 138 - protein, 242 aa, Homo sapiens; identical to SEQ ID NO: 56

SEQ ID NO: 139 - protein, 234 aa, Homo sapiens; identical to SEQ ID NO: 61

SEQ ID NO: 140 - protein, 215 aa, Homo sapiens; identical to SEQ ID NO: 62

SEQ ID NO: 141 - protein, 238 aa, Homo sapiens; identical to SEQ ID NO: 66

SEQ ID NO: 142 - protein, 215 aa, Homo sapiens; identical to SEQ ID NO: 67

SEQ ID NO: 143 - protein, 234 aa, Homo sapiens; identical to SEQ ID NO: 71

SEQ ID NO: 144 - protein, 215 aa, Homo sapiens; identical to SEQ ID NO: 72

SEQ ID NO: 145 - protein, 236 aa, Homo sapiens; identical to SEQ ID NO: 77

SEQ ID NO: 146 - protein, 216 aa, Homo sapiens; identical to SEQ ID NO: 78

SEQ ID NO: 147 - protein, 239 aa, Homo sapiens; identical to SEQ ID NO: 82

SEQ ID NO: 148 - protein, 216 aa, Homo sapiens; identical to SEQ ID NO: 83

SEQ ID NO: 149 - protein, 241 aa, Homo sapiens; identical to SEQ ID NO: 86

SEQ ID NO: 150 - protein, 215 aa, Homo sapiens; identical to SEQ ID NO: 87

SEQ ID NO: 151 - protein, 237 aa, Homo sapiens; identical to SEQ ID NO: 91

SEQ ID NO: 152 - protein, 215 aa, Homo sapiens; identical to SEQ ID NO: 92

SEQ ID NO: 153 - protein, 105 aa, Homo sapiens; identical to SEQ ID NO: 122

SEQ ID NO: 154 - protein, 108 aa, Homo sapiens; identical to SEQ ID NO: 123

SEQ ID NO: 155 - protein, 5 aa, Homo sapiens
DKTHT

SEQ ID NO: 156 - protein, 4 aa, Homo sapiens
CPPC

SEQ ID NO: 157 - protein, 15 aa, Homo sapiens
CPEPKSCDTPPPCPR

SEQ ID NO: 158 - protein, 12 aa, Homo sapiens
ELKTPLGDTTHT

SEQ ID NO: 159 - protein, 10 aa, Homo sapiens
KSCDKTHTCP

SEQ ID NO: 160 - protein, 7 aa, Homo sapiens
KCCVDCP

SEQ ID NO: 161 - protein, 7 aa, Homo sapiens
KYGPPCP

SEQ ID NO: 162 - protein, 15 aa, Homo sapiens
EPKSCDKTHTCPPCP

SEQ ID NO: 163 - protein, 17 aa, Homo sapiens
ELKTPLGDTTHTCPRCP

SEQ ID NO: 164 - protein, 12 aa, Homo sapiens
SPNMVPHAHHAQ

SEQ ID NO: 165 - protein, 12 aa, Homo sapiens
ESKYGPPCPPCP

SEQ ID NO: 166 - protein, 15 aa, Homo sapiens
EPKSCDKTYTCPPCP

SEQ ID NO: 167 - protein, 45 aa, Homo sapiens
TTTPAPRPPTPAPTIASQPLSLRPEACRPAAGGAVHTRGLDFACD

SEQ ID NO: 168 - protein, 110 aa, Homo sapiens
APEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAK

SEQ ID NO: 169 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 170 - protein, 27 aa, Homo sapiens
FWVLVVVGGVLACYSLLVTVAFIIFWV

SEQ ID NO: 171 - protein, 23 aa, Homo sapiens
LGLLVAGVLVLLVSLGVAIHLCC

SEQ ID NO: 172 - protein, 25 aa, Homo sapiens
ALIVLGGVAGLLLFIGLGIFFCVRC

SEQ ID NO: 173 - protein, 23 aa, Homo sapiens
LCYLLDGILFIYGVILTALFLRV

SEQ ID NO: 174 - protein, 26 aa, Homo sapiens
WVLVVVGGVLACYSLLVTVAFIIFWV

SEQ ID NO: 175 - protein, 26 aa, Homo sapiens
VAAILGLGLVLGLLGPLAILLALYLL

SEQ ID NO: 176 - protein, 24 aa, Homo sapiens
ALPAALAVISFLLGLGLGVACVLA

SEQ ID NO: 177 - protein, 113 aa, Homo sapiens
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 178 - protein, 112 aa, Homo sapiens
MGGLEPCSRLLLLPLLLAVSGLRPVQAQAQSDCSCSTVSPGVLAGIVMGDLVLTVLIALAVYFLGRLVPRGRGAAEATRKQRITETESPYQELQGQRSDVYSDLNTQRPYYK

SEQ ID NO: 179 - protein, 102 aa, Homo sapiens
MGGLEPCSRLLLLPLLLAVSDCSCSTVSPGVLAGIVMGDLVLTVLIALAVYFLGRLVPRGRGAAEAATRKQRITETESPYQELQGQRSDVYSDLNTQRPYYK

SEQ ID NO: 180 - protein, 101 aa, Homo sapiens
MGGLEPCSRLLLLPLLLAVSDCSCSTVSPGVLAGIVMGDLVLTVLIALAVYFLGRLVPRGRGAAEATRKQRITETESPYQELQGQRSDVYSDLNTQRPYYK

SEQ ID NO: 181 - protein, 86 aa, Homo sapiens
MIPAVVLLLLLLVEQAAALGEPQLCYILDAILFLYGIVLTLLYCRLKIQVRKAAITSYEKSDGVYTGLSTRNQETYETLKHEKPPQ

SEQ ID NO: 182 - protein, 21 aa, Homo sapiens
DGVYTGLSTRNQETYETLKHE

SEQ ID NO: 183 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 184 - protein, 127 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRTADTQALLRNDQVYQPLRDRDDAQYSHLGGNWARNK

SEQ ID NO: 185 - protein, 21 aa, Homo sapiens
DQVYQPLRDRDDAQYSHLGGN

SEQ ID NO: 186 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 187 - protein, 21 aa, Homo sapiens
NPDYEPIRKGQRDLYSGLNQR

SEQ ID NO: 188 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 189 - protein, 21 aa, Homo sapiens
DQLYQPLKDREDDQYSHLQGN

SEQ ID NO: 190 - protein, 163 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 191 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 192 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 193 - protein, 21 aa, Homo sapiens
NQLYNELNLGRREEYDVLDKR

SEQ ID NO: 194 - protein, 22 aa, Homo sapiens
EGLYNELQKDKMAEAYSEIGMK

SEQ ID NO: 195 - protein, 21 aa, Homo sapiens
DGLYQGLSTATKDTYDALHMQ

SEQ ID NO: 196 - protein, 226 aa, Homo sapiens
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 197 - protein, 188 aa, Homo sapiens
MPGGPGVLQALPATIFLLFLLSAVYLGPGCQALWMHKVPASLMVSLGEDAHFQCPHNSSNNANVTWWRVLHGNYTWPPEFLGPGEDPNEPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 198 - protein, 21 aa, Homo sapiens
ENLYEGLNLDDCSMYEDISRG

SEQ ID NO: 199 - protein, 68 aa, Homo sapiens
FWVLVVVGGVLACYSLLVTVAFIIFWVRSKRSRLLHSDYMNMTPRRPGPTRKHYQPYAPPRDFAAYRS

SEQ ID NO: 200 - protein, 9 aa, synthetic construct
YPYDVPDYA

SEQ ID NO: 201 - protein, 8 aa, synthetic construct
DYKDDDDK

SEQ ID NO: 202 - protein, 10 aa, synthetic construct
EQKLISEEDL

SEQ ID NO: 203 - protein, 5 aa, synthetic construct
GSGGS

SEQ ID NO: 204 - protein, 4 aa, synthetic construct
GGGS

SEQ ID NO: 205 - protein, 4 aa, synthetic construct
GGSG

SEQ ID NO: 206 - protein, 5 aa, synthetic construct
GGSGG

SEQ ID NO: 207 - protein, 5 aa, synthetic construct
GSGSG

SEQ ID NO: 208 - protein, 5 aa, synthetic construct
GSGGG

SEQ ID NO: 209 - protein, 5 aa, synthetic construct
GGGSG

SEQ ID NO: 210 - protein, 5 aa, synthetic construct
GSSSG

SEQ ID NO: 211 - protein, 5 aa, synthetic construct
EAAAK

(19) United States
(12) Patent Application Publication
(10) Pub. No.: US 2025/0127814 A1
(43) Pub. Date: Apr. 24, 2025

(54) BOREALIN TARGETING POLYPEPTIDES FOR DETECTION AND TREATMENT OF CANCER

(71) Applicant: THE UNIVERSITY OF CHICAGO, Chicago, IL (US)

(72) Inventors: Anthony KOSSIAKOFF, Chicago, IL (US); Annika SÄÄF, Chicago, IL (US); Marcin URA, Chicago, IL (US); Somnath MUKHERJEE, Chicago, IL (US)

(73) Assignee: THE UNIVERSITY OF CHICAGO, Chicago, IL (US)

(21) Appl. No.: 18/695,701

(22) PCT Filed: Oct. 6, 2022
(86) PCT No.: PCT/US2022/077720

§ 371 Date: Mar. 26, 2024

Related U.S. Application Data
(60) Provisional application No. 63/252,883, filed on Oct. 6, 2021.

(51) Int. Cl. A61K 35/17; A61K 40/11; A61K 40/15; A61K 40/31; A61K 40/42; A61K 45/06; A61P 35/00; C07K 16/28; C12N 5/0783; C12N 15/85; G01N 33/573; G01N 33/58

(52) U.S. Cl. CPC A61K 35/17; A61K 40/11; A61K 40/15; A61K 40/31; A61K 40/421; A61K 40/4239; A61K 45/06; A61P 35/00; C07K 16/2809; C07K 16/2869; C12N 5/0636; C12N 5/0646; C12N 15/85; G01N 33/573; G01N 33/58; A61K 2239/13; A61K 2239/17; A61K 2239/21; A61K 2239/22; A61K 2239/29; C07K 2317/24; C07K 2317/31; C07K 2317/35; C07K 2317/55; C07K 2317/565; C07K 2317/567; C07K 2317/622; C07K 2317/76; C07K 2317/92; G01N 2333/7051; G01N 2333/72

(57) ABSTRACT

Aspects of the present disclosure are directed to borealin-targeting polypeptides, including antibodies, antibody-drug conjugates, antibody fragments, antibody-like molecules, and chimeric receptors. Also disclosed herein are nucleic acids encoding for such borealin-targeting polypeptides and cells comprising such nucleic acids. Described are methods for detection, diagnosis, and treatment of cancer using borealin-targeting polypeptides.

### CROSS-REFERENCE TO RELATED APPLICATIONS

This application claims the benefit of priority of U.S. Provisional Patent Application No. 63/252,883 filed Oct. 6, 2021, which is hereby incorporated by reference in its entirety.

### BACKGROUND

### I. Field of the Invention

Aspects of the invention relate to at least the fields of cancer biology and medicine.

### II. Background

The Chromosomal Passenger Complex (CPC) is a protein complex that plays a fundamental role in assuring the maintenance of high fidelity cell division by regulating myriad chromosomal and cytoskeletal events. These processes include spindle and kinetochore assembly, error checking, and maintenance activities by dynamically locating to different subcellular locations. The CPC acts as a central node of cell division activity by recruiting other macromolecular complexes to targeted sites where they act in concert with the CPC to induce particular mitotic functions.

There exists a need for compositions for effective targeting of one or more components of the CPC, as well as methods for use of such compositions for in vitro and in vivo applications.

### SUMMARY

As disclosed herein, borealin, a component of the CPC, is displayed on the surface of cancer cells but not on the surface of normal cells. Accordingly, aspects of the present disclosure are directed to methods and compositions for targeting borealin. Also disclosed are methods for detection, diagnosis, and treatment of cancer using borealin-targeting compositions. Aspects of the disclosure relate to novel antibody and antigen binding fragments. Further aspects relate to polypeptides comprising the antigen binding fragment(s) of the disclosure, and compositions comprising the polypeptides, antibodies, antigen binding fragments, CARs, nucleic acids, or cells of the disclosure.

Aspects of the disclosure also describe a chimeric antigen receptor (CAR) comprising: a) an extracellular binding domain comprising a polypeptide of the disclosure; b) a transmembrane domain; and, c) a cytoplasmic region comprising a costimulatory domain and a primary intracellular signaling domain.

Also described is a method of a making a cell comprising transferring one or more nucleic acid(s) of the disclosure into a cell. In some embodiments, the method further comprises culturing the cell under conditions that allow for expression of a polypeptide from the nucleic acid. In some embodiments, the method further comprising isolating the expressed polypeptide. Further aspects relate to a method for producing a polypeptide comprising transferring a nucleic acid(s) or vector of the disclosure into a cell and isolating polypeptides expressed from the nucleic acid. Aspects relate to a nucleic acid encoding an antibody heavy chain, light chain, or antigen binding fragment, wherein the nucleic acid has at least 70% sequence identity to one of SEQ ID NOS:93-121 or a fragment thereof. In some aspects, the nucleic acid has or has at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOS:93-121. Nucleic acids encoding the polypeptides, antibodies, antigen-binding fragments, scFvs, Fabs, and CARs are also provided.

The cell may be further defined as an immune cell, a NK cell, a human cell, B cell, T cell, Chinese hamster ovary, NS0 murine myeloma cell, PER.C6 cell, or a cell described herein. Also described is a method for treating or preventing cancer in a subject, the method comprising administering to the subject the antibody or antigen binding fragment, polypeptide, composition, host cell, or nucleic acid of the disclosure. Further aspects relate to a method for evaluating a sample from a subject, the method comprising contacting a biological sample from the subject, or extract thereof, with at least one antibody, antigen binding fragment, or polypeptide of the disclosure. Also described is a method for diagnosing cancer in a subject, the method comprising contacting a biological sample from the subject, or extract thereof, with at least one antibody, antigen binding fragment, or polypeptide of the disclosure.

Aspects of the disclosure relate to an antibody or antigen binding fragment comprising a heavy chain variable region and a light chain variable region, wherein the heavy chain variable region comprises a HCDR1, HCDR2, and HCDR3 from a heavy chain variable region of a clone of Table 1 and wherein the light chain variable region comprises a LCDR1, LCDR2, and LCDR3 from the light chain variable region of the same clone of Table 1. Further aspects relate to an antibody or antigen binding fragment comprising a heavy chain variable region and a light chain variable region, wherein the heavy chain variable region comprises a HCDR1, HCDR2, and HCDR3 having or having at least 80% sequence identity or having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with a HCDR1, HCDR2, and HCDR3 from a heavy chain variable region of a clone of Table 1 and wherein the light chain variable region comprises a LCDR1, LCDR2, and LCDR3 having or having at least 80% sequence identity or having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with a LCDR1, LCDR2, and LCDR3 from the light chain variable region of the same clone of Table 1. The HCDR1, HCDR2, HCDR3, LCDR1, LCDR2, and/or LCDR3 may be determined from the variable region sequences by methods known in the art. In some aspects, the CDR is HCDR1, HCDR2, HCDR3, LCDR1, LCDR2, and/or LCDR3 determined by the Chothia method. In some aspects, the CDR is HCDR1, HCDR2, HCDR3, LCDR1, LCDR2, and/or LCDR3 determined by the Kabat method. In some aspects, the CDR is HCDR1, HCDR2, HCDR3, LCDR1, LCDR2, and/or LCDR3 determined by the IMGT method.

Aspects of the disclosure relate to an antibody or antigen binding fragment in which the HCDR1, HCDR2, HCDR2, LCDR1, LCDR2, and LCDR3 each comprise an amino acid sequence that has at least 80% sequence identity to an HCDR1, HCDR2, HCDR2, LCDR1, LCDR2, and LCDR3 of Table 1, wherein the HCDR1, HCDR2, HCDR2, LCDR1, LCDR2, and LCDR3 are from the same clone. In some aspects, the HCDR1, HCDR2, HCDR2, LCDR1, LCDR2, and LCDR3 each comprise an amino acid sequence that has or has at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to an HCDR1, HCDR2, HCDR2, LCDR1, LCDR2, and LCDR3 of Table 1, wherein the HCDR1, HCDR2, HCDR2, LCDR1, LCDR2, and LCDR3 are from the same clone. In some aspects, the HCDR1, HCDR2, HCDR2, LCDR1, LCDR2, and LCDR3 each comprise the amino acid sequence of an HCDR1, HCDR2, HCDR2, LCDR1, LCDR2, and LCDR3 of Table 1, wherein the HCDR1, HCDR2, HCDR2, LCDR1, LCDR2, and LCDR3 are from the same clone.

Aspects of the disclosure relate to an antibody or antigen binding fragment comprising a heavy chain variable region and a light chain variable region, wherein the heavy chain variable region comprises a HCDR1, HCDR2, and HCDR3 having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the HCDR1, HCR2, HCR3 from a heavy chain variable region of a clone of Table 1 and wherein the light chain variable region comprises a LCDR1, LCDR2, and LCDR3 having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the LCDR1, LCDR2, and LCDR3 from the light chain variable region of the same clone of Table 1. In some aspects, the antibody or antigen binding fragment comprises a heavy chain variable region and a light chain variable region, wherein the heavy chain variable region comprises a HCDR1, HCDR2, and HCDR3 having or having at least 80% sequence identity to the HCDR1, HCR2, HCR3 from a heavy chain variable region of a clone of Table 1 and wherein the light chain variable region comprises a LCDR1, LCDR2, and LCDR3 having at least 80% sequence identity to the LCDR1, LCDR2, and LCDR3 from the light chain variable region of the same clone of Table 1. In some aspects, the heavy chain variable region comprises a HCDR1, HCDR2, and HCDR3 having the amino acid sequence of an of a HCDR1, HCDR2, and HCDR3 of a clone of Table 1 and the light chain variable region comprises a LCDR1, LCDR2, and LCDR3 comprising the amino acid sequence of the LCDR1, LCDR2, and LCDR3 from the light chain variable region of the same clone of Table 1.

The polypeptides of the disclosure may comprise at least two antigen binding fragments, wherein each antigen binding fragment is independently selected from an antigen binding fragment of the disclosure. In some aspects, the polypeptide is multivalent. In some aspects, the polypeptide is multispecific. In some aspects, the polypeptide is bispecific. In some aspects, the polypeptide comprises, comprises at least, or comprises at most 1, 2, 3, 4, 5, 6, 7, 8, 9, or 10 antigen binding regions. Each antigen binding region may be independently selected from an antigen binding region of the disclosure. In some aspects, the polypeptide may have repeated units of the same antigen binding region, such as at least, at most, or exactly 1, 2, 3, 4, 5, 6, 7, 8, 9, or 10 repeated units.

In some aspects, the heavy chain variable region comprises an amino acid sequence with at least 80% sequence identity to a heavy chain variable region of a clone of Table 1 and/or the light chain variable region comprises an amino acid sequence with at least 80% sequence identity to the light chain variable region of the same clone of Table 1. In some aspects, the heavy chain variable region comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a heavy chain variable region of a clone of Table 1 and/or the light chain variable region comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the light chain variable region of the same clone of Table 1. In some aspects, the heavy chain variable region comprises the amino acid sequence of a heavy chain variable region of a clone of Table 1 and/or the light chain variable region comprises the amino acid sequence of the same clone of Table 1. In some aspects, the antibody or antigen binding fragment comprises a heavy chain framework region (HFR) 1, HFR2, HFR3, and HFR4 and light chain framework region (LFR) 1, LFR2, LFR3, and LFR4, and wherein the HFR1, HFR2, HFR3, and HFR4 comprises an amino acid sequence with at least 80% sequence identity to an HFR1, HFR2, HFR3, and HFR4, respectively, of a clone of Table 1, and the LFR1, LFR2, LFR3, and LFR4 comprises an amino acid sequence with at least 80% sequence identity to the LFR1, LFR2, LFR3, and LFR4, respectively, of the same clone of Table 1. In some aspects, the antibody or antigen binding fragment comprises a heavy chain framework region (HFR) 1, HFR2, HFR3, and HFR4 and light chain framework region (LFR) 1, LFR2, LFR3, and LFR4, and wherein the HFR1, HFR2, HFR3, and HFR4 comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to an HFR1, HFR2, HFR3, and HFR4, respectively, of a clone of Table 1, and the LFR1, LFR2, LFR3, and LFR4 comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the LFR1, LFR2, LFR3, and LFR4, respectively, of the same clone of Table 1. In some aspects, the HFR1, HFR2, HFR3, and HFR4 comprises the amino acid sequence of an HFR1, HFR2, HFR3, and HFR4, respectively, of a clone of Table 1, and the LFR1, LFR2, LFR3, and LFR4 comprises the amino acid sequence of the LFR1, LFR2, LFR3, and LFR4, respectively, of the same clone of Table 1. In some aspects, the antibody or antigen binding fragment comprises a heavy chain and a light chain and wherein the heavy chain comprises an amino acid sequence with at least 70% sequence identity to a heavy chain of a clone of Table 1 and the light chain comprises an amino acid sequence with at least 70% sequence identity to the light chain of the same clone of Table 1. In some aspects, the antibody or antigen binding fragment comprises a heavy chain and a light chain and wherein the heavy chain comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a heavy chain of a clone of Table 1 and the light chain comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the light chain of the same clone of Table 1. In some aspects, the antibody or antigen binding fragment comprises a heavy chain and a light chain and wherein the heavy chain comprises the amino acid sequence of a clone of Table 1 and the light chain comprises the amino acid sequence of the same clone of Table 1.

In some aspects, the heavy chain variable region comprises a heavy chain framework region that has or has at least 80% sequence identity to a heavy chain framework region of a clone of Table 1 and the light chain variable region comprises a light chain framework region that has or has at least 80% sequence identity to a light chain framework region of the same clone of Table 1. In some aspects, the heavy chain variable region comprises a heavy chain framework region having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a heavy chain framework region of a clone of Table 1 and the light chain variable region comprises a light chain framework region having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a light chain framework region of the same clone of Table 1.

In some aspects, the heavy chain variable region comprises at least 70% sequence identity to the heavy chain variable region of a clone of Table 1 and the light chain variable region comprises at least 70% sequence identity to the light chain variable region of the same clone of Table 1, and wherein the heavy chain and light chain comprise 100% sequence identity to each of the three heavy chain CDRs and three light chain CDRs from the same clone of Table 1. In some aspects, the heavy chain variable region comprises at least 75% sequence identity to the heavy chain variable region of a clone of Table 1 and the light chain variable region comprises at least 75% sequence identity to the light chain variable region of the same clone of Table 1, and wherein the heavy chain and light chain comprise 100% sequence identity to each of the three heavy chain CDRs and three light chain CDRs from the same clone of Table 1. In some aspects, the heavy chain variable region comprises at least 80% sequence identity to the heavy chain variable region of a clone of Table 1 and the light chain variable region comprises at least 80% sequence identity to the light chain variable region of the same clone of Table 1, and wherein the heavy chain and light chain comprise 100% sequence identity to each of the three heavy chain CDRs and three light chain CDRs from the same clone of Table 1. In some aspects, the heavy chain variable region comprises at least 85% sequence identity to the heavy chain variable region of a clone of Table 1 and the light chain variable region comprises at least 85% sequence identity to the light chain variable region of the same clone of Table 1, and wherein the heavy chain and light chain comprise 100% sequence identity to each of the three heavy chain CDRs and three light chain CDRs from the same clone of Table 1. In some aspects, the heavy chain variable region comprises at least 90% sequence identity to the heavy chain variable region of a clone of Table 1 and the light chain variable region comprises at least 90% sequence identity to the light chain variable region of the same clone of Table 1, and wherein the heavy chain and light chain comprise 100% sequence identity to each of the three heavy chain CDRs and three light chain CDRs from the same clone of Table 1. In some aspects, the heavy chain variable region comprises at least 95% sequence identity to the heavy chain variable region of a clone of Table 1 and the light chain variable region comprises at least 95% sequence identity to the light chain variable region of the same clone of Table 1, and wherein the heavy chain and light chain comprise 100% sequence identity to each of the three heavy chain CDRs and three light chain CDRs from the same clone of Table 1.

In some aspects, the Fab or polypeptide comprises the heavy and light chain of a Fab clone of Table 4 or a polypeptide or Fab comprising a heavy and light chain having at least 80% sequence identity to a heavy and light chain of a clone of Table 4. In some aspects, the heavy chain comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a heavy chain of a clone of Table 4 and the light chain comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100/o (or any derivable range therein) sequence identity to a light chain of the same clone of Table 4. In some aspects, the antibody or antigen binding fragment comprises a heavy chain framework region 1 (HFRW1), wherein the HFRW1 comprises an amino acid substitution with a D or T at the carboxy-terminal amino acid of the HFRW1.

The antibody or antigen binding fragment of the disclosure may be human, chimeric, or humanized. In some embodiments, the antibody, or antigen binding fragment binds a borealin protein or antigen with a K_{D} of about 10⁻⁶ M to about 10⁻¹² M. In some embodiments, the antibody, or antigen binding fragment binds a borealin protein or antigen with a K_{D} of about, a K_{D} of at least, or a K_{D} of at most 10, 10⁻¹, 10⁻², 10⁻³, 10⁻⁴, 10⁻⁵, 10⁻⁶, 10⁻⁷, 10⁻¹, 10⁻⁹, 10⁻¹⁰, 10⁻¹¹, 10⁻¹², 10⁻¹³, 10⁻¹⁴, 10⁻¹⁵, 10⁻¹⁶, 10⁻¹⁷, or 10⁻⁸ (or any derivable range therein) M, μM, nM, or pM. In some embodiments, the antibody or antigen binding fragment is further defined as a human antibody or antigen binding fragment, humanized antibody or antigen binding fragment, recombinant antibody or antigen binding fragment, chimeric antibody or antigen binding fragment, an antibody or antigen binding fragment derivative, a veneered antibody or antigen binding fragment, a diabody, a monoclonal antibody or antigen binding fragment, a single domain antibody, or a single chain antibody. In some embodiments, the antigen binding fragment is further defined as a single chain variable fragment (scFv), F(ab′)₂, Fab′, Fab, Fv, or rIgG. In some embodiments, the antibody, antigen binding fragment, or polypeptide is operatively linked to a detectable label. Detectable labels are described herein.

Embodiments of the disclosure also relate to multi-specific and/or multivalent antibodies and polypeptides. Accordingly, embodiments relate to bivalent or bispecific antibodies that comprise two antigen binding fragments, wherein the antigen binding fragment is two of the same antigen binding fragments or two different antigen binding fragments described herein. The disclosure also provides for multi-specific polypeptides. Embodiments relate to polypeptides comprising at least 2, 3, 4, 5, or 6 antigen binding fragments. The antigen binding fragment may be at least 2, 3, r, 5, or 6 scFv, F(ab′)₂, Fab′, Fab, Fv, or rIgG, or combinations thereof.

Polypeptides of the disclosure may comprise antibodies, antigen binding fragments, scFvs, or CARs of the disclosure. In polypeptides of the disclosure, the VH may be amino proximal to the VL. In some aspects, the VL is amino proximal to the VH. The VH and the VL region may be on the same polypeptide. In some aspects, the VH and the VL are separated by a peptide linker. The linker may be a glycine-serine linker. In some aspects, the linker may be a linker described herein. The linker may comprise, comprise at least, or comprise at most 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids, or any derivable range therein. Polypeptides and CARs of the disclosure may comprise or further comprise a second scFv. In some aspects, the second scFv specifically binds to CD3. Polypeptides of the disclosure may comprise or further comprise a CD3 binding region. In some aspects, the CD3 binding region is a scFv. The second scFv may be amino proximal to the first scFv. In some aspects, the first scFv is amino proximal to the second scFv.

The cytoplasmic region in the CARs of the disclosure may comprise or further comprise a co-stimulatory region between the transmembrane domain and the cytoplasmic region. In some aspects, the transmembrane domain comprises a transmembrane domain of CD28. The primary intracellular signaling domain may comprise a CD28 or CD3 zeta signaling domain. In some aspects, the CAR comprises a peptide spacer between the extracellular binding domain and the transmembrane domain. In some aspects, the peptide spacer comprises less than 50 amino acids. The peptide spacer may comprise, comprise at least, or comprise at most 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 amino acids, or any derivable range therein. In some aspects, the peptide spacer comprises more than 50 amino acids. In some aspects, the peptide spacer comprises the hinge region of an IgG molecule. In some aspects, the peptide spacer comprises the hinge and CH₂CH₃ region of an IgG molecule. In some aspects, the CAR is multispecific. In some aspects, the CAR is bispecific. The CAR may comprise an antigen binding domain, wherein the antigen binding domain is an anti-CD3, anti-CD56, anti-CD8 antigen binding domain, and combinations thereof.

The cell may be a human cell, B cell, T cell, Chinese hamster ovary, engineered cell, or immune cell. In some aspects, the cell is a natural killer (NK) cell. The immune cell may be a T cell. In some aspects, the T cell is a CD8⁺ T cell, CD4+ T cell, iNKT, or γδ T cell. In some aspects, the cell is an engineered cell. In some aspects, the engineered cell is an engineered T cell. In some aspects, the cell is a cell type or cell population described herein.

Compositions of the disclosure, such as pharmaceutical compositions may comprise a pharmaceutical excipient, carrier, or molecule described herein, such as the antibodies, antigen binding fragments, polypeptides, scFvs, or CARs of the disclosure. In some aspects, the composition further comprises an adjuvant or an immunostimulator. Such adjuvants or immunostimulators may include, but are not limited to stimulators of pattern recognition receptors, such as Toll-like receptors, RIG-1 and NOD-like receptors (NLR), mineral salts, such as alum, alum combined with monphosphoryl lipid (MPL) A of Enterobacteria, such as Escherichia coli, Salmonella minnesota, Salmonella typhimurium, or Shigella flexneri or specifically with MPL (ASO4), MPL A of above-mentioned bacteria separately, saponins, such as QS-21, Quil-A, ISCOMs, ISCOMATRIX, emulsions such as MF59, Montanide, ISA 51 and ISA 720, AS02 (QS21+squalene+MPL.), liposomes and liposomal formulations such as AS01, synthesized or specifically prepared microparticles and microcarriers such as bacteria-derived outer membrane vesicles (OMV) of N. gonorrhoeae, Chlamydia trachomatis and others, or chitosan particles, depot-forming agents, such as Pluronic block co-polymers, specifically modified or prepared peptides, such as muramyl dipeptide, aminoalkyl glucosaminide 4-phosphates, such as RC529, or proteins, such as bacterial toxoids or toxin fragments. Compositions may comprise more than one antibody and/or antigen binding fragment of the disclosure. Accordingly, compositions of the disclosure may comprise, may comprise at least, or may comprise at most 1, 2, 3, 4, 5, 6, 7, 8, 9, or 10 antibodies and/or antigen binding fragments of the disclosure.

In some embodiments of the disclosure, the method further comprises incubating the antibody, antigen binding fragment, or polypeptide under conditions that allow for the binding of the antibody, antigen binding fragment, or polypeptide to antigens in the biological sample or extract thereof. In some embodiments, the method further comprises detecting the binding of an antigen to the antibody, antigen binding fragment, or polypeptide. In some embodiments, the method further comprises contacting the biological sample with at least one capture antibody, antigen, or polypeptide. The at least one capture antibody, antigen binding fragment, or polypeptide may be an antibody, polypeptide, or antigen binding fragment of the disclosure. In some embodiments, the capture antibody is linked or operatively linked to a solid support. The term “operatively linked” refers to a situation where two components are combined or capable of combining to form a complex. For example, the components may be covalently attached and/or on the same polypeptide, such as in a fusion protein or the components may have a certain degree of binding affinity for each other, such as a binding affinity that occurs through van der Waals forces. In some embodiments, the biological sample comprises a blood sample, urine sample, fecal sample, or nasopharyngeal sample. In embodiments of the disclosure, the at least one antibody, antigen binding fragment, or polypeptide may be operatively linked to a detectable label. In some embodiments, the method further comprises incubating the antibody, antigen binding fragment, or polypeptide under conditions that allow for the binding of the antibody, antigen binding fragment, or polypeptide to antigens in the biological sample or extract thereof. In some embodiments, the method further comprises detecting the binding of an antigen to the antibody, antigen binding fragment, or polypeptide. In some embodiments, the method further comprises contacting the biological sample with at least one capture antibody, antigen, or polypeptide. In some embodiments, the biological sample comprises a blood sample, urine sample, fecal sample, or nasopharyngeal sample.

The cancer may be breast cancer, cervical cancer, prostate cancer, or leukemia. The subject may have one or more symptoms of cancer. In some aspects, the subject does not have any symptoms of cancer. In some aspects, the subject has been diagnosed with cancer. In some aspects, the subject has not been diagnosed with cancer. In some aspects, the subject has been previously treated for cancer. In some aspects, the subject is administered an additional therapy. In some aspects, the additional therapy comprises radiotherapy, chemotherapy, or immunotherapy. In aspects of the disclosure, the subject or patient may be a human subject or a human patient. In some aspects, the subject or patient is a non-human animal. In some aspects, the non-human animal is a bat, monkey, camel, rat, mouse, rabbit, goat, chicken, bird, cat, dog, The subject may further be defined as an at-risk subject. In some aspects, the cells, such as cells of the disclosure, are administered to the subject and wherein the cells are allogenic.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1, 2, and 3, respectively and the LCDR1, LCDR2, and LCDR3 comprises an amino acid sequence of SEQ ID NOS:4, 5, and 6, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:17, 18, and 19, respectively and the LCDR1, LCDR2, and LCDR3 comprises an amino acid sequence of SEQ ID NOS:4, 5, and 20, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:23, 24, and 25, respectively and the LCDR1, LCDR2, and LCDR3 comprises an amino acid sequence of SEQ ID NOS:4, 5, and 26, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:29, 30, and 31, respectively and the LCDR1, LCDR2, and LCDR3 comprises an amino acid sequence of SEQ ID NOS:4, 5, and 32, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:35, 36, and 37, respectively and the LCDR1, LCDR2, and LCDR3 comprises an amino acid sequence of SEQ ID NOS:4, 5, and 38, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:41, 42, and 43, respectively and the LCDR1, LCDR2, and LCDR3 comprises an amino acid sequence of SEQ ID NOS:4, 5, and 38, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:45, 42, and 46, respectively and the LCDR1, LCDR2, and LCDR3 comprises an amino acid sequence of SEQ ID NOS:4, 5, and 38, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:35, 48, and 49, respectively and the LCDR1, LCDR2, and LCDR3 comprises an amino acid sequence of SEQ ID NOS:4, 5, and 50, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:53, 54, and 55, respectively and the LCDR1, LCDR2, and LCDR3 comprises an amino acid sequence of SEQ ID NOS:4, 5, and 38, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:57, 58, and 59, respectively and the LCDR1, LCDR2, and LCDR3 comprises an amino acid sequence of SEQ ID NOS:4, 5, and 60, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:41, 63, and 64, respectively and the LCDR1, LCDR2, and LCDR3 comprises an amino acid sequence of SEQ ID NOS:4, 5, and 65, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:29, 68, and 69, respectively and the LCDR1, LCDR2, and LCDR3 comprises an amino acid sequence of SEQ ID NOS:4, 5, and 70, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:73, 74, and 75, respectively and the LCDR1, LCDR2, and LCDR3 comprises an amino acid sequence of SEQ ID NOS:4, 5, and 76, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:73, 79, and 80, respectively and the LCDR1, LCDR2, and LCDR3 comprises an amino acid sequence of SEQ ID NOS:4, 5, and 81, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:53, 42, and 84, respectively and the LCDR1, LCDR2, and LCDR3 comprises an amino acid sequence of SEQ ID NOS:4, 5, and 85, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:41, 88, and 89, respectively and the LCDR1, LCDR2, and LCDR3 comprises an amino acid sequence of SEQ ID NOS:4, 5, and 90, respectively.

Aspects of the disclosure relate to an antibody, antigen binding fragment, or polypeptide comprising a heavy chain variable region and a light chain variable region of SEQ ID NOS:7 and 8; SEQ ID NOS:21 and 22; SEQ ID NOS:27 and 28; SEQ ID NOS:33 and 34; SEQ ID NOS:39 and 40; SEQ ID NOS:44 and 40; SEQ ID NOS:47 and 40; SEQ ID NOS:51 and 52; SEQ ID NOS:56 and 40; SEQ ID NOS:61 and 62; SEQ ID NOS:66 and 67; SEQ ID NOS:71 and 72; SEQ ID NOS:77 and 78; SEQ ID NOS:82 and 83; SEQ ID NOS:86 and 87; or SEQ ID NOS:91 and 92.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments described herein in the context of the term “comprising” may also be implemented in the context of the term “consisting of” or “consisting essentially of.”

“Individual, “subject,” and “patient” are used interchangeably and can refer to a human or non-human.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Any embodiment discussed with respect to one aspect of the disclosure applies to other aspects of the disclosure as well and vice versa. For example, any step in a method described herein can apply to any other method. Moreover, any method described herein may have an exclusion of any step or combination of steps. Aspects of an embodiment set forth in the Examples are also embodiments that may be implemented in the context of embodiments discussed elsewhere in a different Example or elsewhere in the application, such as in the Summary, Detailed Description, Claims, and Brief Description of the Drawings.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-B shows components of the Chromosomal Passenger Complex (CPC), and the structure of the CPC complex (PDB:2QFA), highlighting the Survivin, Borealin and INCENP domains that were used in phage-display (FIG. 1).

FIG. 2A-B shows binding for Borealin Fabs to the Borealin-N terminal domain (FIG. 2A), and the Borealin C-terminal domain (FIG. 2B), by single-point ELISA.

FIG. 3A-B shows SPR kinetics for a selection of N-terminal (BOR-N-48; FIG. 3A), and C-terminal Borealin binders (BOR-C-56, BOR-C-61, BOR-C-64; FIG. 3B).

FIG. 4A-F shows cell surface staining by Flow for 5 human cancer cell lines (FIG. 4A-E), and human peripheral blood mononuclear cells, as negative control (FIG. 4F).

FIG. 5 shows cell surface staining by Flow for three murine colon tumor cell lines, CT-26 (left) and MC-38 (middle), and 4T1 (right).

FIG. 6A-B shows results demonstrating that the display of cell surface Borealin increases on cell stress. FIG. 6A shows increased Borealin surface exposure induced by protein overexpression (SLC5A8, MYOX, eGFP). FIG. 6B shows induced Borealin surface display induced by 5-fluorouracil (5-FU) treatment at the indicated concentrations.

FIG. 7 shows tumor cell lysis (measured by LDH release; top) and T cell activation (measured by IL2 release; bottom), from treatment of human breast tumor cells (SKBR3) with BiTEs comprising the sequence from BOR-N-48. HER2 is shown as a positive control and MBP is an isotype control.

FIG. 8A-B shows apoptotic cells, monitored by Caspase 3/7 activity (by InCucyte), after 4T1 treatment with a murine BiTE comprising the sequence from BOR-N-48. MBP is an isotype (negative) control. FIG. 8A shows representative images of apoptotic (green) cells captured by the InCucyte. FIG. 8B shows murine BOR-N-48 BiTE dose-response curves monitored by InCucyte.

FIG. 9A-D shows tumor cell lysis (measured by LDH release; FIG. 9A) and T cell activation (measured by IL2 and IFNg release; FIG. 9B-C), from treatment of human breast tumor cells (HCC-1954) with anti-Borealin human CAR-T cells in vitro. HER2 is used as positive control. MBP is a negative control. A “plug-and-play CAR-T” design was used that was developed in the Kossiakoff lab (FIG. 9D).

FIG. 10A-B shows the applicability of the Borealin Fabs for western blot (WB). Recombinant CPC targets Survivin-N(N-term), Borealin-N(N-term), Borealin-C(C-term) and INCENP-N(N-term) domains were run on SDS-PAGE and transferred into PVDF membrane. Membranes were probed with BOR-N-48 or BOR-C-56 (primary Fab). The assay was developed by using a secondary anti-human-F(ab′)2 IgG-HRP reagent

FIG. 11 shows the applicability of Borealin Fabs for Immunofluorescence (IF) microscopy.

FIG. 12 shows the applicability of BOR-N-48 for immunohistochemistry (IHC). Tumor cell pellets were fixed and embedded in paraffin and mounted on a glass slide followed by BOR-N-48 Fab staining.

### DETAILED DESCRIPTION OF THE INVENTION

Borealin is located in the nucleus and cytoplasm and is involved in cell division. The present disclosure is based in part on the surprising discovery that borealin can be displayed on the surface of cancer cells but is not displayed on the surface of normal cells, making it a uniquely specific cancer cell marker. As disclosed herein, a panel of fragments of antibodies (Fabs) to borealin has been generating using phage display mutagenesis. Bi-specific T-cell Engagers (BiTES) and chimeric antigen receptor T-cells (CARTs) that are directed against these proteins are described herein as effective for killing cancer cells. Accordingly, disclosed are compositions comprising various targeting molecules (e.g., antibodies, antibody fragments, antibody-like molecules, antibody-drug conjugates, chimeric antigen receptors, BiTES, etc.) capable of targeting borealin, as well as methods for use of such molecules for detection, diagnosis, and treatment of cancer.

### I. ANTIBODIES

Aspects of the disclosure relate to antibodies, antigen binding fragments thereof, or polypeptides capable of specifically binding to borealin.

The term “antibody” refers to an intact immunoglobulin of any isotype, or a fragment thereof that can compete with the intact antibody for specific binding to the target antigen, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, see, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986). Additionally, antigenic regions of proteins can also be predicted and identified using standard antigenicity and hydropathy plots.

The term “immunogenic sequence” means a molecule that includes an amino acid sequence of at least one epitope such that the molecule is capable of stimulating the production of antibodies in an appropriate host. The term “immunogenic composition” means a composition that comprises at least one immunogenic molecule (e.g., an antigen or carbohydrate).

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al., Front Immunol. 2013; 4: 302; 2013).

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (κ) and lambda (λ). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH3 being closest to the —COOH end. The isotype of an antibody can be IgM, IgD, IgG, IgA, or IgE and is defined by the heavy chains present of which there are five classifications: mu (μ), delta (δ), gamma (γ), alpha (α), or epsilon (ε) chains, respectively. IgG has several subtypes, including, but not limited to, IgG1, IgG2, IgG3, and IgG4. IgM subtypes include IgM1 and IgM2. IgA subtypes include IgA1 and IgA2.

### A. Types of Antibodies

Antibodies can be whole immunoglobulins of any isotype or classification, chimeric antibodies, or hybrid antibodies with specificity to two or more antigens. They may also be fragments (e.g., F(ab′)2, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins.

The term “monomer” means an antibody containing only one Ig unit. Monomers are the basic functional units of antibodies. The term “dimer” means an antibody containing two Ig units attached to one another via constant domains of the antibody heavy chains (the Fc, or fragment crystallizable, region). The complex may be stabilized by a joining (J) chain protein. The term “multimer” means an antibody containing more than two Ig units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities or they may be bi-specific, meaning the two antigen-binding sites have different antigen specificities.

Bispecific antibodies are a class of antibodies that have two paratopes with different binding sites for two or more distinct epitopes. In some embodiments, bispecific antibodies can be biparatopic, wherein a bispecific antibody may specifically recognize a different epitope from the same antigen. In some embodiments, bispecific antibodies can be constructed from a pair of different single domain antibodies termed “nanobodies”. Single domain antibodies are sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86:7875-7882 (2014), each of which are specifically incorporated herein by reference in their entirety.

Bispecific antibodies can be constructed as: a whole IgG, Fab′2, Fab′PEG, a diabody, or alternatively as scFv. Diabodies and scFvs can be constructed without an Fc region, using only variable domains, potentially reducing the effects of anti-idiotypic reaction. Bispecific antibodies may be produced by a variety of methods including, but not limited to, fusion of hybridomas or linking of Fab′ fragments. See, e.g., Songsivilai and Lachmann, Clin. Exp. Immunol. 79:315-321 (1990); Kostelny et al., J. Immunol. 148:1547-1553 (1992), each of which are specifically incorporated by reference in their entirety.

In certain aspects, the antigen-binding domain may be multispecific or heterospecific by multimerizing with VH and VL region pairs that bind a different antigen. For example, the antibody may bind to, or interact with, (a) a cell surface antigen, (b) an Fc receptor on the surface of an effector cell, or (c) at least one other component. Accordingly, aspects may include, but are not limited to, bispecific, trispecific, tetraspecific, and other multispecific antibodies or antigen-binding fragments thereof that are directed to epitopes and to other targets, such as Fc receptors on effector cells.

In some embodiments, multispecific antibodies can be used and directly linked via a short flexible polypeptide chain, using routine methods known in the art. One such example is diabodies that are bivalent, bispecific antibodies in which the VH and VL domains are expressed on a single polypeptide chain, and utilize a linker that is too short to allow for pairing between domains on the same chain, thereby forcing the domains to pair with complementary domains of another chain creating two antigen binding sites. The linker functionality is applicable for embodiments of triabodies, tetrabodies, and higher order antibody multimers. (see, e.g., Hollinger et al., Proc Natl. Acad. Sci. USA 90:6444-6448 (1993); Polijak et al., Structure 2:1121-1123 (1994); Todorovska et al., J. Immunol. Methods 248:47-66 (2001)).

Bispecific diabodies, as opposed to bispecific whole antibodies, may also be advantageous because they can be readily constructed and expressed in E. coli. Diabodies (and other polypeptides such as antibody fragments) of appropriate binding specificities can be readily selected using phage display (WO94/13804) from libraries. If one arm of the diabody is kept constant, for instance, with a specificity directed against a protein, then a library can be made where the other arm is varied and an antibody of appropriate specificity selected. Bispecific whole antibodies may be made by alternative engineering methods as described in Ridgeway et al., (Protein Eng., 9:616-621, 1996) and Krah et al., (N Biotechnol. 39:167-173, 2017), each of which is hereby incorporated by reference in their entirety.

Heteroconjugate antibodies are composed of two covalently linked monoclonal antibodies with different specificities. See, e.g., U.S. Pat. No. 6,010,902, incorporated herein by reference in its entirety.

The part of the Fv fragment of an antibody molecule that binds with high specificity to the epitope of the antigen is referred to herein as the “paratope.” The paratope consists of the amino acid residues that make contact with the epitope of an antigen to facilitate antigen recognition. Each of the two Fv fragments of an antibody is composed of the two variable domains, VH and VL, in dimerized configuration. The primary structure of each of the variable domains includes three hypervariable loops separated by, and flanked by, Framework Regions (FR). The hypervariable loops are the regions of highest primary sequences variability among the antibody molecules from any mammal. The term hypervariable loop is sometimes used interchangeably with the term “Complementarity Determining Region (CDR).” The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the VL domain are identified as L1, L2, and L3, with L1 occurring at the most distal end and L3 occurring closest to the CL domain. The CDRs may also be given the names CDR-L1 (or LCDR1), CDR-L2 (or LCDR2), and CDR-L3 (or LCDR3). The L3 (CDR-L3) is generally the region of highest variability among all antibody molecules produced by a given organism. The CDRs are regions of the polypeptide chain arranged linearly in the primary structure, and separated from each other by Framework Regions. The amino terminal (N-terminal) end of the VL chain is named FR1. The region identified as FR2 occurs between L1 and L2 hypervariable loops. FR3 occurs between L2 and L3 hypervariable loops, and the FR4 region is closest to the CL domain. This structure and nomenclature is repeated for the VH chain, which includes three CDRs identified as CDR-H1 (or HCDR1), CDR-H2 (or HCDR2), and CDR-H3 (or HCDR3). The majority of amino acid residues in the variable domains, or Fv fragments (VH and VL), are part of the framework regions (approximately 85%). The three dimensional, or tertiary, structure of an antibody molecule is such that the framework regions are more internal to the molecule and provide the majority of the structure, with the CDRs on the external surface of the molecule.

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, “AN ANALYSIS OF THE SEQUENCES OF THE VARIABLE REGIONS OF BENCE JONES PROTEINS AND MYELOMA LIGHT CHAINS AND THEIR IMPLICATIONS FOR ANTIBODY COMPLEMENTARITY,” J Exp Med, vol. 132, no. 2, pp. 211-250, August 1970); Chothia (as described in C. Chothia et al., “Conformations of immunoglobulin hypervariable regions,” Nature, vol. 342, no. 6252, pp. 877-883, December 1989); and IMGT (as described in M.-P. Lefranc et al., “IMGT unique numbering for immunoglobulin and T cell receptor variable domains and Ig superfamily V-like domains,” Developmental & Comparative Immunology, vol. 27, no. 1, pp. 55-77, January 2003). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding.

One skilled in the art can use any of several methods to determine the paratope of an antibody. These methods include:

1) Computational predictions of the tertiary structure of the antibody/epitope binding interactions based on the chemical nature of the amino acid sequence of the antibody variable region and composition of the epitope.

2) Hydrogen-deuterium exchange and mass spectroscopy

3) Polypeptide fragmentation and peptide mapping approaches in which one generates multiple overlapping peptide fragments from the full length of the polypeptide and evaluates the binding affinity of these peptides for the epitope.

4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific VH and VL domains as appropriate.

In certain aspects, affinity matured antibodies are enhanced with one or more modifications in one or more CDRs thereof that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Bio/Technology 10:779 (1992) describes affinity maturation by VH and VL domain shuffling, random mutagenesis of CDR and/or framework residues employed in phage display is described by Rajpal et al., PNAS. 24: 8466-8471 (2005) and Thie et al., Methods Mol Biol. 525:309-22 (2009) in conjugation with computation methods as demonstrated in Tiller et al., Front. Immunol. 8:986 (2017).

Chimeric immunoglobulins are the products of fused genes derived from different species; “humanized” chimeras generally have the framework region (FR) from human immunoglobulins and one or more CDRs are from a non-human source.

In certain aspects, portions of the heavy and/or light chain are identical or homologous to corresponding sequences from another particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity. U.S. Pat. No. 4,816,567; and Morrison et al., Proc. Natl. Acad. Sci. USA 81:6851 (1984). For methods relating to chimeric antibodies, see, e.g., U.S. Pat. No. 4,816,567; and Morrison et al., Proc. Natl. Acad. Sci. USA 81:6851-6855 (1985), each of which are specifically incorporated herein by reference in their entirety. CDR grafting is described, for example, in U.S. Pat. Nos. 6,180,370, 5,693,762, 5,693,761, 5,585,089, and 5,530,101, which are all hereby incorporated by reference for all purposes.

In some embodiments, minimizing the antibody polypeptide sequence from the non-human species optimizes chimeric antibody function and reduces immunogenicity. Specific amino acid residues from non-antigen recognizing regions of the non-human antibody are modified to be homologous to corresponding residues in a human antibody or isotype. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. For use in humans, the V region composed of CDR1, CDR2, and partial CDR3 for both the light and heavy chain variance region from a non-human immunoglobulin, are grafted with a human antibody framework region, replacing the naturally occurring antigen receptors of the human antibody with the non-human CDRs. In some instances, corresponding non-human residues replace framework region residues of the human immunoglobulin. Furthermore, humanized antibodies may comprise residues that are not found in the recipient antibody or in the donor antibody to further refine performance. The humanized antibody may also comprise at least a portion of an immunoglobulin constant region (Fc), typically that of a human immunoglobulin. See, e.g., Jones et al., Nature 321:522 (1986); Riechmann et al., Nature 332:323 (1988); Presta, Curr. Op. Struct. Biol. 2:593 (1992); Vaswani and Hamilton, Ann. Allergy, Asthma and Immunol. 1:105 (1998); Harris, Biochem. Soc. Transactions 23; 1035 (1995); Hurle and Gross, Curr. Op. Biotech. 5:428 (1994); Verhoeyen et al., Science 239:1534-36 (1988).

Intrabodies are intracellularly localized immunoglobulins that bind to intracellular antigens as opposed to secreted antibodies, which bind antigens in the extracellular space.

Polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies or “mAb” refer to an antibody obtained from a population of homogeneous antibodies from an exclusive parental cell, e.g., the population is identical except for naturally occurring mutations that may be present in minor amounts. Each monoclonal antibody is directed against a single antigenic determinant.

### B. Functional Antibody Fragments and Antigen-Binding Fragments

### 1. Antigen-Binding Fragments

Certain aspects relate to antibody fragments, such as antibody fragments that bind to borealin. The term functional antibody fragment includes antigen-binding fragments of an antibody that retain the ability to specifically bind to an antigen. These fragments are constituted of various arrangements of the variable region heavy chain (VH) and/or light chain (VL); and in some embodiments, include constant region heavy chain 1 (CH1) and light chain (CL). In some embodiments, they lack the Fc region constituted of heavy chain 2 (CH2) and 3 (CH3) domains. Embodiments of antigen binding fragments and the modifications thereof may include: (i) the Fab fragment type constituted with the VL, VH, CL, and CH1 domains; (ii) the Fd fragment type constituted with the VH and CH1 domains; (iii) the Fv fragment type constituted with the VH and VL domains; (iv) the single domain fragment type, dAb, (Ward, 1989; McCafferty et al., 1990; Holt et al., 2003) constituted with a single VH or VL domain; (v) isolated complementarity determining region (CDR) regions. Such terms are described, for example, in Harlow and Lane, Antibodies: A Laboratory Manual, Cold Spring Harbor Laboratory, NY (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, 2d ed., Wiley-Liss, Inc. New York, N.Y. (1990); Antibodies, 4:259-277 (2015), each of which are incorporated by reference.

Antigen-binding fragments also include fragments of an antibody that retain exactly, at least, or at most 1, 2, or 3 complementarity determining regions (CDRs) from a light chain variable region. Fusions of CDR-containing sequences to an Fc region (or a CH2 or CH3 region thereof) are included within the scope of this definition including, for example, scFv fused, directly or indirectly, to an Fc region are included herein.

The term Fab fragment (also “Fab”) means a monovalent antigen-binding fragment of an antibody containing the VL, VH, CL and CH1 domains. The term Fab′ fragment means a monovalent antigen-binding fragment of a monoclonal antibody that is larger than a Fab fragment. For example, a Fab′ fragment includes the VL, VH, CL and CH1 domains and all or part of the hinge region. The term F(ab′)2 fragment means a bivalent antigen-binding fragment of a monoclonal antibody comprising two Fab′ fragments linked by a disulfide bridge at the hinge region. An F(ab′)2 fragment includes, for example, all or part of the two VH and VL domains, and can further include all or part of the two CL and CH1 domains.

The term Fd fragment means a fragment of the heavy chain of a monoclonal antibody, which includes all or part of the VH, including the CDRs. An Fd fragment can further include CH1 region sequences.

The term Fv fragment means a monovalent antigen-binding fragment of a monoclonal antibody, including all or part of the VL and VH, and absent of the CL and CH1 domains. The VL and VH include, for example, the CDRs. Single-chain antibodies (sFv or scFv) are Fv molecules in which the VL and VH regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv)2 means bivalent or bispecific sFv polypeptide chains that include oligomerization domains at their C-termini, separated from the sFv by a hinge region (Pack et al. 1992). The oligomerization domain comprises self-associating a-helices, e.g., leucine zippers, which can be further stabilized by additional disulfide bonds. (scFv)2 fragments are also known as “miniantibodies” or “minibodies.”

A single domain antibody is an antigen-binding fragment containing only a VH or the VL domain. In some instances, two or more VH regions are covalently joined with a peptide linker to create a bivalent domain antibody. The two VH regions of a bivalent domain antibody may target the same or different antigens.

### 2. Fragment Antigen Binding Region, Fab

Fab polypeptides of the disclosure include the Fab antigen binding fragment of an antibody. Unless specifically stated otherwise, the term “Fab” relates to a polypeptide excluding the Fc portion of the antibody. The Fab may be conjugated to a polypeptide comprising other components, such as further antigen binding domains, costimulatory domains, linkers, peptide spacers, transmembrane domains, endodomains, and accessory proteins. Fab polypeptides can be generated using conventional techniques known in the art and are well-described in the literature.

### 3. Fragment Crystallizable Region, Fc

An Fc region contains two heavy chain fragments comprising the CH2 and CH3 domains of an antibody. The two heavy chain fragments are held together by two or more disulfide bonds and by hydrophobic interactions of the CH3 domains. The term “Fc polypeptide” as used herein includes native and mutein forms of polypeptides derived from the Fc region of an antibody. Truncated forms of such polypeptides containing the hinge region that promotes dimerization are included.

C. Polypeptides with antibody CDRs & Scaffolding Domains that Display the CDRs

Antigen-binding peptide scaffolds, such as complementarity-determining regions (CDRs), are used to generate protein-binding molecules in accordance with the embodiments. Generally, a person skilled in the art can determine the type of protein scaffold on which to graft at least one of the CDRs. It is known that scaffolds, optimally, must meet a number of criteria such as: good phylogenetic conservation; known three-dimensional structure; small size; few or no post-transcriptional modifications; and/or be easy to produce, express, and purify. Skerra, J Mol Recognit, 13:167-87 (2000).

The protein scaffolds can be sourced from, but not limited to: fibronectin type III FN3 domain (known as “monobodies”), fibronectin type III domain 10, lipocalin, anticalin, Z-domain of protein A of Staphylococcus aureus, thioredoxin A or proteins with a repeated motif such as the “ankyrin repeat”, the “armadillo repeat”, the “leucine-rich repeat” and the “tetratricopeptide repeat”. Such proteins are described in US Patent Publication Nos. 2010/0285564, 2006/0058510, 2006/0088908, 2005/0106660, and PCT Publication No. WO2006/056464, each of which are specifically incorporated herein by reference in their entirety. Scaffolds derived from toxins from scorpions, insects, plants, mollusks, etc., and the protein inhibitors of neuronal NO synthase (PIN) may also be used.

### D. Antibody Binding

The term “selective binding agent” refers to a molecule that binds to an antigen. Non-limiting examples include antibodies, antigen-binding fragments, scFv, Fab, Fab′, F(ab′)2, single chain antibodies, peptides, peptide fragments and proteins.

The term “binding” refers to a direct association between two molecules, due to, for example, covalent, electrostatic, hydrophobic, and ionic and/or hydrogen-bond interactions, including interactions such as salt bridges and water bridges. “Immunologically reactive” means that the selective binding agent or antibody of interest will bind with antigens present in a biological sample. The term “immune complex” refers the combination formed when an antibody or selective binding agent binds to an epitope on an antigen.

### 1. Affinity/Avidity

The term “affinity” refers the strength with which an antibody or selective binding agent binds an epitope. In antibody binding reactions, this is expressed as the affinity constant (Ka or ka sometimes referred to as the association constant) for any given antibody or selective binding agent. Affinity is measured as a comparison of the binding strength of the antibody to its antigen relative to the binding strength of the antibody to an unrelated amino acid sequence. Affinity can be expressed as, for example, 20-fold greater binding ability of the antibody to its antigen then to an unrelated amino acid sequence. As used herein, the term “avidity” refers to the resistance of a complex of two or more agents to dissociation after dilution. The terms “immunoreactive” and “preferentially binds” are used interchangeably herein with respect to antibodies and/or selective binding agent.

There are several experimental methods that can be used by one skilled in the art to evaluate the binding affinity of any given antibody or selective binding agent for its antigen. This is generally done by measuring the equilibrium dissociation constant (KD or Kd), using the equation KD=koff/kon=[A][B]/[AB]. The term koff is the rate of dissociation between the antibody and antigen per unit time, and is related to the concentration of antibody and antigen present in solution in the unbound form at equilibrium. The term kon is the rate of antibody and antigen association per unit time, and is related to the concentration of the bound antigen-antibody complex at equilibrium. The units used for measuring the KD are mol/L (molarity, or M), or concentration. The Ka of an antibody is the opposite of the KD, and is determined by the equation Ka=1/KD. Examples of some experimental methods that can be used to determine the KD value are: enzyme-linked immunosorbent assays (ELISA), isothermal titration calorimetry (ITC), fluorescence anisotropy, surface plasmon resonance (SPR), and affinity capillary electrophoresis (ACE). The affinity constant (Ka) of an antibody is the opposite of the KD, and is determined by the equation Ka=1/KD.

Antibodies deemed useful in certain embodiments may have an affinity constant (Ka) of about, at least about, or at most about 10⁶, 10⁷, 10⁸, 10⁹, or 10¹⁰ M or any range derivable therein. Similarly, in some embodiments, antibodies may have a dissociation constant of about, at least about or at most about 10⁻⁶, 10⁻⁷, 10⁻⁸, 10⁻⁹, 10⁻¹⁰ M, or any range derivable therein. These values are reported for antibodies discussed herein and the same assay may be used to evaluate the binding properties of such antibodies. An antibody of the invention is said to “specifically bind” its target antigen when the dissociation constant (KD) is ≤10⁻⁸ M. The antibody specifically binds antigen with “high affinity” when the KD is ≤5×10⁻⁹ M, and with “very high affinity” when the KD is ≤5×10⁻¹⁰ M.

### 2. Epitope Specificity

The epitope of an antigen is the specific region of the antigen for which an antibody has binding affinity. In the case of protein or polypeptide antigens, the epitope is the specific residues (or specified amino acids or protein segment) that the antibody binds with high affinity. An antibody does not necessarily contact every residue within the protein. Nor does every single amino acid substitution or deletion within a protein necessarily affect binding affinity. For purposes of this specification and the accompanying claims, the terms “epitope” and “antigenic determinant” are used interchangeably to refer to the site on an antigen to which B and/or T cells respond or recognize. Polypeptide epitopes can be formed from both contiguous amino acids and noncontiguous amino acids juxtaposed by tertiary folding of a polypeptide. An epitope typically includes at least 3, and typically 5-10 amino acids in a unique spatial conformation.

Epitope specificity of an antibody can be determined in a variety of ways. One approach, for example, involves testing a collection of overlapping peptides of 15 amino acids spanning the full sequence of the protein and differing in increments of a small number of amino acids (e.g., 3 to 30 amino acids). The peptides are immobilized in separate wells of a microtiter dish. Immobilization can be accomplished, for example, by biotinylating one terminus of the peptides. This process may affect the antibody affinity for the epitope, therefore different samples of the same peptide can be biotinylated at the N and C terminus and immobilized in separate wells for the purposes of comparison. This is useful for identifying end-specific antibodies. Optionally, additional peptides can be included terminating at a particular amino acid of interest. This approach is useful for identifying end-specific antibodies to internal fragments. An antibody or antigen-binding fragment is screened for binding to each of the various peptides. The epitope is defined as a segment of amino acids that is common to all peptides to which the antibody shows high affinity binding.

### 3. Modification of Antibody Antigen-Binding Domains

It is understood that the antibodies of the present invention may be modified, such that they are substantially identical to the antibody polypeptide sequences, or fragments thereof, and still bind the epitopes of the present invention. Polypeptide sequences are “substantially identical” when optimally aligned using such programs as Clustal Omega, IGBLAST, GAP or BESTFIT using default gap weights, they share at least 80% sequence identity, at least 90% sequence identity, at least 95% sequence identity, at least 96% sequence identity, at least 97% sequence identity, at least 98% sequence identity, or at least 99% sequence identity or any range therein.

As discussed herein, minor variations in the amino acid sequences of antibodies or antigen-binding regions thereof are contemplated as being encompassed by the present invention, providing that the variations in the amino acid sequence maintain at least 75%, more preferably at least 80%, at least 90%, at least 95%, at least 96%, at least 97%, at least 98% and most preferably at least 99% sequence identity. In particular, conservative amino acid replacements are contemplated.

Conservative replacements are those that take place within a family of amino acids that are related in their side chains. Genetically encoded amino acids are generally divided into families based on the chemical nature of the side chain; e.g., acidic (aspartate, glutamate), basic (lysine, arginine, histidine), nonpolar (alanine, valine, leucine, isoleucine, proline, phenylalanine, methionine, tryptophan), and uncharged polar (glycine, asparagine, glutamine, cysteine, serine, threonine, tyrosine). For example, it is reasonable to expect that an isolated replacement of a leucine moiety with an isoleucine or valine moiety, or a similar replacement of an amino acid with a structurally related amino acid in the same family, will not have a major effect on the binding or properties of the resulting molecule, especially if the replacement does not involve an amino acid within a framework site. Whether an amino acid change results in a functional peptide can readily be determined by assaying the specific activity of the polypeptide derivative. Standard ELISA, Surface Plasmon Resonance (SPR), or other antibody binding assays can be performed by one skilled in the art to make a quantitative comparison of antigen binding affinity between the unmodified antibody and any polypeptide derivatives with conservative substitutions generated through any of several methods available to one skilled in the art.

Fragments or analogs of antibodies or immunoglobulin molecules can be readily prepared by those skilled in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Standard methods to identify protein sequences that fold into a known three-dimensional structure are available to those skilled in the art; Dill and McCallum., Science 338:1042-1046 (2012). Several algorithms for predicting protein structures and the gene sequences that encode these have been developed, and many of these algorithms can be found at the National Center for Biotechnology Information (on the World Wide Web at ncbi.nlm.nih.gov/guide/proteins/) and at the Bioinformatics Resource Portal (on the World Wide Web at expasy.org/proteomics). Thus, the foregoing examples demonstrate that those of skill in the art can recognize sequence motifs and structural conformations that may be used to define structural and functional domains in accordance with the invention.

Framework modifications can be made to antibodies to decrease immunogenicity, for example, by “backmutating” one or more framework residues to a corresponding germline sequence.

It is also contemplated that the antigen-binding domain may be multi-specific or multivalent by multimerizing the antigen-binding domain with VH and VL region pairs that bind either the same antigen (multi-valent) or a different antigen (multi-specific).

### E. Chemical Modification of Antibodies

In some aspects, also contemplated are glycosylation variants of antibodies, wherein the number and/or type of glycosylation site(s) has been altered compared to the amino acid sequences of the parent polypeptide. Glycosylation of the polypeptides can be altered, for example, by modifying one or more sites of glycosylation within the polypeptide sequence to increase the affinity of the polypeptide for antigen (U.S. Pat. Nos. 5,714,350 and 6,350,861). In certain embodiments, antibody protein variants comprise a greater or a lesser number of N-linked glycosylation sites than the native antibody. An N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions that eliminate or alter this sequence will prevent addition of an N-linked carbohydrate chain present in the native polypeptide. For example, the glycosylation can be reduced by the deletion of an Asn or by substituting the Asn with a different amino acid. In other embodiments, one or more new N-linked glycosylation sites are created. Antibodies typically have an N-linked glycosylation site in the Fc region.

Additional antibody variants include cysteine variants, wherein one or more cysteine residues in the parent or native amino acid sequence are deleted from or substituted with another amino acid (e.g., serine). Cysteine variants are useful, inter alia, when antibodies must be refolded into a biologically active conformation. Cysteine variants may have fewer cysteine residues than the native antibody and typically have an even number to minimize interactions resulting from unpaired cysteines.

In some aspects, the polypeptides can be pegylated to increase biological half-life by reacting the polypeptide with polyethylene glycol (PEG) or a reactive ester or aldehyde derivative of PEG, under conditions in which one or more PEG groups become attached to the polypeptide. Polypeptide pegylation may be carried out by an acylation reaction or an alkylation reaction with a reactive PEG molecule (or an analogous reactive water-soluble polymer). Methods for pegylating proteins are known in the art and can be applied to the polypeptides of the invention to obtain PEGylated derivatives of antibodies. See, e.g., EP 0 154 316 and EP 0 401 384. In some aspects, the antibody is conjugated or otherwise linked to transthyretin (TTR) or a TTR variant. The TTR or TTR variant can be chemically modified with, for example, a chemical selected from the group consisting of dextran, poly(n-vinyl pyrrolidone), polyethylene glycols, propropylene glycol homopolymers, polypropylene oxide/ethylene oxide co-polymers, polyoxyethylated polyols, and polyvinyl alcohols. As used herein, the term “polyethylene glycol” is intended to encompass any of the forms of PEG that have been used to derivatize other proteins.

### 1. Conjugation

Derivatives of the antibodies and antigen binding fragments that are described herein are also provided. The derivatized antibody or fragment thereof may comprise any molecule or substance that imparts a desired property to the antibody or fragment. The derivatized antibody can comprise, for example, a detectable (or labeling) moiety (e.g., a radioactive, colorimetric, antigenic, or enzymatic molecule, or a detectable bead), a molecule that binds to another molecule (e.g., biotin or streptavidin), a therapeutic or diagnostic moiety (e.g., a radioactive, cytotoxic, or pharmaceutically active moiety), or a molecule that increases the suitability of the antibody for a particular use (e.g., administration to a subject, such as a human subject, or other in vivo or in vitro uses).

Optionally, an antibody or an immunological portion of an antibody can be chemically conjugated to, or expressed as, a fusion protein with other proteins. In some aspects, polypeptides may be chemically modified by conjugating or fusing the polypeptide to serum protein, such as human serum albumin, to increase half-life of the resulting molecule. See, e.g., EP 0322094 and EP 0 486 525. In some aspects, the polypeptides may be conjugated to a diagnostic agent and used diagnostically, for example, to monitor the development or progression of a disease and determine the efficacy of a given treatment regimen. In some aspects, the polypeptides may also be conjugated to a therapeutic agent to provide a therapy in combination with the therapeutic effect of the polypeptide. Additional suitable conjugated molecules include ribonuclease (RNase), DNase I, an antisense nucleic acid, an inhibitory RNA molecule such as a siRNA molecule, an immunostimulatory nucleic acid, aptamers, ribozymes, triplex forming molecules, and external guide sequences. The functional nucleic acid molecules may act as effectors, inhibitors, modulators, and stimulators of a specific activity possessed by a target molecule, or the functional nucleic acid molecules may possess a de novo activity independent of any other molecules.

In some aspects, disclosed are antibodies and antibody-like molecules that are linked to at least one agent to form an antibody conjugate or payload. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules include toxins, therapeutic enzymes, antibiotics, radiolabeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles, or ligands.

a. Conjugate Types

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to be detected, and/or further quantified if desired. Examples of detectable labels include, but not limited to, radioactive isotopes, fluorescers, semiconductor nanocrystals, chemiluminescers, chromophores, enzymes, enzyme substrates, enzyme cofactors, enzyme inhibitors, dyes, metal ions, metal sols, ligands (e.g., biotin, streptavidin or haptens) and the like. Particular examples of labels are, but not limited to, horseradish peroxidase (HRP), fluorescein, FITC, rhodamine, dansyl, umbelliferone, dimethyl acridinium ester (DMAE), Texas red, luminol, NADPH and α- or β-galactosidase. Antibody conjugates include those intended primarily for use in vitro, where the antibody is linked to a secondary binding ligand and/or to an enzyme to generate a colored product upon contact with a chromogenic substrate. Examples of suitable enzymes include, but are not limited to, urease, alkaline phosphatase, (horseradish) hydrogen peroxidase, or glucose oxidase. Preferred secondary binding ligands are biotin and/or avidin and streptavidin compounds. The uses of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference. Molecules containing azido groups may also be used to form covalent bonds to proteins through reactive nitrene intermediates that are generated by low intensity ultraviolet light (Potter & Haley, 1983).

In some aspects, contemplated are immunoconjugates comprising an antibody or antigen-binding fragment thereof conjugated to a cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate). In this way, the agent of interest can be targeted directly to cells bearing cell surface antigen. The antibody and agent may be associated through non-covalent interactions such as through electrostatic forces, or by covalent bonds. Various linkers, known in the art, can be employed in order to form the immunoconjugate. Additionally, the immunoconjugate can be provided in the form of a fusion protein. In one aspect, an antibody may be conjugated to various therapeutic substances in order to target the cell surface antigen. Examples of conjugated agents include, but are not limited to, metal chelate complexes, drugs, toxins and other effector molecules, such as cytokines, lymphokines, chemokines, immunomodulators, radiosensitizers, asparaginase, carboranes, and radioactive halogens.

In antibody drug conjugates (ADC), an antibody (Ab) is conjugated to one or more drug moieties (D) through a linker (L). The ADC may be prepared by several routes, employing organic chemistry reactions, conditions, and reagents known to those skilled in the art, including: (1) reaction of a nucleophilic group of an antibody with a bivalent linker reagent, to form Ab-L, via a covalent bond, followed by reaction with a drug moiety D; and (2) reaction of a nucleophilic group of a drug moiety with a bivalent linker reagent, to form D-L, via a covalent bond, followed by reaction with the nucleophilic group of an antibody. Antibody drug conjugates may also be produced by modification of the antibody to introduce electrophilic moieties, which can react with nucleophilic substituents on the linker reagent or drug. Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate. In yet another aspect, the antibody may be conjugated to a “receptor” (such as streptavidin) for utilization in tumor or cancer cell pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a radionucleotide).

Examples of an antibody-drug conjugates known to a person skilled in the art are pro-drugs useful for the local delivery of cytotoxic or cytostatic agents, i.e. drugs to kill or inhibit tumor cells in the treatment of cancer (Syrigos and Epenetos, Anticancer Res. 19:605-614 (1999); Niculescu-Duvaz and Springer, Adv. Drg. Del. Rev. 26:151-172 (1997); U.S. Pat. No. 4,975,278). In contrast, systematic administration of these unconjugated drug agents may result in unacceptable levels of toxicity to normal cells as well as the target tumor cells (Baldwin et al., Lancet 1:603-5 (1986); Thorpe, (1985) “Antibody Carriers of Cytotoxic Agents in Cancer Therapy: A Review,” In: Monoclonal Antibodies '84: Biological and Clinical Applications, A. Pincera et al., (eds.) pp. 475-506). Both polyclonal antibodies and monoclonal antibodies have been reported as useful in these strategies (Rowland et al., Cancer Immunol. Immunother. 21:183-87 (1986)).

In certain aspects, ADC include covalent or aggregative conjugates of antibodies, or antigen-binding fragments thereof, with other proteins or polypeptides, such as by expression of recombinant fusion proteins comprising heterologous polypeptides fused to the N-terminus or C-terminus of an antibody polypeptide. For example, the conjugated peptide may be a heterologous signal (or leader) polypeptide, e.g., the yeast alpha-factor leader, or a peptide such as an epitope tag (e.g., V5-His). Antibody-containing fusion proteins may comprise peptides added to facilitate purification or identification of the antibody (e.g., poly-His). An antibody polypeptide also can be linked to the FLAG® (Sigma-Aldrich, St. Louis, Mo.) peptide as described in Hopp et al., Bio/Technology 6:1204 (1988), and U.S. Pat. No. 5,011,912. Oligomers that contain one or more antibody polypeptides may be employed as antagonists. Oligomers may be in the form of covalently linked or non-covalently linked dimers, trimers, or higher oligomers. Oligomers comprising two or more antibody polypeptides are contemplated for use. Other oligomers include heterodimers, homotrimers, heterotrimers, homotetramers, heterotetramers, etc. In certain aspects, oligomers comprise multiple antibody polypeptides joined via covalent or non-covalent interactions between peptide moieties fused to the antibody polypeptides. Such peptides may be peptide linkers (spacers), or peptides that have the property of promoting oligomerization. Leucine zippers and certain polypeptides derived from antibodies are among the peptides that can promote oligomerization of antibody polypeptides attached thereto, as described in more detail below.

b. Conjugation Methodology

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates may also be made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bos(p-diazoniumbenzoyl)-ethylenediamine), diisocyanates (such as toluene 2,6-diisocyanate), and bis-active fluorine compounds (such as 1,5-difluoro-2,4-dinitrobenzene). In some aspects, derivatization of immunoglobulins by selectively introducing sulfhydryl groups in the Fc region of an immunoglobulin, using reaction conditions that do not alter the antibody combining site, are contemplated. Antibody conjugates produced according to this methodology are disclosed to exhibit improved longevity, specificity, and sensitivity (U.S. Pat. No. 5,196,066, incorporated herein by reference). Site-specific attachment of effector or reporter molecules, wherein the reporter or effector molecule is conjugated to a carbohydrate residue in the Fc region has also been disclosed in the literature (O'Shannessy et al., 1987).

### II. ANTIBODY PRODUCTION

### A. Antibody Production

Methods for preparing and characterizing antibodies for use in diagnostic and detection assays, for purification, and for use as therapeutics are well known in the art as disclosed in, for example, U.S. Pat. Nos. 4,011,308; 4,722,890; 4,016,043; 3,876,504; 3,770,380; and 4,372,745 (see, e.g., Antibodies: A Laboratory Manual, Cold Spring Harbor Laboratory, 1988; incorporated herein by reference). These antibodies may be polyclonal or monoclonal antibody preparations, monospecific antisera, human antibodies, hybrid or chimeric antibodies, such as humanized antibodies, altered antibodies, F(ab′)2 fragments, Fab fragments, Fv fragments, single-domain antibodies, dimeric or trimeric antibody fragment constructs, minibodies, or functional fragments thereof which bind to the antigen in question. In certain aspects, polypeptides, peptides, and proteins and immunogenic fragments thereof for use in various embodiments can also be synthesized in solution or on a solid support in accordance with conventional techniques. See, for example, Stewart and Young, (1984); Tarn et al, (1983); Merrifield, (1986); and Barany and Merrifield (1979), each incorporated herein by reference.

Methods of making monoclonal antibodies are also well known in the art (Kohler and Milstein, 1975; Harlow and Lane, 1988, U.S. Pat. No. 4,196,265, herein incorporated by reference in its entirety for all purposes). Typically, this technique involves immunizing a suitable animal with a selected immunogenic composition, e.g., a purified or partially purified protein, polypeptide, peptide or domain. Resulting antibody-producing B-cells from the immunized animal, or all dissociated splenocytes, are then induced to fuse with cells from an immortalized cell line to form hybridomas. Myeloma cell lines suited for use in hybridoma-producing fusion procedures preferably are non-antibody-producing and have high fusion efficiency and enzyme deficiencies that render then incapable of growing in certain selective media that support the growth of only the desired fused cells (hybridomas). Typically, the fusion partner includes a property that allows selection of the resulting hybridomas using specific media. For example, fusion partners can be hypoxanthine/aminopterin/thymidine (HAT)-sensitive. Methods for generating hybrids of antibody-producing spleen or lymph node cells and myeloma cells usually comprise mixing somatic cells with myeloma cells in the presence of an agent or agents (chemical or electrical) that promote the fusion of cell membranes. Next, selection of hybridomas can be performed by culturing the cells by single-clone dilution in microtiter plates, followed by testing the individual clonal supernatants (after two to three weeks) for the desired reactivity. Fusion procedures for making hybridomas, immunization protocols, and techniques for isolation of immunized splenocytes for fusion are known in the art.

Other methods of making antibodies are through molecular display technologies. The include technologies such as phage display and yeast surface display, have been the major driving force of the development [Winter, 1994; Boder, 2000; Viti, 2000; Bradbury, 2011]. These enable display of single-chain Fv (scFv) and Fab fragments on the surface of microorganisms and links the phenotype of a variant to its cognate DNA and thus amino acid sequence [McCafferty, 1990; Kang, 1991; Barbas, 1991]. Using these techniques, it is possible to produce a library of an antibody fragment that contains billions of variants and identify variants with a desired binding function from the library in a HTP manner.

In the standard antibody architecture, six surface loops, three each from the heavy-chain and light-chain variable domains constitute the complementarity determining regions (CDRs). Thus, the antibody and its fragments can be considered as “molecular scaffolds” for presenting CDRs. The sequence diversity of CDRs is the primary source of antibody affinity and specificity. In recombinant antibody libraries, the diversity of CDRs can be either derived from natural immune systems or generated by random mutagenesis, and Fv libraries of both types have been successfully used [Barbas, 1992; Knappik, 2000].

Yeast surface display is another important technical development in antibody engineering. Although the library size achievable with this technique is much smaller than that with phage display, yeast surface display coupled with fluorescence activated cell sorter (FACS) allows for HTP, quantitative characterization of antibody affinity, specificity and expression efficiency [Boder, 2000]. The major strengths of phage display include the large size of a library and rapid completion of the selection and propagation cycle (<2 days). Yeast display provides a highly quantitative readout of binding and expression levels using FACS and the ability to precisely define the selection stringency based on equilibrium and kinetic binding parameters as well as expression levels. The integration of these two display methods can provide for an optimal selection of clones that not only are high affinity binders but are stable and express well.

The end products of recombinant antibody screening are the genes encoding Fv and Fab fragments with desired binding characteristics. Bacterial expression systems for Fv and Fab with good yields have been established [Borrebaeck, 1992]. Thus, the phage display clones can be quickly reformatted into an expression cassette for the production of soluble Fv and Fab fragments.

Library design—At least 15-20 residues of CDR residues contribute to antibody binding to its target. Full combinatorial randomization of this many sites together far exceeds the limits of phage and yeast display library diversity. Since the number of sites cannot be significantly reduced, new library strategies have been developed that reduce the 20 natural amino acid genetic code to a smaller subset, while maintaining high functional diversity (Fellouse, 2007). These libraries using a greatly reduced genetic code can produce high affinity Fab and scFv molecules. Although several reduced genetic code libraries have been constructed and tested, the concept has not been optimized for different target classes and especially for the smaller scaffold camelids V_{H}H and FN3 domains (D2). For screening of these novel libraries, we will combine phage display and yeast surface display for optimal efficiency.

Humanized antibody scaffolds—In molecular display approaches only the variable regions of the antigen binding loops (CDRs) of the Fab domain are generally diversified. The main architecture of the domain is held constant. This main architecture can be based on a fully human antibody scaffold. This circumvents the need to humanize the scaffold, as is required by methods involving animal immunizations.

Monoclonal antibodies may be further purified using filtration, centrifugation, and various chromatographic methods such as HPLC or affinity chromatography. Monoclonal antibodies may be further screened or optimized for properties relating to specificity, avidity, half-life, immunogenicity, binding association, binding disassociation, or overall functional properties relative to being a treatment for infection. Thus, monoclonal antibodies may have alterations in the amino acid sequence of CDRs, including insertions, deletions, or substitutions with a conserved or non-conserved amino acid.

The immunogenicity of a particular immunogen composition can be enhanced by the use of non-specific stimulators of the immune response, known as adjuvants. Adjuvants that may be used in accordance with embodiments include, but are not limited to, IL-1, IL-2, IL-4, IL-7, IL-12, -interferon, GMCSP, BCG, aluminum hydroxide, MDP compounds, such as thur-MDP and nor-MDP, CGP (MTP-PE), lipid A, and monophosphoryl lipid A (MPL). Exemplary adjuvants may include complete Freund's adjuvant (a non-specific stimulator of the immune response containing killed Mycobacterium tuberculosis), incomplete Freund's adjuvants, and/or aluminum hydroxide adjuvant. In addition to adjuvants, it may be desirable to co-administer biologic response modifiers (BRM), such as but not limited to, Cimetidine (CIM; 1200 mg/d) (Smith/Kline, PA); low-dose Cyclophosphamide (CYP; 300 mg/m2) (Johnson/Mead, NJ), cytokines such as 0-interferon, IL-2, or IL-12, or genes encoding proteins involved in immune helper functions, such as B-7. A phage-display system can be used to expand antibody molecule populations in vitro. Saiki, et al., Nature 324:163 (1986); Scharf et al., Science 233:1076 (1986); U.S. Pat. Nos. 4,683,195 and 4,683,202; Yang et al., J Mol Biol. 254:392 (1995); Barbas, III et al., Methods: Comp. Meth Enzymol. (1995) 8:94; Barbas, III et al., Proc Natl Acad Sci USA 88:7978 (1991).

### B. Fully Human Antibody Production

Methods are available for making fully human antibodies. Using fully human antibodies can minimize the immunogenic and allergic responses that may be caused by administering non-human monoclonal antibodies to humans as therapeutic agents. In one embodiment, human antibodies may be produced in a non-human transgenic animal, e.g., a transgenic mouse capable of producing multiple isotypes of human antibodies to protein (e.g., IgG, IgA, and/or IgE) by undergoing V-D-J recombination and isotype switching. Accordingly, this aspect applies to antibodies, antibody fragments, and pharmaceutical compositions thereof, but also non-human transgenic animals, B-cells, host cells, and hybridomas that produce monoclonal antibodies. Applications of human antibodies include, but are not limited to, detect a cell expressing an anticipated protein, either in vivo or in vitro, pharmaceutical preparations containing the antibodies of the present invention, and methods of treating disorders by administering the antibodies.

Fully human antibodies can be produced by immunizing transgenic animals (usually mice) that are capable of producing a repertoire of human antibodies in the absence of endogenous immunoglobulin production. Antigens for this purpose typically have six or more contiguous amino acids, and optionally are conjugated to a carrier, such as a hapten. See, for example, Jakobovits et al., Proc. Natl. Acad. Sci. USA 90:2551-2555 (1993); Jakobovits et al., Nature 362:255-258 (1993); Bruggermann et al., Year in Immunol. 7:33 (1993). In one example, transgenic animals are produced by incapacitating the endogenous mouse immunoglobulin loci encoding the mouse heavy and light immunoglobulin chains therein, and inserting into the mouse genome large fragments of human genome DNA containing loci that encode human heavy and light chain proteins. Partially modified animals, which have less than the full complement of human immunoglobulin loci, are then crossbred to obtain an animal having all of the desired immune system modifications. When administered an immunogen, these transgenic animals produce antibodies that are immunospecific for the immunogen but have human rather than murine amino acid sequences, including the variable regions. For further details of such methods, see, for example, International Patent Application Publication Nos. WO 96/33735 and WO 94/02602, which are hereby incorporated by reference in their entirety. Additional methods relating to transgenic mice for making human antibodies are described in U.S. Pat. Nos. 5,545,807; 6,713,610; 6,673,986; 6,162,963; 6,300,129; 6,255,458; 5,877,397; 5,874,299 and 5,545,806; in International Patent Application Publication Nos. WO 91/10741 and WO 90/04036; and in European Patent Nos. EP 546073B1 and EP 546073A1, all of which are hereby incorporated by reference in their entirety for all purposes.

The transgenic mice described above, referred to herein as “HuMAb” mice, contain a human immunoglobulin gene minilocus that encodes unrearranged human heavy (μ and γ) and κ light chain immunoglobulin sequences, together with targeted mutations that inactivate the endogenous μ and κ chain loci (Lonberg et al., Nature 368:856-859 (1994)). Accordingly, the mice exhibit reduced expression of mouse IgM or κ chains and in response to immunization, the introduced human heavy and light chain transgenes undergo class switching and somatic mutation to generate high affinity human IgG κ monoclonal antibodies (Lonberg et al., supra; Lonberg and Huszar, Intern. Ref Immunol. 13:65-93 (1995); Harding and Lonberg, Ann. N.Y. Acad. Sci. 764:536-546 (1995)). The preparation of HuMAb mice is described in detail in Taylor et al., Nucl. Acids Res. 20:6287-6295 (1992); Chen et al., Int. Immunol. 5:647-656 (1993); Tuaillon et al., J. Immunol. 152:2912-2920 (1994); Lonberg et al., supra; Lonberg, Handbook of Exp. Pharmacol. 113:49-101 (1994); Taylor et al., Int. Immunol. 6:579-591 (1994); Lonberg and Huszar, Intern. Ref Immunol. 13:65-93 (1995); Harding and Lonberg, Ann. N.Y. Acad. Sci. 764:536-546 (1995); Fishwild et al., Nat. Biotechnol. 14:845-851 (1996); the foregoing references are herein incorporated by reference in their entirety for all purposes. See further, U.S. Pat. Nos. 5,545,806; 5,569,825; 5,625,126; 5,633,425; 5,789,650; 5,877,397; 5,661,016; 5,814,318; 5,874,299; 5,770,429; and 5,545,807; as well as International Patent Application Publication Nos. WO 93/1227; WO 92/22646; and WO 92/03918, the disclosures of all of which are hereby incorporated by reference in their entirety for all purposes. Technologies utilized for producing human antibodies in these transgenic mice are disclosed also in WO 98/24893, and Mendez et al., Nat. Genetics 15:146-156 (1997), which are herein incorporated by reference. For example, the HCo7 and HCo12 transgenic mice strains can be used to generate human antibodies.

Using hybridoma technology, antigen-specific humanized monoclonal antibodies with the desired specificity can be produced and selected from the transgenic mice such as those described above. Such antibodies may be cloned and expressed using a suitable vector and host cell, or the antibodies can be harvested from cultured hybridoma cells. Fully human antibodies can also be derived from phage-display libraries (as disclosed in Hoogenboom et al., J. Mol. Biol. 227:381 (1991); and Marks et al., J. Mol. Biol. 222:581 (1991)). One such technique is described in International Patent Application Publication No. WO 99/10494 (herein incorporated by reference), which describes the isolation of high affinity and functional agonistic antibodies for MPL- and msk-receptors using such an approach.

### C. Antibody Fragments Production

Antibody fragments that retain the ability to recognize the antigen of interest will also find use herein. A number of antibody fragments are known in the art that comprise antigen-binding sites capable of exhibiting immunological binding properties of an intact antibody molecule and can be subsequently modified by methods known in the arts. Functional fragments, including only the variable regions of the heavy and light chains, can also be produced using standard techniques such as recombinant production or preferential proteolytic cleavage of immunoglobulin molecules. These fragments are known as Fv. See, e.g., Inbar et al., Proc. Nat. Acad. Sci. USA 69:2659-2662 (1972); Hochman et al., Biochem. 15:2706-2710 (1976); and Ehrlich et al., Biochem. 19:4091-4096 (1980).

Single-chain variable fragments (scFvs) may be prepared by fusing DNA encoding a peptide linker between DNAs encoding the two variable domain polypeptides (VL and VH). scFvs can form antigen-binding monomers, or they can form multimers (e.g., dimers, trimers, or tetramers), depending on the length of a flexible linker between the two variable domains (Kortt et al., Prot. Eng. 10:423 (1997); Kort et al., Biomol. Eng. 18:95-108 (2001)). By combining different VL- and VH-comprising polypeptides, one can form multimeric scFvs that bind to different epitopes (Kriangkum et al., Biomol. Eng. 18:31-40 (2001)). Antigen-binding fragments are typically produced by recombinant DNA methods known to those skilled in the art. Although the two domains of the Fv fragment, VL and VH, are coded for by separate genes, they can be joined using recombinant methods by a synthetic linker that enables them to be made as a single chain polypeptide (known as single chain Fv (sFv or scFv); see e.g., Bird et al., Science 242:423-426 (1988); and Huston et al., Proc. Natl. Acad. Sci. USA 85:5879-5883 (1988). Design criteria include determining the appropriate length to span the distance between the C-terminus of one chain and the N-terminus of the other, wherein the linker is generally formed from small hydrophilic amino acid residues that do not tend to coil or form secondary structures. Suitable linkers generally comprise polypeptide chains of alternating sets of glycine and serine residues, and may include glutamic acid and lysine residues inserted to enhance solubility. Antigen-binding fragments are screened for utility in the same manner as intact antibodies. Such fragments include those obtained by amino-terminal and/or carboxy-terminal deletions, where the remaining amino acid sequence is substantially identical to the corresponding positions in the naturally occurring sequence deduced, for example, from a full-length cDNA sequence.

Antibodies may also be generated using peptide analogs of the epitopic determinants disclosed herein, which may consist of non-peptide compounds having properties analogous to those of the template peptide. These types of non-peptide compound are termed “peptide mimetics” or “peptidomimetics”. Fauchere, J. Adv. Drug Res. 15:29 (1986); Veber and Freidinger TINS p. 392 (1985); and Evans et al., J. Med. Chem. 30:1229 (1987). Liu et al. (2003) also describe “antibody like binding peptidomimetics” (ABiPs), which are peptides that act as pared-down antibodies and have certain advantages of longer serum half-life as well as less cumbersome synthesis methods. These analogs can be peptides, non-peptides or combinations of peptide and non-peptide regions. Fauchere, Adv. Drug Res. 15:29 (1986); Veber and Freidiner, TINS p. 392 (1985); and Evans et al., J. Med. Chem. 30:1229 (1987), which are incorporated herein by reference in their entirety for any purpose. Peptide mimetics that are structurally similar to therapeutically useful peptides may be used to produce a similar therapeutic or prophylactic effect. Such compounds are often developed with the aid of computerized molecular modeling. Generally, peptidomimetics of the invention are proteins that are structurally similar to an antibody displaying a desired biological activity, such as the ability to bind a protein, but have one or more peptide linkages optionally replaced by a linkage selected from: —CH2NH—, —CH2S—, —CH2-CH2-, —CH═CH— (cis and trans), COCH2-, —CH(OH)CH2-, and —CH2SO— by methods well known in the art. Systematic substitution of one or more amino acids of a consensus sequence with a D-amino acid of the same type (e.g., D-lysine in place of L-lysine) may be used in certain embodiments of the invention to generate more stable proteins. In addition, constrained peptides comprising a consensus sequence or a substantially identical consensus sequence variation may be generated by methods known in the art (Rizo and Gierasch, Ann. Rev. Biochem. 61:387 (1992), incorporated herein by reference), for example, by adding internal cysteine residues capable of forming intramolecular disulfide bridges which cyclize the peptide.

Once generated, a phage display library can be used to improve the immunological binding affinity of the Fab molecules using known techniques. See, e.g., Figini et al., J. Mol. Biol. 239:68 (1994). The coding sequences for the heavy and light chain portions of the Fab molecules selected from the phage display library can be isolated or synthesized and cloned into any suitable vector or replicon for expression. Any suitable expression system can be used.

### III. OBTAINING ENCODED ANTIBODIES

In some aspects, there are nucleic acid molecule encoding antibody polypeptides (e.g., heavy or light chain, variable domain only, or full-length). These may be generated by methods known in the art, e.g., isolated from B cells of mice that have been immunized and isolated, phage display, expressed in any suitable recombinant expression system and allowed to assemble to form antibody molecules.

### A. Expression

The nucleic acid molecules may be used to express large quantities of recombinant antibodies or to produce chimeric antibodies, single chain antibodies, immunoadhesins, diabodies, mutated antibodies, and other antibody derivatives. If the nucleic acid molecules are derived from a non-human, non-transgenic animal, the nucleic acid molecules may be used for antibody humanization.

### 1. Vectors

In some aspects, contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, or the antigen-binding portion thereof. In some aspects, expression vectors comprising nucleic acid molecules may encode fusion proteins, modified antibodies, antibody fragments, and probes thereof. In addition to control sequences that govern transcription and translation, vectors and expression vectors may contain nucleic acid sequences that serve other functions as well.

To express the antibodies, or antigen-binding fragments thereof, DNAs encoding partial or full-length light and heavy chains are inserted into expression vectors such that the gene area is operatively linked to transcriptional and translational control sequences. In some aspects, a vector that encodes a functionally complete human CH or CL immunoglobulin sequence with appropriate restriction sites engineered so that any VH or VL sequence can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

### 2. Expression Systems

Numerous expression systems exist that comprise at least a part or all of the expression vectors discussed above. Prokaryote- and/or eukaryote-based systems can be employed for use with an embodiment to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

### 3. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. No. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

### 4. Host Cells

In another aspect, contemplated are the use of host cells into which a recombinant expression vector has been introduced. Antibodies can be expressed in a variety of cell types. An expression construct encoding an antibody can be transfected into cells according to a variety of methods known in the art. Vector DNA can be introduced into prokaryotic or eukaryotic cells via conventional transformation or transfection techniques. Some vectors may employ control sequences that allow it to be replicated and/or expressed in both prokaryotic and eukaryotic cells. In certain aspects, the antibody expression construct can be placed under control of a promoter that is linked to T-cell activation, such as one that is controlled by NFAT-1 or NF-κB, both of which are transcription factors that can be activated upon T-cell activation. Control of antibody expression allows T cells, such as tumor-targeting T cells, to sense their surroundings and perform real-time modulation of cytokine signaling, both in the T cells themselves and in surrounding endogenous immune cells. One of skill in the art would understand the conditions under which to incubate host cells to maintain them and to permit replication of a vector. Also understood and known are techniques and conditions that would allow large-scale production of vectors, as well as production of the nucleic acids encoded by vectors and their cognate polypeptides, proteins, or peptides.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

### B. Isolation

The nucleic acid molecule encoding either or both of the entire heavy and light chains of an antibody or the variable regions thereof may be obtained from any source that produces antibodies. Methods of isolating mRNA encoding an antibody are well known in the art. See e.g., Sambrook et al., supra. The sequences of human heavy and light chain constant region genes are also known in the art. See, e.g., Kabat et al., 1991, supra. Nucleic acid molecules encoding the full-length heavy and/or light chains may then be expressed in a cell into which they have been introduced and the antibody isolated.

### IV. ANTIBODIES, ANTIGEN BINDING FRAGMENTS, POLYPEPTIDES CARS

As used herein, a “protein” or “polypeptide” refers to a molecule comprising at least five amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity. The term polypeptide also includes an antibody fragment described herein as well as antibody domains, such as HCDR1 (Heavy chain complementarity determining region 1), HCDR2 (Heavy chain complementarity determining region 2), HCDR3 (Heavy chain complementarity determining region 3), LCDR1 (Light chain complementarity determining region 1), LCDR2 (Light chain complementarity determining region 2), LCDR3 (Light chain complementarity determining region 3), HFRW1 (Heavy chain framework region 1), HFRW2 (Heavy chain framework region 2), HFRW3 (Heavy chain framework region 3), HFRW4 (Heavy chain framework region 4), LFRW1 (Light chain framework region 1), LFRW2 (Light chain framework region 21), LFRW3 (Light chain framework region 3), LFRW4 (Light chain framework region 4), VH (Heavy chain variable region), VL (Light chain variable region), CH (Heavy chain constant region), or CL (Light chain constant region).

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

### A. Signal Peptide

Polypeptides of the present disclosure may comprise a signal peptide. A “signal peptide” refers to a peptide sequence that directs the transport and localization of the protein within a cell, e.g., to a certain cell organelle (such as the endoplasmic reticulum) and/or the cell surface. In some aspects, a signal peptide directs the nascent protein into the endoplasmic reticulum. This is essential if a receptor is to be glycosylated and anchored in the cell membrane. Generally, the signal peptide natively attached to the amino-terminal most component is used (e.g. in an scFv with orientation light chain-linker-heavy chain, the native signal of the light-chain is used).

In some aspects, the signal peptide is cleaved after passage of the endoplasmic reticulum (ER), i.e., is a cleavable signal peptide. In some aspects, a restriction site is at the carboxy end of the signal peptide to facilitate cleavage.

### B. Antigen Binding Domain

Polypeptides of the present disclosure may comprise one or more antigen binding domains. An “antigen binding domain” describes a region of a polypeptide capable of binding to an antigen under appropriate conditions. In some aspects, an antigen binding domain is a single-chain variable fragment (scFv) based on one or more antibodies (e.g., CD20 antibodies). In some aspects, an antigen binding domain comprise a variable heavy (VH) region and a variable light (VL) region, with the VH and VL regions being on the same polypeptide. In some aspects, the antigen binding domain comprises a linker between the VH and VL regions. A linker may enable the antigen binding domain to form a desired structure for antigen binding.

The variable regions of the antigen-binding domains of the polypeptides of the disclosure can be modified by mutating amino acid residues within the VH and/or VL CDR 1, CDR 2 and/or CDR 3 regions to improve one or more binding properties (e.g., affinity) of the antibody. The term “CDR” refers to a complementarity-determining region that is based on a part of the variable chains in immunoglobulins (antibodies) and T cell receptors, generated by B cells and T cells respectively, where these molecules bind to their specific antigen. Since most sequence variation associated with immunoglobulins and T cell receptors is found in the CDRs, these regions are sometimes referred to as hypervariable regions. Mutations may be introduced by site-directed mutagenesis or PCR-mediated mutagenesis and the effect on antibody binding, or other functional property of interest, can be evaluated in appropriate in vitro or in vivo assays. Preferably conservative modifications are introduced and typically no more than one, two, three, four or five residues within a CDR region are altered. The mutations may be amino acid substitutions, additions or deletions.

Framework modifications can be made to the antibodies to decrease immunogenicity, for example, by “backmutating” one or more framework residues to the corresponding germline sequence.

It is also contemplated that the antigen binding domain may be multi-specific or multivalent by multimerizing the antigen binding domain with VH and VL region pairs that bind either the same antigen (multi-valent) or a different antigen (multi-specific).

The binding affinity of the antigen binding region, such as the variable regions (heavy chain and/or light chain variable region), or of the CDRs may be at least 10-5M, 10-6M, 10-7M, 10⁻⁸M, 10⁻⁹M, 10⁻¹⁰M, 10⁻¹¹M, 10⁻¹²M, or 10⁻¹³M. In some aspects, the KD of the antigen binding region, such as the variable regions (heavy chain and/or light chain variable region), or of the CDRs may be at least 10⁻⁵M, 10⁻⁶M, 10⁻⁷M, 10⁻⁸M, 10⁻⁹M, 10⁻¹⁰M, 10⁻¹¹M, 10⁻¹²M, or 10⁻¹³M (or any derivable range therein).

Binding affinity, KA, or KD can be determined by methods known in the art such as by surface plasmon resonance (SRP)-based biosensors, by kinetic exclusion assay (KinExA), by optical scanner for microarray detection based on polarization-modulated oblique-incidence reflectivity difference (OI-RD), or by ELISA.

In some aspects, the polypeptide comprising the humanized binding region has equal, better, or at least 80, 81, 82, 83, 84, 85, 86, 87, 88, 89, 90, 91, 92, 93, 94, 95, 96, 97, 98, 99, 100, 101, 102, 103, 104, 104, 106, 106, 108, 109, 110, 115, or 120% binding affinity and/or expression level in host cells, compared to a polypeptide comprising a non-humanized binding region, such as a binding region from a mouse.

In some aspects, the framework regions, such as FR1, FR2, FR3, and/or FR4 of a human framework can each or collectively have at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) amino acid substitutions, contiguous amino acid additions, or contiguous amino acid deletions with respect to a mouse framework.

In some aspects, the framework regions, such as FR1, FR2, FR3, and/or FR4 of a mouse framework can each or collectively have at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) amino acid substitutions, contiguous amino acid additions, or contiguous amino acid deletions with respect to a human framework.

The substitution may be at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 of FR1, FR2, FR3, or FR4 of a heavy or light chain variable region.

### C. Peptide Spacer

A peptide spacer, such as an extracellular spacer may link an antigen-binding domain to a transmembrane domain. In some aspects, a peptide spacer is flexible enough to allow the antigen-binding domain to orient in different directions to facilitate antigen binding. In one aspect, the spacer comprises the hinge region from IgG. In some aspects, the spacer comprises or further comprises the CH2CH3 region of immunoglobulin and portions of CD3. In some aspects, the CH2CH3 region may have L235E/N297Q or L235D/N297Q modifications, or at least 75%, at least 80%, at least 85%, at least 90%, at least 95%, at least 98%, or 100% amino acid sequence identity of the CH2CH3 region. In some aspects, the spacer is from IgG4. An extracellular spacer may comprise a hinge region.

As used herein, the term “hinge” refers to a flexible polypeptide connector region (also referred to herein as “hinge region”) providing structural flexibility and spacing to flanking polypeptide regions and can consist of natural or synthetic polypeptides. A “hinge” derived from an immunoglobulin (e.g., IgG1) is generally defined as stretching from Glu216 to Pro230 of human IgG1 (Burton (1985) Molec. Immunol., 22: 161-206). Hinge regions of other IgG isotypes may be aligned with the IgG1 sequence by placing the first and last cysteine residues forming inter-heavy chain disulfide (S—S) bonds in the same positions. The hinge region may be of natural occurrence or non-natural occurrence, including but not limited to an altered hinge region as described in U.S. Pat. No. 5,677,425, incorporated by reference herein. The hinge region can include a complete hinge region derived from an antibody of a different class or subclass from that of the CH1 domain. The term “hinge” can also include regions derived from CD8 and other receptors that provide a similar function in providing flexibility and spacing to flanking regions.

The extracellular spacer can have a length of at least, at most, or exactly 4, 5, 6, 7, 8, 9, 10, 12, 15, 16, 17, 18, 19, 20, 20, 25, 30, 35, 40, 45, 50, 75, 100, 110, 119, 120, 130, 140, 150, 160, 170, 180, 190, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, 290, 300, 325, 350, or 400 amino acids (or any derivable range therein). In some aspects, the extracellular spacer consists of or comprises a hinge region from an immunoglobulin (e.g. IgG). Immunoglobulin hinge region amino acid sequences are known in the art; see, e.g., Tan et al. (1990) Proc. Natl. Acad. Sci. USA 87: 162; and Huck et al. (1986) Nucl. Acids Res.

The length of an extracellular spacer may have effects on the CAR's signaling activity and/or the CAR-T cells' expansion properties in response to antigen-stimulated CAR signaling. In some aspects, a shorter spacer such as less than 50, 45, 40, 30, 35, 30, 25, 20, 15, 14, 13, 12, 11, or 10 amino acids is used. In some aspects, a longer spacer, such as one that is at least 50, 60, 70, 80, 90, 100, 110, 120, 130, 140, 150, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, or 290 amino acids may have the advantage of increased expansion in vivo or in vitro.

As non-limiting examples, an immunoglobulin hinge and/or spacer regions can include, comprise, or consist of one of the following amino acid sequences:

TABLE
Exemplary Hinge Regions

| | SEQ ID |
|---|---|
| SEQUENCE | NO: |
| DKTHT | 155 |
| CPPC | 156 |
| CPEPKSCDTPPPCPR | 157 |
| ELKTPLGDTTHT | 158 |
| KSCDKTHTCP | 159 |
| KCCVDCP | 160 |
| KYGPPCP | 161 |
| EPKSCDKTHTCPPCP | 162 |
| ELKTPLGDTTHTCPRCP | 163 |
| SPNMVPHAHHAQ | 164 |
| ESKYGPPCPPCP | 165 |
| EPKSCDKTYTCPPCP | 166 |
| TTTPAPRPPTPAPTIASQPLSLRPEACRPAAGGAVHTRGLDFACD | 167 |
| APEFEGGPSVFLFPPKPKDTLMISRTPEVTCVVVDVSQEDPEVQFNWY | 168 |
| VDGVEVHNAKTKPREEQFQSTYRVVSVLTVLHQDWLNGKEYKCKV | |
| SNKGLPSSIEKTISKAK | |
| GQPREPQVYTLPPSQEEMTKNQVSLTCLVKGFYPSDIAVEWESNGQP | 169 |
| ENNYKTTPPVLDSDGSFFLYSRLTVDKSRWQEGNVFSCSVMHEALHN | |
| HYTQKSLSLSLGK | |

The extracellular spacer can comprise an amino acid sequence of a human IgG1, IgG2, IgG3, or IgG4, hinge region. The extracellular spacer may also include one or more amino acid substitutions and/or insertions and/or deletions compared to a wild-type (naturally-occurring) hinge region. The extracellular spacer can comprise an amino acid sequence derived from human CD8. The extracellular spacer may comprise or further comprise a CH2 region. The extracellular spacer may comprise or further comprise a CH3 region. When the extracellular spacer comprises multiple parts, there may be anywhere from 0-50 amino acids in between the various parts. For example, there may be at least, at most, or exactly 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 amino acids (or any derivable range therein) between the hinge and the CH2 or CH3 region or between the CH2 and CH3 region when both are present. In some aspects, the extracellular spacer consists essentially of a hinge, CH2, and/or CH3 region, meaning that the hinge, CH2, and/or CH3 region is the only identifiable region present and all other domains or regions are excluded, but further amino acids not part of an identifiable region may be present.

### Transmembrane Domain

Polypeptides of the present disclosure may comprise a transmembrane domain. In some aspects, a transmembrane domain is a hydrophobic alpha helix that spans the membrane. Different transmembrane domains may result in different receptor stability.

In some aspects, the transmembrane domain is interposed between the extracellular spacer and the cytoplasmic region. In some aspects, the transmembrane domain is interposed between the extracellular spacer and one or more costimulatory regions. In some aspects, a linker is between the transmembrane domain and the one or more costimulatory regions.

Any transmembrane domain that provides for insertion of a polypeptide into the cell membrane of a eukaryotic (e.g., mammalian) cell may be suitable for use. In some aspects, the transmembrane domain is derived from CD28, CD8, CD4, CD3-zeta, CD134, or CD7.

Exemplary transmembrane domains useful in any of the aspects of the disclosure include those in the table below:

TABLE
Exemplary transmembrane domain sequences

| | | SEQ ID |
|---|---|---|
| Description | Sequence | NO: |
| CD28-derived | FWVLVVVGGVLACYSLLVTVAFIIFWV | 170 |
| CD8 beta derived | LGLLVAGVLVLLVSLGVAIHLCC | 171 |
| CD4 derived | ALIVLGGVAGLLLFIGLGIFFCVRC | 172 |
| CD3 zeta derived | LCYLLDGILFIYGVILTALFLRV | 173 |
| CD28 derived | WVLVVVGGVLACYSLLVTVAFIIFWV | 174 |
| CD134 (OX40) | VAAILGLGLVLGLLGPLAILLALYLL | 175 |
| derived | | |
| CD7 derived | ALPAALAVISFLLGLGLGVACVLA | 176 |

### D. Cytoplasmic Region

After antigen recognition, receptors of the present disclosure may cluster and a signal transmitted to the cell through the cytoplasmic region. In some aspects, the costimulatory domains described herein are part of the cytoplasmic region. In some aspects, the cytoplasmic region comprises an intracellular signaling domain. An intracellular signaling domain may comprise a primary signaling domain and one or more costimulatory domains.

Cytoplasmic regions and/or costimulatory regions suitable for use in the polypeptides of the disclosure include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation by way of binding of the antigen to the antigen binding domain. In some aspects, the cytoplasmic region includes at least one (e.g., one, two, three, four, five, six, etc.) ITAM motif as described herein. In some aspects, the cytoplasmic region includes DAP10/CD28 type signaling chains.

Cytoplasmic regions suitable for use in the polypeptides of the disclosure include immunoreceptor tyrosine-based activation motif (ITAM)-containing intracellular signaling polypeptides. An ITAM motif is YX1X2(L/I), where X1 and X2 are independently any amino acid. In some cases, the cytoplasmic region comprises 1, 2, 3, 4, or 5 ITAM motifs. In some cases, an ITAM motif is repeated twice in an endodomain, where the first and second instances of the ITAM motif are separated from one another by 6 to 8 amino acids, e.g., (YX1X2(L/I))(X3)n(YX1X2(L/I)), where n is an integer from 6 to 8, and each of the 6-8 X3 can be any amino acid.

A suitable cytoplasmic region may be an ITAM motif-containing portion that is derived from a polypeptide that contains an ITAM motif. For example, a suitable cytoplasmic region can be an ITAM motif-containing domain from any ITAM motif-containing protein. Thus, a suitable endodomain need not contain the entire sequence of the entire protein from which it is derived. Examples of suitable ITAM motif-containing polypeptides include, but are not limited to: DAP12, DAP10, FCER1G (Fc epsilon receptor I gamma chain); CD3D (CD3 delta); CD3E (CD3 epsilon); CD3G (CD3 gamma); CD3-zeta; and CD79A (antigen receptor complex-associated protein alpha chain).

Exemplary cytoplasmic regions are known in the art. The cytoplasmic regions shown below also provide examples of regions that may be incorporated in a CAR of the disclosure:

In some aspects, a suitable cytoplasmic region can comprise an ITAM motif-containing portion of the full length DAP12 amino acid sequence. In some aspects, the cytoplasmic region is derived from FCER1G (also known as FCRG; Fc epsilon receptor I gamma chain; Fc receptor gamma-chain; fc-epsilon RI-gamma; fcRgamma; fceRI gamma; high affinity immunoglobulin epsilon receptor subunit gamma; immunoglobulin E receptor, high affinity, gamma chain; etc.). In some aspects, a suitable cytoplasmic region can comprise an ITAM motif-containing portion of the full length FCER1G amino acid sequence.

In some aspects, the cytoplasmic region is derived from T cell surface glycoprotein CD3 delta chain (also known as CD3D; CD3-DELTA; T3D; CD3 antigen, delta subunit; CD3 delta; CD3δ; CD3d antigen, delta polypeptide (TiT3 complex); OKT3, delta chain; T cell receptor T3 delta chain; T cell surface glycoprotein CD3 delta chain; etc.). In some aspects, a suitable cytoplasmic region can comprise an ITAM motif-containing portion of the full length CD3 delta amino acid sequence. In some aspects, the cytoplasmic region is derived from T cell surface glycoprotein CD3 epsilon chain (also known as CD3e, CD3ε; T cell surface antigen T3/Leu-4 epsilon chain, T cell surface glycoprotein CD3 epsilon chain, AI504783, CD3, CD3-epsilon, T3e, etc.). In some aspects, a suitable cytoplasmic region can comprise an ITAM motif-containing portion of the full length CD3 epsilon amino acid sequence. In some aspects, the cytoplasmic region is derived from T cell surface glycoprotein CD3 gamma chain (also known as CD3G, CD3γ, T cell receptor T3 gamma chain, CD3-GAMMA, T3G, gamma polypeptide (TiT3 complex), etc.). In some aspects, a suitable cytoplasmic region can comprise an ITAM motif-containing portion of the full length CD3 gamma amino acid sequence. In some aspects, the cytoplasmic region is derived from T cell surface glycoprotein CD3 zeta chain (also known as CD3Z, CD3ζ, T cell receptor T3 zeta chain, CD247, CD3-ZETA, CD3H, CD3Q, T3Z, TCRZ, etc.). In some aspects, a suitable cytoplasmic region can comprise an ITAM motif-containing portion of the full length CD3 zeta amino acid sequence.

In some aspects, the cytoplasmic region is derived from CD79A (also known as B3-cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). In some aspects, a suitable cytoplasmic region can comprise an ITAM motif-containing portion of the full length CD79A amino acid sequence.

Specific exemplary cytoplasmic regions are known in the art and further shown in the table below.

TABLE
Cytoplasmic Regions

| SEQUENCE | SEQ ID NO: |
|---|---|
| MGGLEPCSRLLLLPLLLAVSGLRPVQAQAQSDCSCSTVSPGVLAGI | 177 |
| VMGDLVLTVLIALAVYFLGRLVPRGRGAAEAATRKQRITETESPY | |
| QELQGQRSDVYSDLNTQRPYYK | |
| MGGLEPCSRLLLLPLLLAVSGLRPVQAQAQSDCSCSTVSPGVLAGI | 178 |
| VMGDLVLTVLIALAVYFLGRLVPRGRGAAEATRKQRITETESPYQE | |
| LQGQRSDVYSDLNTQRPYYK | |
| MGGLEPCSRLLLLPLLLAVSDCSCSTVSPGVLAGIVMGDLVLTVLI | 179 |
| ALAVYFLGRLVPRGRGAAEAATRKQRITETESPYQELQGQRSDVY | |
| SDLNTQRPYYK | |
| MGGLEPCSRLLLLPLLLAVSDCSCSTVSPGVLAGIVMGDLVLTVLI | 180 |
| ALAVYFLGRLVPRGRGAAEATRKQRITETESPYQELQGQRSDVYS | |
| DLNTQRPYYK | |
| MIPAVVLLLLLLVEQAAALGEPQLCYILDAILFLYGIVLTLLYCRLK | 181 |
| IQVRKAAITSYEKSDGVYTGLSTRNQETYETLKHEKPPQ | |
| DGVYTGLSTRNQETYETLKHE | 182 |
| MEHSTFLSGLVLATLLSQVSPFKIPIEELEDRVFVNCNTSITWVEGT | 183 |
| VGTLLSDITRLDLGKRILDPRGIYRCNGTDIYKDKESTVQVHYRMC | |
| QSCVELDPATVAGIIVTDVIATLLLALGVFCFAGHETGRLSGAADT | |
| QALLRNDQVYQPLRDRDDAQYSHLGGNWARNK | |
| MEHSTFLSGLVLATLLSQVSPFKIPIEELEDRVFVNCNTSITWVEGT | 184 |
| VGTLLSDITRLDLGKRILDPRGIYRCNGTDIYKDKESTVQVHYRTA | |
| DTQALLRNDQVYQPLRDRDDAQYSHLGGNWARNK | |
| DQVYQPLRDRDDAQYSHLGGN | 185 |
| MQSGTHWRVLGLCLLSVGVWGQDGNEEMGGITQTPYKVSISGTT | 186 |
| VILTCPQYPGSEILWQHNDKNIGGDEDDKNIGSDEDHLSLKEFSELE | |
| QSGYYVCYPRGSKPEDANFYLYLRARVCENCMEMDVMSVATIVI | |
| VDICITGGLLLLVYYWSKNRKAKAKPVTRGAGAGGRQRGQNKER | |
| PPPVPNPDYEPIRKGQRDLYSGLNQRRI | |
| NPDYEPIRKGQRDLYSGLNQR | 187 |
| MEQGKGLAVLILAIILLQGTLAQSIKGNHLVKVYDYQEDGSVLLTC | 188 |
| DAEAKNITWFKDGKMIGFLTEDKKKWNLGSNAKDPRGMYQCKGS | |
| QNKSKPLQVYYRMCQNCIELNAATISGFLFAEIVSIFVLAVGVYFIA | |
| GQDGVRQSRASDKQTLLPNDQLYQPLKDREDDQYSHLQGNQLRR | |
| N | |
| DQLYQPLKDREDDQYSHLQGN | 189 |
| MKWKALFTAAILQAQLPITEAQSFGLLDPKLCYLLDGILFIYGVILT | 190 |
| ALFLRVKFSRSADAPAYQQGQNQLYNELNLGRREEYDVLDKRRG | |
| RDPEMGGKPRRKNPQEGLYNELQKDKMAEAYSEIGMKGERRRGK | |
| GHDGLYQGLSTATKDTYDALHMQALPPR | |
| MKWKALFTAAILQAQLPITEAQSFGLLDPKLCYLLDGILFIYGVILT | 191 |
| ALFLRVKFSRSADAPAYQQGQNQLYNELNLGRREEYDVLDKRRG | |
| RDPEMGGKPQRRKNPQEGLYNELQKDKMAEAYSEIGMKGERRRG | |
| KGHDGLYQGLSTATKDTYDALHMQALPPR | |
| RVKFSRSADAPAYQQGQNQLYNELNLGRREEYDVLDKRRGRDPE | 192 |
| MGGKPRRKNPQEGLYNELQKDKMAEAYSEIGMKGERRRGKGHD | |
| GLYQGLSTATKDTYDALHMQALPPR | |
| NQLYNELNLGRREEYDVLDKR | 193 |
| EGLYNELQKDKMAEAYSEIGMK | 194 |
| DGLYQGLSTATKDTYDALHMQ | 195 |
| MPGGPGVLQALPATIFLLFLLSAVYLGPGCQALWMHKVPASLMVS | 196 |
| LGEDAHFQCPHNSSNNANVTWWRVLHGNYTWPPEFLGPGEDPNG | |
| TLIIQNVNKSHGGIYVCRVQEGNESYQQSCGTYLRVRQPPPRPFLD | |
| MGEGTKNRIITAEGIILLFCAVVPGTLLLFRKRWQNEKLGLDAGDE | |
| YEDENLYEGLNLDDCSMYEDISRGLQGTYQDVGSLNIGDVQLEKP | |
| MPGGPGVLQALPATIFLLFLLSAVYLGPGCQALWMHKVPASLMVS | 197 |
| LGEDAHFQCPHNSSNNANVTWWRVLHGNYTWPPEFLGPGEDPNE | |
| PPPRPFLDMGEGTKNRIITAEGIILLFCAVVPGTLLLFRKRWQNEKL | |
| GLDAGDEYEDENLYEGLNLDDCSMYEDISRGLQGTYQDVGSLNIG | |
| DVQLEKP | |
| ENLYEGLNLDDCSMYEDISRG | 198 |
| FWVLVVVGGVLACYSLLVTVAFIIFWVRSKRSRLLHSDYMNMTPR | 199 |
| RPGPTRKHYQPYAPPRDFAAYRS | |

### E. Costimulatory Region

Non-limiting examples of suitable costimulatory regions, such as those included in the cytoplasmic region, include, but are not limited to, polypeptides from 4-1BB (CD137), CD28, ICOS, OX-40, BTLA, CD27, CD30, GITR, and HVEM.

A costimulatory region may have a length of at least, at most, or exactly 20, 25, 30, 35, 40, 50, 60, 70, 80, 90, 100, 150, 200, or 300 amino acids or any range derivable therein. In some aspects, the costimulatory region is derived from an intracellular portion of the transmembrane protein 4-1BB (also known as TNFRSF9; CD137; CDw137; ILA; etc.). In some aspects, the costimulatory region is derived from an intracellular portion of the transmembrane protein CD28 (also known as Tp44). In some aspects, the costimulatory region is derived from an intracellular portion of the transmembrane protein ICOS (also known as AILIM, CD278, and CVID1). In some aspects, the costimulatory region is derived from an intracellular portion of the transmembrane protein OX-40 (also known as TNFRSF4, RP5-902P8.3, ACT35, CD134, OX40, TXGPIL). In some aspects, the costimulatory region is derived from an intracellular portion of the transmembrane protein BTLA (also known as BTLA1 and CD272). In some aspects, the costimulatory region is derived from an intracellular portion of the transmembrane protein CD27 (also known as S 152, T14, TNFRSF7, and Tp55). In some aspects, the costimulatory region is derived from an intracellular portion of the transmembrane protein CD30 (also known as TNFRSF8, D1S166E, and Ki-1). In some aspects, the costimulatory region is derived from an intracellular portion of the transmembrane protein GITR (also known as TNFRSF18, RP5-902P8.2, AITR, CD357, and GITR-D). In some aspects, the costimulatory region derived from an intracellular portion of the transmembrane protein HVEM (also known as TNFRSF14, RP3-395M20.6, ATAR, CD270, HVEA, HVEM, LIGHTR, and TR2).

Specific exemplary co-stimulatory domains are represented by the amino acid sequences below:

TABLE
Co-stimulatory domains

| SEQUENCE | SEQ ID NO: |
|---|---|
| KRGRKKLLYIFKQPFMRPVQTTQEEDGCSCRFPEEEEGGCEL | |
| FWVRSKRSRLLHSDYMNMTPRRPGPTRKHYQPYAPPRDFAAYRS | |
| TKKKYSSSVHDPNGEYMFMRAVNTAKKSRLTDVTL | |
| RRDQRLPPDAHKPPGGGSFRTPIQEEQADAHSTLAKI | |
| CCLRRHQGKQNELSDTAGREINLVDAHLKSEQTEASTRQNSQVLL | |
| SETGIYDNDPDLCFRMQEGSEVYSNPCLEENKPGIVYASLNHSVIG | |
| PNSRLARNVKEAPTEYASICVRS | |
| HQRRKYRSNKGESPVEPAEPCRYSCPREEEGSTIPIQEDYRKPEPA | |
| CSP | |
| RRACRKRIRQKLHLCYPVQTSQPKLELVDSRPRRSSTQLRSGASVT | |
| EPVAEERGLMSQPLMETCHSVGAAYLESLPLQDASPAGGPSSPRD | |
| LPEPRVSTEHTNNKIEKIYIMKADTVIVGTVKAELPEGRGLAGPAE | |
| PELEEELEADHTPHYPEQETEPPLGSCSDVMLSVEEEGKEDPLPTA | |
| ASGK | |
| HIWQLRSQCMWPRETQLLLEVPPSTEDARSCQFPEEERGERSAEE | |
| KGRLGDLWV | |
| CVKRRKPRGDVVKVIVSVQRKRQEAEGEATVIEALQAPPDVTTV | |
| AVEETIPSFTGRSPNH | |
| RSKRSRGGHSDYMNMTPRRPGPTRKHYQPYAPPRDFAAYRS | |

### F. Detection Peptides

In some aspects, the polypeptides described herein may further comprise a detection peptide. Suitable detection peptides include hemagglutinin (HA; e.g., YPYDVPDYA (SEQ ID NO:200); FLAG (e.g., DYKDDDDK (SEQ ID NO:201); c-myc (e.g., EQKLISEEDL; SEQ ID NO:202), and the like. Other suitable detection peptides are known in the art.

### G. Peptide Linkers

In some aspects, the polypeptides of the disclosure include peptide linkers (sometimes referred to as a linker). A peptide linker may be used to separate any of the peptide domain/regions described herein. As an example, a linker may be between the signal peptide and the antigen binding domain, between the VH and VL of the antigen binding domain, between the antigen binding domain and the peptide spacer, between the peptide spacer and the transmembrane domain, flanking the costimulatory region or on the N- or C-region of the costimulatory region, and/or between the transmembrane domain and the endodomain. The peptide linker may have any of a variety of amino acid sequences. Domains and regions can be joined by a peptide linker that is generally of a flexible nature, although other chemical linkages are not excluded. A linker can be a peptide of between about 6 and about 40 amino acids in length, or between about 6 and about 25 amino acids in length. These linkers can be produced by using synthetic, linker-encoding oligonucleotides to couple the proteins.

Peptide linkers with a degree of flexibility can be used. The peptide linkers may have virtually any amino acid sequence, bearing in mind that suitable peptide linkers will have a sequence that results in a generally flexible peptide. The use of small amino acids, such as glycine and alanine, are of use in creating a flexible peptide. The creation of such sequences is routine to those of skill in the art.

Suitable linkers can be readily selected and can be of any suitable length, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Example flexible linkers include glycine polymers (G)n, glycine-serine polymers (including, for example, (GS)n, (GSGGS—SEQ ID NO:203)n, (G4S)n and (GGGS—SEQ ID NO:204)n, where n is an integer of at least one. In some aspects, n is at least, at most, or exactly 1, 2, 3, 4, 5, 6, 7, 8, 9, or 10 (or any derivable range therein). Glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains. Exemplary spacers can comprise amino acid sequences including, but not limited to, GGSG (SEQ ID NO:205), GGSGG (SEQ ID NO:206), GSGSG (SEQ ID NO:207), GSGGG (SEQ ID NO:208), GGGSG (SEQ ID NO:209), GSSSG (SEQ ID NO:210), and the like. In further aspects, the linker comprises (EAAAK)n, wherein n is an integer of at least one (SEQ ID NO:211). In some aspects, n is at least, at most, or exactly 1, 2, 3, 4, 5, 6, 7, 8, 9, or 10 (or any derivable range therein).

### H. Additional Modifications and Polypeptide Enhancements

As used herein, a “protein” or “polypeptide” refers to a molecule comprising at least five amino acid residues. As used herein, the term “wild-type” refers to the endogenous version of a molecule that occurs naturally in an organism. In some aspects, wild-type versions of a protein or polypeptide are employed, however, in many aspects of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some aspects, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity. The term polypeptide also includes and antibody fragment described herein as well as antibody domains, such as HCDR1, HCDR2, HCDR3, LCDR1, LCDR2, LCDR3, HFRW1, HFRW2, HFRW3, HFRW4, LFRW1, LFRW2, LFRW3, LFRW4, VH, VL, CH, or CL.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular aspects, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain aspects the size of an antibody, antigen binding fragment, protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The antibody, antigen binding fragment, polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID NO:1-211.

In some aspects, the antibody, antigen binding fragment, protein, or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) of SEQ ID NOS:1-211.

In some aspects, the antibody, antigen binding fragment, or polypeptide may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) contiguous amino acids of SEQ ID NOs:1-211.

In some aspects, the antibody, antigen binding fragment, protein, or polypeptide may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids of SEQ ID NOS:1-211 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of SEQ ID NOS:1-211.

In some aspects there is a nucleic acid molecule, antibody, antigen binding fragment, protein, or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 of any of SEQ ID NOS:1-211 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleotides of any of SEQ ID NOS:1-211.

In some aspects, the amino acid at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 of the heavy chain, light chain, VH, VL, HCDR1, HCDR2, HCDR3, LCDR1, LCDR2, LCDR3, HFRW1, HFRW2, HFRW3, HFRW4, LFRW1, LFRW2, LFRW3, or LFRW4 of SEQ ID NOS:1-211 is substituted with an alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

In some aspects, a polypeptide (e.g., antibody, antibody fragment, Fab, etc.) of the disclosure comprises a CDR that is at least 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical (or any range derivable therein) in sequence to one of SEQ ID NOS1-211. The CDR may be one that has been determined by Kabat, IMGT, or Chothia. In further aspects, a polypeptide may have CDRs that have 1, 2, and/or 3 amino acid changes (e.g., addition of 1 or 2 amino acids, deletions of 1 or 2 amino acids, substitution) with respect to these 1, 2, or 3 CDRs. In some aspects, a polypeptide comprises additionally or alternatively, an amino acid sequence that is at least 60, 65, 70, 75, 80, 85, 90, 95, 96, 97, 98, 99, or 100% identical or homologous to the amino acid sequence of the variable region that is not a CDR sequence, i.e., the variable region framework.

From amino to carboxy terminus the CDRs are CDR1, CDR2, and CDR3. In some aspects, a polypeptide may have CDRs that have 1, 2, and/or 3 amino acid changes (e.g., addition of 1 or 2 amino acids, deletions of 1 or 2 amino acids, substitution) with respect to CDR1, CDR2, or CDR3. In some aspects, the CDRs may further comprise 1, 2, 3, 4, 5, or 6 additional amino acids at the amino or carboxy terminus of the CDR, The additional amino acids may be from the heavy and/or light chain framework regions that are shown as immediately adjacent to the CDRs. Accordingly, aspects relate to polypeptides comprising an HCDR1 (i.e., CDR-H1), HCDR2 (i.e., CDR-H2), HCDR3 (i.e., CDR-H3), LCDR1 (i.e., CDR-L1), LCDR2 (i.e., CDR-L2), and/or LCDR3 (i.e., CDR-L3) with at least or at most or exactly 1, 2, 3, 4, 5, 6 or 7 amino acids at the amino end of the CDR or at the carboxy end of the CDR, wherein the additional amino acids are the 1, 2, 3, 4, 5, 6, or 7 amino acids that are shown as immediately adjacent to the CDRs. Other aspects relate to antibodies comprising one or more CDRs, wherein the CDR is a fragment of one of the CDRs of Table 1 and wherein the fragment lacks 1, 2, 3, 4, or 5 amino acids from the amino or carboxy end of the CDR. In some aspects, the CDR may lack one, 2, 3, 4, 5, 6, or 7 amino acids from the carboxy end and may further comprise 1, 2, 3, 4, 5, 6, 7, or 8 amino acids from the framework region of the amino end of the CDR. In some aspects, the CDR may lack one, 2, 3, 4, 5, 6, or 7 amino acids from the amino end and may further comprise 1, 2, 3, 4, 5, 6, 7, or 8 amino acids from the framework region of the carboxy end of the CDR. In further aspects, an antibody may be alternatively or additionally humanized in regions outside the CDR(s) and/or variable region(s). In some aspects, a polypeptide comprises additionally or alternatively, an amino acid sequence that is at least 60, 65, 70, 75, 80, 85, 90, 95, 96, 97, 98, 99, or 100% identical or homologous to the amino acid sequence of the variable region that is not a CDR sequence, i.e., the variable region framework.

In other aspects, a polypeptide or protein comprises 1, 2, 3, 4, 5, or 6 CDRs from either or both of the light and heavy variable regions of the antigen binding clones of Table 1, and 1, 2, 3, 4, 5, or 6 CDRs may have 1, 2, and/or 3 amino acid changes with respect to these CDRs. In some aspects, parts or all of the antibody sequence outside the variable region have been humanized. A protein may comprise one or more polypeptides. In some aspects, a protein may contain one or two polypeptides similar to a heavy chain polypeptide and/or 1 or 2 polypeptides similar to a light chain polypeptide.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

### I. Borealin

Aspects of the present disclosure are related to compositions and methods for identifying, detecting, and targeting borealin. Borealin (also known as Dasra or Dasra-B) is a protein encoded by the gene CDCA8. Borealin functions in cell division and is a component of the Chromosomal Passenger Complex (CPC). An example mRNA sequence encoding for human borealin is available as NCBI RefSeq NM_001256875. An example protein sequence for human borealin is available as NCBI RefSeq NP_001243804.

### V. SEQUENCES

Polypeptide, antibody, and antigen binding fragment embodiments are shown below in the following tables.

TABLE 1
Antibody and antigen binding embodiments

| | | | SEQ ID |
|---|---|---|---|
| Clone | Region | Sequence | NO: |
| BOR-N- | HCDR1 | IYYSSI | 1 |
| 54 | HCDR2 | SIYPYYGSTS | 2 |
| | HCDR3 | KWYYGDVGM | 3 |
| | LCDR1 | SVSSA | 4 |
| | LCDR2 | SASSLYS | 5 |
| | LCDR3 | SEYRLI | 6 |
| | VH | EISEVQLVESGGGLVQPGGSLRLSCAASGFNIYYSSIHWVR | 7 |
| | | QAPGKGLEWVASIYPYYGSTSYADSVKGRFTISADTSKNT | |
| | | AYLQMNSLRAEDTAVYYCARKWYYGDVGMDYWGQGT | |
| | | LVTVSS | |
| | VL | SDIQMTQSPSSLSASVGDRVTITCRASQSVSSAVAWYQQK | 8 |
| | | PGKAPKLLIYSASSLYSGVPSRFSGSRSGTDFTLTISSLQPE | |
| | | DFATYYCQQSEYRLITFGQGTKVEIKRT | |
| | HFRW1 | EISEVQLVESGGGLVQPGGSLRLSCAASGFN | 9 |
| | HFRW2 | HWVRQAPGKGLEWVA | 10 |
| | HFRW3 | YADSVKGRFTISADTSKNTAYLQMNSLRAEDTAVYYCAR | 11 |
| | HFRW4 | DYWGQGTLVTVSS | 12 |
| | LFRW1 | SDIQMTQSPSSLSASVGDRVTITCRASQ | 13 |
| | LFRW2 | VAWYQQKPGKAPKLLIY | 14 |
| | LFRW3 | GVPSRFSGSRSGTDFTLTISSLQPEDFATYYCQQ | 15 |
| | LFRW4 | TFGQGTKVEIKRT | 16 |
| BOR-C- | HCDR1 | LSSSYI | 17 |
| 59 | HCDR2 | YIYSSSGSTY | 18 |
| | HCDR3 | WPTYYYWLVGWMYDVGAF | 19 |
| | LCDR1 | SVSSA | 4 |
| | LCDR2 | SASSLYS | 5 |
| | LCDR3 | SSSEYPV | 20 |
| | VH | EISEVQLVESGGGLVQPGGSLRLSCAASGFNLSSSYIHWVR | 21 |
| | | QAPGKGLEWVAYIYSSSGSTYYADSVKGRFTISADTSKNT | |
| | | AYLQMNSLRAEDTAVYYCARWPTYYYWLVGWMYDVG | |
| | | AFDYWGQGTLVTVSS | |
| | VL | SDIQMTQSPSSLSASVGDRVTITCRASQSVSSAVAWYQQK | 22 |
| | | PGKAPKLLIYSASSLYSGVPSRFSGSRSGTDFTLTISSLQPE | |
| | | DFATYYCQQSSSEYPVTFGQGTKVEIKRT | |
| | HFRW1 | EISEVQLVESGGGLVQPGGSLRLSCAASGFN | 9 |
| | HFRW2 | HWVRQAPGKGLEWVA | 10 |
| | HFRW3 | YADSVKGRFTISADTSKNTAYLQMNSLRAEDTAVYYCAR | 11 |
| | HFRW4 | DYWGQGTLVTVSS | 12 |
| | LFRW1 | SDIQMTQSPSSLSASVGDRVTITCRASQ | 13 |
| | LFRW2 | VAWYQQKPGKAPKLLIY | 14 |
| | LFRW3 | GVPSRFSGSRSGTDFTLTISSLQPEDFATYYCQQ | 15 |
| | LFRW4 | TFGQGTKVEIKRT | 16 |
| BOR-C- | HCDR1 | FSYSYI | 23 |
| 60 | HCDR2 | YISSSSGYTY | 24 |
| | HCDR3 | SGLYSHGVWQNWYGYMGF | 25 |
| | LCDR1 | SVSSA | 4 |
| | LCDR2 | SASSLYS | 5 |
| | LCDR3 | SLQWPI | 26 |
| | VH | EISEVQLVESGGGLVQPGGSLRLSCAASGFNFSYSYIHWV | 27 |
| | | RQAPGKGLEWVAYISSSSGYTYYADSVKGRFTISADTSKN | |
| | | TAYLQMNSLRAEDTAVYYCARSGLYSHGVWQNWYGYM | |
| | | GFDYWGQGTLVTVSS | |
| | VL | SDIQMTQSPSSLSASVGDRVTITCRASQSVSSAVAWYQQK | 28 |
| | | PGKAPKLLIYSASSLYSGVPSRFSGSRSGTDFTLTISSLQPE | |
| | | DFATYYCQQSLQWPITFGQGTKVEIKRT | |
| | HFRW1 | EISEVQLVESGGGLVQPGGSLRLSCAASGEN | 9 |
| | HFRW2 | HWVRQAPGKGLEWVA | 10 |
| | HFRW3 | YADSVKGRFTISADTSKNTAYLQMNSLRAEDTAVYYCAR | 11 |
| | HFRW4 | DYWGQGTLVTVSS | 12 |
| | LFRW1 | SDIQMTQSPSSLSASVGDRVTITCRASQ | 13 |
| | LFRW2 | VAWYQQKPGKAPKLLIY | 14 |
| | LFRW3 | GVPSRFSGSRSGTDFTLTISSLQPEDFATYYCQQ | 15 |
| | LFRW4 | TFGQGTKVEIKRT | 16 |
| BOR-N- | HCDR1 | VYSYSI | 29 |
| 51 | HCDR2 | SIYSSYSYTS | 30 |
| | HCDR3 | KYHFKPTYWGAM | 31 |
| | LCDR1 | SVSSA | 4 |
| | LCDR2 | SASSLYS | 5 |
| | LCDR3 | SSLYYPL | 32 |
| | VH | EISEVQLVESGGGLVQPGGSLRLSCAASGFNVYSYSIHWV | 33 |
| | | RQAPGKGLEWVASIYSSYSYTSYADSVKGRFTISADTSKN | |
| | | TAYLQMNSLRAEDTAVYYCARKYHFKPTYWGAMDYWG | |
| | | QGTLVTVSS | |
| | VL | SDIQMTQSPSSLSASVGDRVTITCRASQSVSSAVAWYQQK | 34 |
| | | PGKAPKLLIYSASSLYSGVPSRFSGSRSGTDFTLTISSLQPE | |
| | | DFATYYCQQSSLYYPLTFGQGTKVEIKRT | |
| | HFRW1 | EISEVQLVESGGGLVQPGGSLRLSCAASGFN | 9 |
| | HFRW2 | HWVRQAPGKGLEWVA | 10 |
| | HFRW3 | YADSVKGRFTISADTSKNTAYLQMNSLRAEDTAVYYCAR | 11 |
| | HFRW4 | DYWGQGTLVTVSS | 12 |
| | LFRW1 | SDIQMTQSPSSLSASVGDRVTITCRASQ | 13 |
| | LFRW2 | VAWYQQKPGKAPKLLIY | 14 |
| | LFRW3 | GVPSRFSGSRSGTDFTLTISSLQPEDFATYYCQQ | 15 |
| | LFRW4 | TFGQGTKVEIKRT | 16 |
| BOR-N- | HCDR1 | VYYSSI | 35 |
| 55 | HCDR2 | SISPYYGSTS | 36 |
| | HCDR3 | RWYYTKVFMWGM | 37 |
| | LCDR1 | SVSSA | 4 |
| | LCDR2 | SASSLYS | 5 |
| | LCDR3 | SSSSLI | 38 |
| | VH | EISEVQLVESGGGLVQPGGSLRLSCAASGFNVYYSSIHWV | 39 |
| | | RQAPGKGLEWVASISPYYGSTSYADSVKGRFTISADTSKN | |
| | | TAYLQMNSLRAEDTAVYYCARRWYYTKVFMWGMDYW | |
| | | GQGTLVTVSS | |
| | VL | SDIQMTQSPSSLSASVGDRVTITCRASQSVSSAVAWYQQK | 40 |
| | | PGKAPKLLIYSASSLYSGVPSRFSGSRSGTDFTLTISSLQPE | |
| | | DFATYYCQQSSSSLITFGQGTKVEIKRT | |
| | HFRW1 | EISEVQLVESGGGLVQPGGSLRLSCAASGFN | 9 |
| | HFRW2 | HWVRQAPGKGLEWVA | 10 |
| | HFRW3 | YADSVKGRFTISADTSKNTAYLQMNSLRAEDTAVYYCAR | 11 |
| | HFRW4 | DYWGQGTLVTVSS | 12 |
| | LFRW1 | SDIQMTQSPSSLSASVGDRVTITCRASQ | 13 |
| | LFRW2 | VAWYQQKPGKAPKLLIY | 14 |
| | LFRW3 | GVPSRFSGSRSGTDFTLTISSLQPEDFATYYCQQ | 15 |
| | LFRW4 | TFGQGTKVEIKRT | 16 |
| BOR-C- | HCDR1 | VSSYSI | 41 |
| 62 | HCDR2 | SISSSSGSTS | 42 |
| | HCDR3 | ESNQYYYYYNVLWWYQAF | 43 |
| | LCDR1 | SVSSA | 4 |
| | LCDR2 | SASSLYS | 5 |
| | LCDR3 | SSSSLI | 38 |
| | VH | EISEVQLVESGGGLVQPGGSLRLSCAASGFNVSSYSIHWV | 44 |
| | | RQAPGKGLEWVASISSSSGSTSYADSVKGRFTISADTSKNT | |
| | | AYLQMNSLRAEDTAVYYCARESNQYYYYYNVLWWYQA | |
| | | FDYWGQGTLVTVSS | |
| | VL | SDIQMTQSPSSLSASVGDRVTITCRASQSVSSAVAWYQQK | 40 |
| | | PGKAPKLLIYSASSLYSGVPSRFSGSRSGTDFTLTISSLQPE | |
| | | DFATYYCQQSSSSLITFGQGTKVEIKRT | |
| | HFRW1 | EISEVQLVESGGGLVQPGGSLRLSCAASGFN | 9 |
| | HFRW2 | HWVRQAPGKGLEWVA | 10 |
| | HFRW3 | YADSVKGRFTISADTSKNTAYLQMNSLRAEDTAVYYCAR | 11 |
| | HFRW4 | DYWGQGTLVTVSS | 12 |
| | LFRW1 | SDIQMTQSPSSLSASVGDRVTITCRASQ | 13 |
| | LFRW2 | VAWYQQKPGKAPKLLIY | 14 |
| | LFRW3 | GVPSRFSGSRSGTDFTLTISSLQPEDFATYYCQQ | 15 |
| | LFRW4 | TFGQGTKVEIKRT | 16 |
| | HCDR1 | FYSSYI | 45 |
| | HCDR2 | SISSSSGSTS | 42 |
| | HCDR3 | RSGGGL | 46 |
| | LCDR1 | SVSSA | 4 |
| | LCDR2 | SASSLYS | 5 |
| | LCDR3 | SSSSLI | 38 |
| | VH | EISEVQLVESGGGLVQPGGSLRLSCAASGFNFYSSYIHWV | 47 |
| | | RQAPGKGLEWVASISSSSGSTSYADSVKGRFTISADTSKNT | |
| | | AYLQMNSLRAEDTAVYYCARRSGGGLDYWGQGTLVTVS | |
| | | S | |
| | VL | SDIQMTQSPSSLSASVGDRVTITCRASQSVSSAVAWYQQK | 40 |
| | | PGKAPKLLIYSASSLYSGVPSRFSGSRSGTDFTLTISSLQPE | |
| | | DFATYYCQQSSSSLITFGQGTKVEIKRT | |
| | HFRW1 | EISEVQLVESGGGLVQPGGSLRLSCAASGFN | 9 |
| | HFRW2 | HWVRQAPGKGLEWVA | 10 |
| | HFRW3 | YADSVKGRFTISADTSKNTAYLQMNSLRAEDTAVYYCAR | 11 |
| | HFRW4 | DYWGQGTLVTVSS | 12 |
| | LFRW1 | SDIQMTQSPSSLSASVGDRVTITCRASQ | 13 |
| | LFRW2 | VAWYQQKPGKAPKLLIY | 14 |
| | LFRW3 | GVPSRFSGSRSGTDFTLTISSLQPEDFATYYCQQ | 15 |
| | LFRW4 | TFGQGTKVEIKRT | 16 |
| BOR-C- | HCDR1 | VYYSSI | 35 |
| 64 | HCDR2 | SISSYYGSTY | 48 |
| | HCDR3 | WKWVAM | 49 |
| | LCDR1 | SVSSA | 4 |
| | LCDR2 | SASSLYS | 5 |
| | LCDR3 | DLFMPI | 50 |
| | VH | EISEVQLVESGGGLVQPGGSLRLSCAASGFNVYYSSIHWV | 51 |
| | | RQAPGKGLEWVASISSYYGSTYYADSVKGRFTISADTSKN | |
| | | TAYLQMNSLRAEDTAVYYCARWKWVAMDYWGQGTLV | |
| | | TVSS | |
| | VL | SDIQMTQSPSSLSASVGDRVTITCRASQSVSSAVAWYQQK | 52 |
| | | PGKAPKLLIYSASSLYSGVPSRFSGSRSGTDFTLTISSLQPE | |
| | | DFATYYCQQDLFMPITFGQGTKVEIKRT | |
| | HFRW1 | EISEVQLVESGGGLVQPGGSLRLSCAASGFN | 9 |
| | HFRW2 | HWVRQAPGKGLEWVA | 10 |
| | HFRW3 | YADSVKGRFTISADTSKNTAYLQMNSLRAEDTAVYYCAR | 11 |
| | HFRW4 | DYWGQGTLVTVSS | 12 |
| | LFRW1 | SDIQMTQSPSSLSASVGDRVTITCRASQ | 13 |
| | LFRW2 | VAWYQQKPGKAPKLLIY | 14 |
| | LFRW3 | GVPSRFSGSRSGTDFTLTISSLQPEDFATYYCQQ | 15 |
| | LFRW4 | TFGQGTKVEIKRT | 16 |
| BOR-C- | HCDR1 | FSSSSI | 53 |
| 63 | HCDR2 | SIYSYSGSTY | 54 |
| | HCDR3 | GLYYYLPSYYIYYRSYKFGM | 55 |
| | LCDR1 | SVSSA | 4 |
| | LCDR2 | SASSLYS | 5 |
| | LCDR3 | SSSSLI | 38 |
| | VH | EISEVQLVESGGGLVQPGGSLRLSCAASGFNFSSSSIHWVR | 56 |
| | | QAPGKGLEWVASIYSYSGSTYYADSVKGRFTISADTSKNT | |
| | | AYLQMNSLRAEDTAVYYCARGLYYYLPSYYIYYRSYKFG | |
| | | MDYWGQGTLVTVSS | |
| | VL | SDIQMTQSPSSLSASVGDRVTITCRASQSVSSAVAWYQQK | 40 |
| | | PGKAPKLLIYSASSLYSGVPSRFSGSRSGTDFTLTISSLQPE | |
| | | DFATYYCQQSSSSLITFGQGTKVEIKRT | |
| | HFRW1 | EISEVQLVESGGGLVQPGGSLRLSCAASGFN | 9 |
| | HFRW2 | HWVRQAPGKGLEWVA | 10 |
| | HFRW3 | YADSVKGRFTISADTSKNTAYLQMNSLRAEDTAVYYCAR | 11 |
| | HFRW4 | DYWGQGTLVTVSS | 12 |
| | LFRW1 | SDIQMTQSPSSLSASVGDRVTITCRASQ | 13 |
| | LFRW2 | VAWYQQKPGKAPKLLIY | 14 |
| | LFRW3 | GVPSRFSGSRSGTDFTLTISSLQPEDFATYYCQQ | 15 |
| | LFRW4 | TFGQGTKVEIKRT | 16 |
| BOR-N- | HCDR1 | FSYSSI | 57 |
| 48 | HCDR2 | SIYPYSGSTY | 58 |
| | HCDR3 | KQVSGWWWYYGF | 59 |
| | LCDR1 | SVSSA | 4 |
| | LCDR2 | SASSLYS | 5 |
| | LCDR3 | SYWWPI | 60 |
| | VH | EISEVQLVESGGGLVQPGGSLRLSCAASGFNFSYSSIHWVR | 61 |
| | | QAPGKGLEWVASIYPYSGSTYYADSVKGRFTISADTSKNT | |
| | | AYLQMNSLRAEDTAVYYCARKQVSGWWWYYGFDYWG | |
| | | QGTLVTVSS | |
| | VL | SDIQMTQSPSSLSASVGDRVTITCRASQSVSSAVAWYQQK | 62 |
| | | PGKAPKLLIYSASSLYSGVPSRFSGSRSGTDFTLTISSLQPE | |
| | | DFATYYCQQSYWWPITFGQGTKVEIKRT | |
| | HFRW1 | EISEVQLVESGGGLVQPGGSLRLSCAASGFN | 9 |
| | HFRW2 | HWVRQAPGKGLEWVA | 10 |
| | HFRW3 | YADSVKGRFTISADTSKNTAYLQMNSLRAEDTAVYYCAR | 11 |
| | HFRW4 | DYWGQGTLVTVSS | 12 |
| | LFRW1 | SDIQMTQSPSSLSASVGDRVTITCRASQ | 13 |
| | LFRW2 | VAWYQQKPGKAPKLLIY | 14 |
| | LFRW3 | GVPSRFSGSRSGTDFTLTISSLQPEDFATYYCQQ | 15 |
| | LFRW4 | TFGQGTKVEIKRT | 16 |
| BOR-N- | HCDR1 | VSSYSI | 41 |
| 49 | HCDR2 | SIYSSSSSTY | 63 |
| | HCDR3 | RYYHYRYPLFWGWYGM | 64 |
| | LCDR1 | SVSSA | 4 |
| | LCDR2 | SASSLYS | 5 |
| | LCDR3 | SFWWPI | 65 |
| | VH | EISEVQLVESGGGLVQPGGSLRLSCAASGFNVSSYSIHWV | 66 |
| | | RQAPGKGLEWVASIYSSSSSTYYADSVKGRFTISADTSKN | |
| | | TAYLQMNSLRAEDTAVYYCARRYYHYRYPLFWGWYGM | |
| | | DYWGQGTLVTVSS | |
| | VL | SDIQMTQSPSSLSASVGDRVTITCRASQSVSSAVAWYQQK | 67 |
| | | PGKAPKLLIYSASSLYSGVPSRFSGSRSGTDFTLTISSLQPE | |
| | | DFATYYCQQSFWWPITFGQGTKVEIKRT | |
| | HFRW1 | EISEVQLVESGGGLVQPGGSLRLSCAASGEN | 9 |
| | HFRW2 | HWVRQAPGKGLEWVA | 10 |
| | HFRW3 | YADSVKGRFTISADTSKNTAYLQMNSLRAEDTAVYYCAR | 11 |
| | HFRW4 | DYWGQGTLVTVSS | 12 |
| | LFRW1 | SDIQMTQSPSSLSASVGDRVTITCRASQ | 13 |
| | LFRW2 | VAWYQQKPGKAPKLLIY | 14 |
| | LFRW3 | GVPSRFSGSRSGTDFTLTISSLQPEDFATYYCQQ | 15 |
| | LFRW4 | TFGQGTKVEIKRT | 16 |
| BOR-N- | HCDR1 | VYSYSI | 29 |
| 50 | HCDR2 | SIYSYSGYTY | 68 |
| | HCDR3 | STYPHVYLYFGL | 69 |
| | LCDR1 | SVSSA | 4 |
| | LCDR2 | SASSLYS | 5 |
| | LCDR3 | SYYWPV | 70 |
| | VH | EISEVQLVESGGGLVQPGGSLRLSCAASGFNVYSYSIHWV | 71 |
| | | RQAPGKGLEWVASIYSYSGYTYYADSVKGRFTISADTSKN | |
| | | TAYLQMNSLRAEDTAVYYCARSTYPHVYLYFGLDYWGQ | |
| | | GTLVTVSS | |
| | VL | SDIQMTQSPSSLSASVGDRVTITCRASQSVSSAVAWYQQK | 72 |
| | | PGKAPKLLIYSASSLYSGVPSRFSGSRSGTDFTLTISSLQPE | |
| | | DFATYYCQQSYYWPVTFGQGTKVEIKRT | |
| | HFRW1 | EISEVQLVESGGGLVQPGGSLRLSCAASGFN | 9 |
| | HFRW2 | HWVRQAPGKGLEWVA | 10 |
| | HFRW3 | YADSVKGRFTISADTSKNTAYLQMNSLRAEDTAVYYCAR | 11 |
| | HFRW4 | DYWGQGTLVTVSS | 12 |
| | LFRW1 | SDIQMTQSPSSLSASVGDRVTITCRASQ | 13 |
| | LFRW2 | VAWYQQKPGKAPKLLIY | 14 |
| | LFRW3 | GVPSRFSGSRSGTDFTLTISSLQPEDFATYYCQQ | 15 |
| | LFRW4 | TFGQGTKVEIKRT | 16 |
| BOR-C- | HCDR1 | ISSSSI | 73 |
| 56 | HCDR2 | YISSYSGSTY | 74 |
| | HCDR3 | YYHSPLWYYSYSGM | 75 |
| | LCDR1 | SVSSA | 4 |
| | LCDR2 | SASSLYS | 5 |
| | LCDR3 | DYSWSPI | 76 |
| | VH | EISEVQLVESGGGLVQPGGSLRLSCAASGFNISSSSIHWVR | 77 |
| | | QAPGKGLEWVAYISSYSGSTYYADSVKGRFTISADTSKNT | |
| | | AYLQMNSLRAEDTAVYYCARYYHSPLWYYSYSGMDYW | |
| | | GQGTLVTVSS | |
| | VL | SDIQMTQSPSSLSASVGDRVTITCRASQSVSSAVAWYQQK | 78 |
| | | PGKAPKLLIYSASSLYSGVPSRFSGSRSGTDFTLTISSLQPE | |
| | | DFATYYCQQDYSWSPITFGQGTKVEIKRT | |
| | HFRW1 | EISEVQLVESGGGLVQPGGSLRLSCAASGFN | 9 |
| | HFRW2 | HWVRQAPGKGLEWVA | 10 |
| | HFRW3 | YADSVKGRFTISADTSKNTAYLQMNSLRAEDTAVYYCAR | 11 |
| | HFRW4 | DYWGQGTLVTVSS | 12 |
| | LFRW1 | SDIQMTQSPSSLSASVGDRVTITCRASQ | 13 |
| | LFRW2 | VAWYQQKPGKAPKLLIY | 14 |
| | LFRW3 | GVPSRFSGSRSGTDFTLTISSLQPEDFATYYCQQ | 15 |
| | LFRW4 | TFGQGTKVEIKRT | 16 |
| BOR-C- | HCDR1 | ISSSSI | 73 |
| 57 | HCDR2 | SISSYYSYTY | 79 |
| | HCDR3 | GAWYGYLGYYAYYYYAL | 80 |
| | LCDR1 | SVSSA | 4 |
| | LCDR2 | SASSLYS | 5 |
| | LCDR3 | MGYETLI | 81 |
| | VH | EISEVQLVESGGGLVQPGGSLRLSCAASGFNISSSSIHWVR | 82 |
| | | QAPGKGLEWVASISSYYSYTYYADSVKGRFTISADTSKNT | |
| | | AYLQMNSLRAEDTAVYYCARGAWYGYLGYYAYYYYAL | |
| | | DYWGQGTLVTVSS | |
| | VL | SDIQMTQSPSSLSASVGDRVTITCRASQSVSSAVAWYQQK | 83 |
| | | PGKAPKLLIYSASSLYSGVPSRFSGSRSGTDFTLTISSLQPE | |
| | | DFATYYCQQMGYETLITFGQGTKVEIKRT | |
| | HFRW1 | EISEVQLVESGGGLVQPGGSLRLSCAASGEN | 9 |
| | HFRW2 | HWVRQAPGKGLEWVA | 10 |
| | HFRW3 | YADSVKGRFTISADTSKNTAYLQMNSLRAEDTAVYYCAR | 11 |
| | HFRW4 | DYWGQGTLVTVSS | 12 |
| | LFRW1 | SDIQMTQSPSSLSASVGDRVTITCRASQ | 13 |
| | LFRW2 | VAWYQQKPGKAPKLLIY | 14 |
| | LFRW3 | GVPSRFSGSRSGTDFTLTISSLQPEDFATYYCQQ | 15 |
| | LFRW4 | TFGQGTKVEIKRT | 16 |
| BOR-N- | HCDR1 | FSSSSI | 53 |
| 65 | HCDR2 | SISSSSGSTS | 42 |
| | HCDR3 | ESSQTKKYWYWVVQWYTAM | 84 |
| | LCDR1 | SVSSA | 4 |
| | LCDR2 | SASSLYS | 5 |
| | LCDR3 | YSQWLI | 85 |
| | VH | EISEVQLVESGGGLVQPGGSLRLSCAASGFNFSSSSIHWVR | 86 |
| | | QAPGKGLEWVASISSSSGSTSYADSVKGRFTISADTSKNTA | |
| | | YLQMNSLRAEDTAVYYCARESSQTKKYWYWVVQWYTA | |
| | | MDYWGQGTLVTVSS | |
| | VL | SDIQMTQSPSSLSASVGDRVTITCRASQSVSSAVAWYQQK | 87 |
| | | PGKAPKLLIYSASSLYSGVPSRFSGSRSGTDFTLTISSLQPE | |
| | | DFATYYCQQYSQWLITFGQGTKVEIKRT | |
| | HFRW1 | EISEVQLVESGGGLVQPGGSLRLSCAASGFN | 9 |
| | HFRW2 | HWVRQAPGKGLEWVA | 10 |
| | HFRW3 | YADSVKGRFTISADTSKNTAYLQMNSLRAEDTAVYYCAR | 11 |
| | HFRW4 | DYWGQGTLVTVSS | 12 |
| | LFRW1 | SDIQMTQSPSSLSASVGDRVTITCRASQ | 13 |
| | LFRW2 | VAWYQQKPGKAPKLLIY | 14 |
| | LFRW3 | GVPSRFSGSRSGTDFTLTISSLQPEDFATYYCQQ | 15 |
| | LFRW4 | TFGQGTKVEIKRT | 16 |
| BOR-C- | HCDR1 | VSSYSI | 41 |
| 58 | HCDR2 | SISPYYGYTS | 88 |
| | HCDR3 | HYYYGFSWFSSGYAM | 89 |
| | LCDR1 | SVSSA | 4 |
| | LCDR2 | SASSLYS | 5 |
| | LCDR3 | GSSSPI | 90 |
| | VH | EISEVQLVESGGGLVQPGGSLRLSCAASGFNVSSYSIHWV | 91 |
| | | RQAPGKGLEWVASISPYYGYTSYADSVKGRFTISADTSKN | |
| | | TAYLQMNSLRAEDTAVYYCARHYYYGFSWFSSGYAMDY | |
| | | WGQGTLVTVSS | |
| | VL | SDIQMTQSPSSLSASVGDRVTITCRASQSVSSAVAWYQQK | 92 |
| | | PGKAPKLLIYSASSLYSGVPSRFSGSRSGTDFTLTISSLQPE | |
| | | DFATYYCQQGSSSPITFGQGTKVEIKRT | |
| | HFRW1 | EISEVQLVESGGGLVQPGGSLRLSCAASGFN | 9 |
| | HFRW2 | HWVRQAPGKGLEWVA | 10 |
| | HFRW3 | YADSVKGRFTISADTSKNTAYLQMNSLRAEDTAVYYCAR | 11 |
| | HFRW4 | DYWGQGTLVTVSS | 12 |
| | LFRW1 | SDIQMTQSPSSLSASVGDRVTITCRASQ | 13 |
| | LFRW2 | VAWYQQKPGKAPKLLIY | 14 |
| | LFRW3 | GVPSRFSGSRSGTDFTLTISSLQPEDFATYYCQQ | 15 |
| | LFRW4 | TFGQGTKVEIKRT | 16 |
| | Light | VAAPSVFIFPPSDSQLKSGTASVVCLLNNFYPREAKVQWK | 122 |
| | Chain Fab | VDNALQSGNSQESVTEQDSKDSTYSLSSTLTLSKADYEKH | |
| | Constant | KVYACEVTHQGLSSPVTKSFNRGEC | |
| | region | | |
| | Heavy | ASTKGPSVFPLAPSSKSTSGGTAALGCLVKDYFPEPVTVS | 123 |
| | Chain Fab | WNSGALTSGVHTFPAVLQSSGLYSLSSVVTVPSSSLGTQT | |
| | Constant | YICNVNHKPSNTKVDKKVEPKSCDKTHT | |
| | region | | |

TABLE 2
Nucleic Acid Sequences

| | | | SEQ |
|---|---|---|---|
| | | | ID |
| Clone | Region | Sequence | NO: |
| BOR- | Fab | GAGATCTCCGAGGTTCAGCTGGTGGAGTCTGGCGGTGGCCT | 93 |
| N-54 | Heavy | GGTGCAGCCAGGGGGCTCACTCCGTTTGTCCTGTGCAGCTTC | |
| | Chain | TGGCTTCAACATCTATTATTCTTCTATACACTGGGTGCGTCA | |
| | | GGCCCCGGGTAAGGGCCTGGAATGGGTTGCATCTATTTATC | |
| | | CTTATTATGGCTCTACTTCTTATGCCGATAGCGTCAAGGGCC | |
| | | GTTTCACTATAAGCGCAGACACATCCAAAAACACAGCCTAC | |
| | | CTACAAATGAACAGCTTAAGAGCTGAGGACACTGCCGTCTA | |
| | | TTATTGTGCTCGCAAATGGTACTACGGTGACGTTGGTATGGA | |
| | | CTACTGGGGTCAAGGAACCCTGGTCACCGTCTCCTCGGCCTC | |
| | | CACCAAGGGTCCATCGGTCTTCCCCCTGGCACCCTCCTCCAA | |
| | | GAGCACCTCTGGGGGCACAGCGGCCCTGGGCTGCCTGGTCA | |
| | | AGGACTACTTCCCCGAACCGGTGACGGTGTCGTGGAACTCA | |
| | | GGCGCCCTGACCAGCGGCGTGCACACCTTCCCGGCTGTCCT | |
| | | ACAGTCCTCAGGACTCTACTCCCTCAGCAGCGTGGTGACCG | |
| | | TGCCCTCCAGCAGCTTGGGCACCCAGACCTACATCTGCAAC | |
| | | GTGAATCACAAGCCCAGCAACACCAAGGTCGACAAGAAAG | |
| | | TTGAGCCCAAATCTTGTGACAAAACTCACACATAA | |
| | Fab | TCCGATATCCAGATGACCCAGTCCCCGAGCTCCCTGTCCGCC | 94 |
| | Light | TCTGTGGGCGATAGGGTCACCATCACCTGCCGTGCCAGTCA | |
| | Chain | GTCCGTGTCCAGCGCTGTAGCCTGGTATCAACAGAAACCAG | |
| | | GAAAAGCTCCGAAGCTTCTGATTTACTCGGCATCCAGCCTCT | |
| | | ACTCTGGAGTCCCTTCTCGCTTCTCTGGTAGCCGTTCCGGGA | |
| | | CGGATTTCACTCTGACCATCAGCAGTCTGCAGCCGGAAGAC | |
| | | TTCGCAACTTATTACTGTCAGCAATCTGAATACCGTCTGATC | |
| | | ACGTTCGGACAGGGTACCAAGGTGGAGATCAAACGAACTGT | |
| | | GGCTGCACCATCTGTCTTCATCTTCCCGCCATCTGATTCACA | |
| | | GTTGAAATCTGGAACTGCCTCTGTTGTGTGCCTGCTGAATAA | |
| | | CTTCTATCCCAGAGAGGCCAAAGTACAGTGGAAGGTGGATA | |
| | | ACGCCCTCCAATCGGGTAACTCCCAGGAGAGTGTCACAGAG | |
| | | CAGGACAGCAAGGACAGCACCTACAGCCTCAGCAGCACCCT | |
| | | GACGCTGAGCAAAGCAGACTACGAAAAACATAAAGTCTAC | |
| | | GCCTGCGAAGTCACCCATCAGGGCCTGAGCTCGCCCGTCAC | |
| | | AAAGAGCTTCAACAGGGGAGAGTGTTAA | |
| BOR- | Fab | GAGATCTCCGAGGTTCAGCTGGTGGAGTCTGGCGGTGGCCT | 95 |
| C-59 | Heavy | GGTGCAGCCAGGGGGCTCACTCCGTTTGTCCTGTGCAGCTTC | |
| | Chain | TGGCTTCAACCTCTCTTCTTCTTATATACACTGGGTGCGTCA | |
| | | GGCCCCGGGTAAGGGCCTGGAATGGGTTGCATATATTTATT | |
| | | CTTCTTCTGGCTCTACTTATTATGCCGATAGCGTCAAGGGCC | |
| | | GTTTCACTATAAGCGCAGACACATCCAAAAACACAGCCTAC | |
| | | CTACAAATGAACAGCTTAAGAGCTGAGGACACTGCCGTCTA | |
| | | TTATTGTGCTCGCTGGCCGACTTACTACTACTGGCTGGTTGG | |
| | | TTGGATGTACGACGTTGGTGCTTTTGACTACTGGGGTCAAGG | |
| | | AACCCTGGTCACCGTCTCCTCGGCCTCCACCAAGGGTCCATC | |
| | | GGTCTTCCCCCTGGCACCCTCCTCCAAGAGCACCTCTGGGGG | |
| | | CACAGCGGCCCTGGGCTGCCTGGTCAAGGACTACTTCCCCG | |
| | | AACCGGTGACGGTGTCGTGGAACTCAGGCGCCCTGACCAGC | |
| | | GGCGTGCACACCTTCCCGGCTGTCCTACAGTCCTCAGGACTC | |
| | | TACTCCCTCAGCAGCGTGGTGACCGTGCCCTCCAGCAGCTTG | |
| | | GGCACCCAGACCTACATCTGCAACGTGAATCACAAGCCCAG | |
| | | CAACACCAAGGTCGACAAGAAAGTTGAGCCCAAATCTTGTG | |
| | | ACAAAACTCACACATAA | |
| | Fab | TCCGATATCCAGATGACCCAGTCCCCGAGCTCCCTGTCCGCC | 96 |
| | Light | TCTGTGGGCGATAGGGTCACCATCACCTGCCGTGCCAGTCA | |
| | Chain | GTCCGTGTCCAGCGCTGTAGCCTGGTATCAACAGAAACCAG | |
| | | GAAAAGCTCCGAAGCTTCTGATTTACTCGGCATCCAGCCTCT | |
| | | ACTCTGGAGTCCCTTCTCGCTTCTCTGGTAGCCGTTCCGGGA | |
| | | CGGATTTCACTCTGACCATCAGCAGTCTGCAGCCGGAAGAC | |
| | | TTCGCAACTTATTACTGTCAGCAATCTTCTTCTGAATACCCG | |
| | | GTCACGTTCGGACAGGGTACCAAGGTGGAGATCAAACGAAC | |
| | | TGTGGCTGCACCATCTGTCTTCATCTTCCCGCCATCTGATTC | |
| | | ACAGTTGAAATCTGGAACTGCCTCTGTTGTGTGCCTGCTGAA | |
| | | TAACTTCTATCCCAGAGAGGCCAAAGTACAGTGGAAGGTGG | |
| | | ATAACGCCCTCCAATCGGGTAACTCCCAGGAGAGTGTCACA | |
| | | GAGCAGGACAGCAAGGACAGCACCTACAGCCTCAGCAGCA | |
| | | CCCTGACGCTGAGCAAAGCAGACTACGAAAAACATAAAGTC | |
| | | TACGCCTGCGAAGTCACCCATCAGGGCCTGAGCTCGCCCGT | |
| | | CACAAAGAGCTTCAACAGGGGAGAGTGTTAA | |
| BOR- | Fab | GAGATCTCCGAGGTTCAGCTGGTGGAGTCTGGCGGTGGCCT | 97 |
| C-60 | Heavy | GGTGCAGCCAGGGGGCTCACTCCGTTTGTCCTGTGCAGCTTC | |
| | Chain | TGGCTTCAACTTCTCTTATTCTTATATACACTGGGTGCGTCA | |
| | | GGCCCCGGGTAAGGGCCTGGAATGGGTTGCATATATTTCTT | |
| | | CTTCTTCTGGCTATACTTATTATGCCGATAGCGTCAAGGGCC | |
| | | GTTTCACTATAAGCGCAGACACATCCAAAAACACAGCCTAC | |
| | | CTACAAATGAACAGCTTAAGAGCTGAGGACACTGCCGTCTA | |
| | | TTATTGTGCTCGCTCTGGTCTGTACTCTCATGGTGTTTGGCA | |
| | | GAACTGGTACGGTTACATGGGTTTTGACTACTGGGGTCAAG | |
| | | GAACCCTGGTCACCGTCTCCTCGGCCTCCACCAAGGGTCCAT | |
| | | CGGTCTTCCCCCTGGCACCCTCCTCCAAGAGCACCTCTGGGG | |
| | | GCACAGCGGCCCTGGGCTGCCTGGTCAAGGACTACTTCCCC | |
| | | GAACCGGTGACGGTGTCGTGGAACTCAGGCGCCCTGACCAG | |
| | | CGGCGTGCACACCTTCCCGGCTGTCCTACAGTCCTCAGGACT | |
| | | CTACTCCCTCAGCAGCGTGGTGACCGTGCCCTCCAGCAGCTT | |
| | | GGGCACCCAGACCTACATCTGCAACGTGAATCACAAGCCCA | |
| | | GCAACACCAAGGTCGACAAGAAAGTTGAGCCCAAATCTTGT | |
| | | GACAAAACTCACACATAA | |
| | Fab | TCCGATATCCAGATGACCCAGTCCCCGAGCTCCCTGTCCGCC | 98 |
| | Light | TCTGTGGGCGATAGGGTCACCATCACCTGCCGTGCCAGTCA | |
| | Chain | GTCCGTGTCCAGCGCTGTAGCCTGGTATCAACAGAAACCAG | |
| | | GAAAAGCTCCGAAGCTTCTGATTTACTCGGCATCCAGCCTCT | |
| | | ACTCTGGAGTCCCTTCTCGCTTCTCTGGTAGCCGTTCCGGGA | |
| | | CGGATTTCACTCTGACCATCAGCAGTCTGCAGCCGGAAGAC | |
| | | TTCGCAACTTATTACTGTCAGCAATCTCTGCAGTGGCCGATC | |
| | | ACGTTCGGACAGGGTACCAAGGTGGAGATCAAACGAACTGT | |
| | | GGCTGCACCATCTGTCTTCATCTTCCCGCCATCTGATTCACA | |
| | | GTTGAAATCTGGAACTGCCTCTGTTGTGTGCCTGCTGAATAA | |
| | | CTTCTATCCCAGAGAGGCCAAAGTACAGTGGAAGGTGGATA | |
| | | ACGCCCTCCAATCGGGTAACTCCCAGGAGAGTGTCACAGAG | |
| | | CAGGACAGCAAGGACAGCACCTACAGCCTCAGCAGCACCCT | |
| | | GACGCTGAGCAAAGCAGACTACGAAAAACATAAAGTCTAC | |
| | | GCCTGCGAAGTCACCCATCAGGGCCTGAGCTCGCCCGTCAC | |
| | | AAAGAGCTTCAACAGGGGAGAGTGTTAA | |
| BOR- | Fab | GAGATCTCCGAGGTTCAGCTGGTGGAGTCTGGCGGTGGCCT | 99 |
| N-51 | Heavy | GGTGCAGCCAGGGGGCTCACTCCGTTTGTCCTGTGCAGCTTC | |
| | Chain | TGGCTTCAACGTCTATTCTTATTCTATACACTGGGTGCGTCA | |
| | | GGCCCCGGGTAAGGGCCTGGAATGGGTTGCATCTATTTATT | |
| | | CTTCTTATAGCTATACTTCTTATGCCGATAGCGTCAAGGGCC | |
| | | GTTTCACTATAAGCGCAGACACATCCAAAAACACAGCCTAC | |
| | | CTACAAATGAACAGCTTAAGAGCTGAGGACACTGCCGTCTA | |
| | | TTATTGTGCTCGCAAATACCATTTCAAACCGACTTACTGGGG | |
| | | TGCTATGGACTACTGGGGTCAAGGAACCCTGGTCACCGTCT | |
| | | CCTCGGCCTCCACCAAGGGTCCATCGGTCTTCCCCCTGGCAC | |
| | | CCTCCTCCAAGAGCACCTCTGGGGGCACAGCGGCCCTGGGC | |
| | | TGCCTGGTCAAGGACTACTTCCCCGAACCGGTGACGGTGTC | |
| | | GTGGAACTCAGGCGCCCTGACCAGCGGCGTGCACACCTTCC | |
| | | CGGCTGTCCTACAGTCCTCAGGACTCTACTCCCTCAGCAGCG | |
| | | TGGTGACCGTGCCCTCCAGCAGCTTGGGCACCCAGACCTAC | |
| | | ATCTGCAACGTGAATCACAAGCCCAGCAACACCAAGGTCGA | |
| | | CAAGAAAGTTGAGCCCAAATCTTGTGACAAAACTCACACAT | |
| | | AA | |
| | Fab | TCCGATATCCAGATGACCCAGTCCCCGAGCTCCCTGTCCGCC | 100 |
| | Light | TCTGTGGGCGATAGGGTCACCATCACCTGCCGTGCCAGTCA | |
| | Chain | GTCCGTGTCCAGCGCTGTAGCCTGGTATCAACAGAAACCAG | |
| | | GAAAAGCTCCGAAGCTTCTGATTTACTCGGCATCCAGCCTCT | |
| | | ACTCTGGAGTCCCTTCTCGCTTCTCTGGTAGCCGTTCCGGGA | |
| | | CGGATTTCACTCTGACCATCAGCAGTCTGCAGCCGGAAGAC | |
| | | TTCGCAACTTATTACTGTCAGCAATCTTCTCTGTACTACCCG | |
| | | CTCACGTTCGGACAGGGTACCAAGGTGGAGATCAAACGAAC | |
| | | TGTGGCTGCACCATCTGTCTTCATCTTCCCGCCATCTGATTC | |
| | | ACAGTTGAAATCTGGAACTGCCTCTGTTGTGTGCCTGCTGAA | |
| | | TAACTTCTATCCCAGAGAGGCCAAAGTACAGTGGAAGGTGG | |
| | | ATAACGCCCTCCAATCGGGTAACTCCCAGGAGAGTGTCACA | |
| | | GAGCAGGACAGCAAGGACAGCACCTACAGCCTCAGCAGCA | |
| | | CCCTGACGCTGAGCAAAGCAGACTACGAAAAACATAAAGTC | |
| | | TACGCCTGCGAAGTCACCCATCAGGGCCTGAGCTCGCCCGT | |
| | | CACAAAGAGCTTCAACAGGGGAGAGTGTTAA | |
| BOR- | Fab | GAGATCTCCGAGGTTCAGCTGGTGGAGTCTGGCGGTGGCCT | 101 |
| N-55 | Heavy | GGTGCAGCCAGGGGGCTCACTCCGTTTGTCCTGTGCAGCTTC | |
| | Chain | TGGCTTCAACGTCTATTATTCTTCTATACACTGGGTGCGTCA | |
| | | GGCCCCGGGTAAGGGCCTGGAATGGGTTGCATCTATTTCTC | |
| | | CTTATTATGGCTCTACTTCTTATGCCGATAGCGTCAAGGGCC | |
| | | GTTTCACTATAAGCGCAGACACATCCAAAAACACAGCCTAC | |
| | | CTACAAATGAACAGCTTAAGAGCTGAGGACACTGCCGTCTA | |
| | | TTATTGTGCTCGCCGTTGGTACTACACTAAAGTTTTCATGTG | |
| | | GGGTATGGACTACTGGGGTCAAGGAACCCTGGTCACCGTCT | |
| | | CCTCGGCCTCCACCAAGGGTCCATCGGTCTTCCCCCTGGCAC | |
| | | CCTCCTCCAAGAGCACCTCTGGGGGCACAGCGGCCCTGGGC | |
| | | TGCCTGGTCAAGGACTACTTCCCCGAACCGGTGACGGTGTC | |
| | | GTGGAACTCAGGCGCCCTGACCAGCGGCGTGCACACCTTCC | |
| | | CGGCTGTCCTACAGTCCTCAGGACTCTACTCCCTCAGCAGCG | |
| | | TGGTGACCGTGCCCTCCAGCAGCTTGGGCACCCAGACCTAC | |
| | | ATCTGCAACGTGAATCACAAGCCCAGCAACACCAAGGTCGA | |
| | | CAAGAAAGTTGAGCCCAAATCTTGTGACAAAACTCACACAT | |
| | | AA | |
| | Fab | TCCGATATCCAGATGACCCAGTCCCCGAGCTCCCTGTCCGCC | 102 |
| | Light | TCTGTGGGCGATAGGGTCACCATCACCTGCCGTGCCAGTCA | |
| | Chain | GTCCGTGTCCAGCGCTGTAGCCTGGTATCAACAGAAACCAG | |
| | | GAAAAGCTCCGAAGCTTCTGATTTACTCGGCATCCAGCCTCT | |
| | | ACTCTGGAGTCCCTTCTCGCTTCTCTGGTAGCCGTTCCGGGA | |
| | | CGGATTTCACTCTGACCATCAGCAGTCTGCAGCCGGAAGAC | |
| | | TTCGCAACTTATTACTGTCAGCAATCTTCTTCTTCTCTGATCA | |
| | | CGTTCGGACAGGGTACCAAGGTGGAGATCAAACGAACTGTG | |
| | | GCTGCACCATCTGTCTTCATCTTCCCGCCATCTGATTCACAG | |
| | | TTGAAATCTGGAACTGCCTCTGTTGTGTGCCTGCTGAATAAC | |
| | | TTCTATCCCAGAGAGGCCAAAGTACAGTGGAAGGTGGATAA | |
| | | CGCCCTCCAATCGGGTAACTCCCAGGAGAGTGTCACAGAGC | |
| | | AGGACAGCAAGGACAGCACCTACAGCCTCAGCAGCACCCTG | |
| | | ACGCTGAGCAAAGCAGACTACGAAAAACATAAAGTCTACG | |
| | | CCTGCGAAGTCACCCATCAGGGCCTGAGCTCGCCCGTCACA | |
| | | AAGAGCTTCAACAGGGGAGAGTGTTAA | |
| BOR- | Fab | GAGATCTCCGAGGTTCAGCTGGTGGAGTCTGGCGGTGGCCT | 103 |
| C-62 | Heavy | GGTGCAGCCAGGGGGCTCACTCCGTTTGTCCTGTGCAGCTTC | |
| | Chain | TGGCTTCAACGTCTCTTCTTATTCTATACACTGGGTGCGTCA | |
| | | GGCCCCGGGTAAGGGCCTGGAATGGGTTGCATCTATTTCTTC | |
| | | TTCTTCTGGCTCTACTTCTTATGCCGATAGCGTCAAGGGCCG | |
| | | TTTCACTATAAGCGCAGACACATCCAAAAACACAGCCTACC | |
| | | TACAAATGAACAGCTTAAGAGCTGAGGACACTGCCGTCTAT | |
| | | TATTGTGCTCGCGAATCTAACCAGTACTACTACTACTACAAC | |
| | | GTTCTGTGGTGGTACCAGGCTTTTGACTACTGGGGTCAAGG | |
| | | AACCCTGGTCACCGTCTCCTCGGCCTCCACCAAGGGTCCATC | |
| | | GGTCTTCCCCCTGGCACCCTCCTCCAAGAGCACCTCTGGGGG | |
| | | CACAGCGGCCCTGGGCTGCCTGGTCAAGGACTACTTCCCCG | |
| | | AACCGGTGACGGTGTCGTGGAACTCAGGCGCCCTGACCAGC | |
| | | GGCGTGCACACCTTCCCGGCTGTCCTACAGTCCTCAGGACTC | |
| | | TACTCCCTCAGCAGCGTGGTGACCGTGCCCTCCAGCAGCTTG | |
| | | GGCACCCAGACCTACATCTGCAACGTGAATCACAAGCCCAG | |
| | | CAACACCAAGGTCGACAAGAAAGTTGAGCCCAAATCTTGTG | |
| | | ACAAAACTCACACATAA | |
| | Fab | TCCGATATCCAGATGACCCAGTCCCCGAGCTCCCTGTCCGCC | 102 |
| | Light | TCTGTGGGCGATAGGGTCACCATCACCTGCCGTGCCAGTCA | |
| | Chain | GTCCGTGTCCAGCGCTGTAGCCTGGTATCAACAGAAACCAG | |
| | | GAAAAGCTCCGAAGCTTCTGATTTACTCGGCATCCAGCCTCT | |
| | | ACTCTGGAGTCCCTTCTCGCTTCTCTGGTAGCCGTTCCGGGA | |
| | | CGGATTTCACTCTGACCATCAGCAGTCTGCAGCCGGAAGAC | |
| | | TTCGCAACTTATTACTGTCAGCAATCTTCTTCTTCTCTGATCA | |
| | | CGTTCGGACAGGGTACCAAGGTGGAGATCAAACGAACTGTG | |
| | | GCTGCACCATCTGTCTTCATCTTCCCGCCATCTGATTCACAG | |
| | | TTGAAATCTGGAACTGCCTCTGTTGTGTGCCTGCTGAATAAC | |
| | | TTCTATCCCAGAGAGGCCAAAGTACAGTGGAAGGTGGATAA | |
| | | CGCCCTCCAATCGGGTAACTCCCAGGAGAGTGTCACAGAGC | |
| | | AGGACAGCAAGGACAGCACCTACAGCCTCAGCAGCACCCTG | |
| | | ACGCTGAGCAAAGCAGACTACGAAAAACATAAAGTCTACG | |
| | | CCTGCGAAGTCACCCATCAGGGCCTGAGCTCGCCCGTCACA | |
| | | AAGAGCTTCAACAGGGGAGAGTGTTAA | |
| BOR- | Fab | GAGATCTCCGAGGTTCAGCTGGTGGAGTCTGGCGGTGGCCT | 104 |
| C-61 | Heavy | GGTGCAGCCAGGGGGCTCACTCCGTTTGTCCTGTGCAGCTTC | |
| | Chain | TGGCTTCAACTTCTATTCTTCTTATATACACTGGGTGCGTCA | |
| | | GGCCCCGGGTAAGGGCCTGGAATGGGTTGCATCTATTTCTTC | |
| | | TTCTTCTGGCTCTACTTCTTATGCCGATAGCGTCAAGGGCCG | |
| | | TTTCACTATAAGCGCAGACACATCCAAAAACACAGCCTACC | |
| | | TACAAATGAACAGCTTAAGAGCTGAGGACACTGCCGTCTAT | |
| | | TATTGTGCTCGCCGTTCTGGTGGTGGTTTGGACTACTGGGGT | |
| | | CAAGGAACCCTGGTCACCGTCTCCTCGGCCTCCACCAAGGG | |
| | | TCCATCGGTCTTCCCCCTGGCACCCTCCTCCAAGAGCACCTC | |
| | | TGGGGGCACAGCGGCCCTGGGCTGCCTGGTCAAGGACTACT | |
| | | TCCCCGAACCGGTGACGGTGTCGTGGAACTCAGGCGCCCTG | |
| | | ACCAGCGGCGTGCACACCTTCCCGGCTGTCCTACAGTCCTCA | |
| | | GGACTCTACTCCCTCAGCAGCGTGGTGACCGTGCCCTCCAG | |
| | | CAGCTTGGGCACCCAGACCTACATCTGCAACGTGAATCACA | |
| | | AGCCCAGCAACACCAAGGTCGACAAGAAAGTTGAGCCCAA | |
| | | ATCTTGTGACAAAACTCACACATAA | |
| | Fab | TCCGATATCCAGATGACCCAGTCCCCGAGCTCCCTGTCCGCC | 102 |
| | Light | TCTGTGGGCGATAGGGTCACCATCACCTGCCGTGCCAGTCA | |
| | Chain | GTCCGTGTCCAGCGCTGTAGCCTGGTATCAACAGAAACCAG | |
| | | GAAAAGCTCCGAAGCTTCTGATTTACTCGGCATCCAGCCTCT | |
| | | ACTCTGGAGTCCCTTCTCGCTTCTCTGGTAGCCGTTCCGGGA | |
| | | CGGATTTCACTCTGACCATCAGCAGTCTGCAGCCGGAAGAC | |
| | | TTCGCAACTTATTACTGTCAGCAATCTTCTTCTTCTCTGATCA | |
| | | CGTTCGGACAGGGTACCAAGGTGGAGATCAAACGAACTGTG | |
| | | GCTGCACCATCTGTCTTCATCTTCCCGCCATCTGATTCACAG | |
| | | TTGAAATCTGGAACTGCCTCTGTTGTGTGCCTGCTGAATAAC | |
| | | TTCTATCCCAGAGAGGCCAAAGTACAGTGGAAGGTGGATAA | |
| | | CGCCCTCCAATCGGGTAACTCCCAGGAGAGTGTCACAGAGC | |
| | | AGGACAGCAAGGACAGCACCTACAGCCTCAGCAGCACCCTG | |
| | | ACGCTGAGCAAAGCAGACTACGAAAAACATAAAGTCTACG | |
| | | CCTGCGAAGTCACCCATCAGGGCCTGAGCTCGCCCGTCACA | |
| | | AAGAGCTTCAACAGGGGAGAGTGTTAA | |
| BOR- | Fab | GAGATCTCCGAGGTTCAGCTGGTGGAGTCTGGCGGTGGCCT | 105 |
| C-64 | Heavy | GGTGCAGCCAGGGGGCTCACTCCGTTTGTCCTGTGCAGCTTC | |
| | Chain | TGGCTTCAACGTCTATTATTCTTCTATACACTGGGTGCGTCA | |
| | | GGCCCCGGGTAAGGGCCTGGAATGGGTTGCATCTATTTCTTC | |
| | | TTATTATGGCTCTACTTATTATGCCGATAGCGTCAAGGGCCG | |
| | | TTTCACTATAAGCGCAGACACATCCAAAAACACAGCCTACC | |
| | | TACAAATGAACAGCTTAAGAGCTGAGGACACTGCCGTCTAT | |
| | | TATTGTGCTCGCTGGAAATGGGTTGCTATGGACTACTGGGGT | |
| | | CAAGGAACCCTGGTCACCGTCTCCTCGGCCTCCACCAAGGG | |
| | | TCCATCGGTCTTCCCCCTGGCACCCTCCTCCAAGAGCACCTC | |
| | | TGGGGGCACAGCGGCCCTGGGCTGCCTGGTCAAGGACTACT | |
| | | TCCCCGAACCGGTGACGGTGTCGTGGAACTCAGGCGCCCTG | |
| | | ACCAGCGGCGTGCACACCTTCCCGGCTGTCCTACAGTCCTCA | |
| | | GGACTCTACTCCCTCAGCAGCGTGGTGACCGTGCCCTCCAG | |
| | | CAGCTTGGGCACCCAGACCTACATCTGCAACGTGAATCACA | |
| | | AGCCCAGCAACACCAAGGTCGACAAGAAAGTTGAGCCCAA | |
| | | ATCTTGTGACAAAACTCACACATAA | |
| | Fab | TCCGATATCCAGATGACCCAGTCCCCGAGCTCCCTGTCCGCC | 106 |
| | Light | TCTGTGGGCGATAGGGTCACCATCACCTGCCGTGCCAGTCA | |
| | Chain | GTCCGTGTCCAGCGCTGTAGCCTGGTATCAACAGAAACCAG | |
| | | GAAAAGCTCCGAAGCTTCTGATTTACTCGGCATCCAGCCTCT | |
| | | ACTCTGGAGTCCCTTCTCGCTTCTCTGGTAGCCGTTCCGGGA | |
| | | CGGATTTCACTCTGACCATCAGCAGTCTGCAGCCGGAAGAC | |
| | | TTCGCAACTTATTACTGTCAGCAAGACCTGTTCATGCCGATC | |
| | | ACGTTCGGACAGGGTACCAAGGTGGAGATCAAACGAACTGT | |
| | | GGCTGCACCATCTGTCTTCATCTTCCCGCCATCTGATTCACA | |
| | | GTTGAAATCTGGAACTGCCTCTGTTGTGTGCCTGCTGAATAA | |
| | | CTTCTATCCCAGAGAGGCCAAAGTACAGTGGAAGGTGGATA | |
| | | ACGCCCTCCAATCGGGTAACTCCCAGGAGAGTGTCACAGAG | |
| | | CAGGACAGCAAGGACAGCACCTACAGCCTCAGCAGCACCCT | |
| | | GACGCTGAGCAAAGCAGACTACGAAAAACATAAAGTCTAC | |
| | | GCCTGCGAAGTCACCCATCAGGGCCTGAGCTCGCCCGTCAC | |
| | | AAAGAGCTTCAACAGGGGAGAGTGTTAA | |
| BOR- | Fab | GAGATCTCCGAGGTTCAGCTGGTGGAGTCTGGCGGTGGCCT | 107 |
| C-63 | Heavy | GGTGCAGCCAGGGGGCTCACTCCGTTTGTCCTGTGCAGCTTC | |
| | Chain | TGGCTTCAACTTTTCTTCTTCTTCTATACACTGGGTGCGTCAG | |
| | | GCCCCGGGTAAGGGCCTGGAATGGGTTGCATCTATTTATTCT | |
| | | TATTCTGGCTCTACTTATTATGCCGATAGCGTCAAGGGCCGT | |
| | | TTCACTATAAGCGCAGACACATCCAAAAACACAGCCTACCT | |
| | | ACAAATGAACAGCTTAAGAGCTGAGGACACTGCCGTCTATT | |
| | | ATTGTGCTCGCGGTCTGTACTACTACCTGCCGTCTTACTACA | |
| | | TCTACTACCGTTCTTACAAATTCGGTATGGACTACTGGGGTC | |
| | | AAGGAACCCTGGTCACCGTCTCCTCGGCCTCCACCAAGGGT | |
| | | CCATCGGTCTTCCCCCTGGCACCCTCCTCCAAGAGCACCTCT | |
| | | GGGGGCACAGCGGCCCTGGGCTGCCTGGTCAAGGACTACTT | |
| | | CCCCGAACCGGTGACGGTGTCGTGGAACTCAGGCGCCCTGA | |
| | | CCAGCGGCGTGCACACCTTCCCGGCTGTCCTACAGTCCTCAG | |
| | | GACTCTACTCCCTCAGCAGCGTGGTGACCGTGCCCTCCAGC | |
| | | AGCTTGGGCACCCAGACCTACATCTGCAACGTGAATCACAA | |
| | | GCCCAGCAACACCAAGGTCGACAAGAAAGTTGAGCCCAAA | |
| | | TCTTGTGACAAAACTCACACATAA | |
| | Fab | TCCGATATCCAGATGACCCAGTCCCCGAGCTCCCTGTCCGCC | 102 |
| | Light | TCTGTGGGCGATAGGGTCACCATCACCTGCCGTGCCAGTCA | |
| | Chain | GTCCGTGTCCAGCGCTGTAGCCTGGTATCAACAGAAACCAG | |
| | | GAAAAGCTCCGAAGCTTCTGATTTACTCGGCATCCAGCCTCT | |
| | | ACTCTGGAGTCCCTTCTCGCTTCTCTGGTAGCCGTTCCGGGA | |
| | | CGGATTTCACTCTGACCATCAGCAGTCTGCAGCCGGAAGAC | |
| | | TTCGCAACTTATTACTGTCAGCAATCTTCTTCTTCTCTGATCA | |
| | | CGTTCGGACAGGGTACCAAGGTGGAGATCAAACGAACTGTG | |
| | | GCTGCACCATCTGTCTTCATCTTCCCGCCATCTGATTCACAG | |
| | | TTGAAATCTGGAACTGCCTCTGTTGTGTGCCTGCTGAATAAC | |
| | | TTCTATCCCAGAGAGGCCAAAGTACAGTGGAAGGTGGATAA | |
| | | CGCCCTCCAATCGGGTAACTCCCAGGAGAGTGTCACAGAGC | |
| | | AGGACAGCAAGGACAGCACCTACAGCCTCAGCAGCACCCTG | |
| | | ACGCTGAGCAAAGCAGACTACGAAAAACATAAAGTCTACG | |
| | | CCTGCGAAGTCACCCATCAGGGCCTGAGCTCGCCCGTCACA | |
| | | AAGAGCTTCAACAGGGGAGAGTGTTAA | |
| BOR- | Fab | GAGATCTCCGAGGTTCAGCTGGTGGAGTCTGGCGGTGGCCT | 108 |
| N-48 | Heavy | GGTGCAGCCAGGGGGCTCACTCCGTTTGTCCTGTGCAGCTTC | |
| | Chain | TGGCTTCAACTTCTCTTATTCTTCTATACACTGGGTGCGTCA | |
| | | GGCCCCGGGTAAGGGCCTGGAATGGGTTGCATCTATTTATC | |
| | | CTTATTCTGGCTCTACTTATTATGCCGATAGCGTCAAGGGCC | |
| | | GTTTCACTATAAGCGCAGACACATCCAAAAACACAGCCTAC | |
| | | CTACAAATGAACAGCTTAAGAGCTGAGGACACTGCCGTCTA | |
| | | TTATTGTGCTCGCAAACAGGTTTCTGGTTGGTGGTGGTACTA | |
| | | CGGTTTTGACTACTGGGGTCAAGGAACCCTGGTCACCGTCTC | |
| | | CTCGGCCTCCACCAAGGGTCCATCGGTCTTCCCCCTGGCACC | |
| | | CTCCTCCAAGAGCACCTCTGGGGGCACAGCGGCCCTGGGCT | |
| | | GCCTGGTCAAGGACTACTTCCCCGAACCGGTGACGGTGTCG | |
| | | TGGAACTCAGGCGCCCTGACCAGCGGCGTGCACACCTTCCC | |
| | | GGCTGTCCTACAGTCCTCAGGACTCTACTCCCTCAGCAGCGT | |
| | | GGTGACCGTGCCCTCCAGCAGCTTGGGCACCCAGACCTACA | |
| | | TCTGCAACGTGAATCACAAGCCCAGCAACACCAAGGTCGAC | |
| | | AAGAAAGTTGAGCCCAAATCTTGTGACAAAACTCACACATA | |
| | | A | |
| | Fab | TCCGATATCCAGATGACCCAGTCCCCGAGCTCCCTGTCCGCC | 109 |
| | Light | TCTGTGGGCGATAGGGTCACCATCACCTGCCGTGCCAGTCA | |
| | Chain | GTCCGTGTCCAGCGCTGTAGCCTGGTATCAACAGAAACCAG | |
| | | GAAAAGCTCCGAAGCTTCTGATTTACTCGGCATCCAGCCTCT | |
| | | ACTCTGGAGTCCCTTCTCGCTTCTCTGGTAGCCGTTCCGGGA | |
| | | CGGATTTCACTCTGACCATCAGCAGTCTGCAGCCGGAAGAC | |
| | | TTCGCAACTTATTACTGTCAGCAATCTTACTGGTGGCCGATC | |
| | | ACGTTCGGACAGGGTACCAAGGTGGAGATCAAACGAACTGT | |
| | | GGCTGCACCATCTGTCTTCATCTTCCCGCCATCTGATTCACA | |
| | | GTTGAAATCTGGAACTGCCTCTGTTGTGTGCCTGCTGAATAA | |
| | | CTTCTATCCCAGAGAGGCCAAAGTACAGTGGAAGGTGGATA | |
| | | ACGCCCTCCAATCGGGTAACTCCCAGGAGAGTGTCACAGAG | |
| | | CAGGACAGCAAGGACAGCACCTACAGCCTCAGCAGCACCCT | |
| | | GACGCTGAGCAAAGCAGACTACGAAAAACATAAAGTCTAC | |
| | | GCCTGCGAAGTCACCCATCAGGGCCTGAGCTCGCCCGTCAC | |
| | | AAAGAGCTTCAACAGGGGAGAGTGTTAA | |
| BOR- | Fab | GAGATCTCCGAGGTTCAGCTGGTGGAGTCTGGCGGTGGCCT | 110 |
| N-49 | Heavy | GGTGCAGCCAGGGGGCTCACTCCGTTTGTCCTGTGCAGCTTC | |
| | Chain | TGGCTTCAACGTCTCTTCTTATTCTATACACTGGGTGCGTCA | |
| | | GGCCCCGGGTAAGGGCCTGGAATGGGTTGCATCTATTTATT | |
| | | CTTCTTCTAGCTCTACTTATTATGCCGATAGCGTCAAGGGCC | |
| | | GTTTCACTATAAGCGCAGACACATCCAAAAACACAGCCTAC | |
| | | CTACAAATGAACAGCTTAAGAGCTGAGGACACTGCCGTCTA | |
| | | TTATTGTGCTCGCCGTTACTACCATTACCGTTACCCGCTGTT | |
| | | CTGGGGTTGGTACGGTATGGACTACTGGGGTCAAGGAACCC | |
| | | TGGTCACCGTCTCCTCGGCCTCCACCAAGGGTCCATCGGTCT | |
| | | TCCCCCTGGCACCCTCCTCCAAGAGCACCTCTGGGGGCACA | |
| | | GCGGCCCTGGGCTGCCTGGTCAAGGACTACTTCCCCGAACC | |
| | | GGTGACGGTGTCGTGGAACTCAGGCGCCCTGACCAGCGGCG | |
| | | TGCACACCTTCCCGGCTGTCCTACAGTCCTCAGGACTCTACT | |
| | | CCCTCAGCAGCGTGGTGACCGTGCCCTCCAGCAGCTTGGGC | |
| | | ACCCAGACCTACATCTGCAACGTGAATCACAAGCCCAGCAA | |
| | | CACCAAGGTCGACAAGAAAGTTGAGCCCAAATCTTGTGACA | |
| | | AAACTCACACATAA | |
| | Fab | TCCGATATCCAGATGACCCAGTCCCCGAGCTCCCTGTCCGCC | 111 |
| | Light | TCTGTGGGCGATAGGGTCACCATCACCTGCCGTGCCAGTCA | |
| | Chain | GTCCGTGTCCAGCGCTGTAGCCTGGTATCAACAGAAACCAG | |
| | | GAAAAGCTCCGAAGCTTCTGATTTACTCGGCATCCAGCCTCT | |
| | | ACTCTGGAGTCCCTTCTCGCTTCTCTGGTAGCCGTTCCGGGA | |
| | | CGGATTTCACTCTGACCATCAGCAGTCTGCAGCCGGAAGAC | |
| | | TTCGCAACTTATTACTGTCAGCAATCTTTCTGGTGGCCGATC | |
| | | ACGTTCGGACAGGGTACCAAGGTGGAGATCAAACGAACTGT | |
| | | GGCTGCACCATCTGTCTTCATCTTCCCGCCATCTGATTCACA | |
| | | GTTGAAATCTGGAACTGCCTCTGTTGTGTGCCTGCTGAATAA | |
| | | CTTCTATCCCAGAGAGGCCAAAGTACAGTGGAAGGTGGATA | |
| | | ACGCCCTCCAATCGGGTAACTCCCAGGAGAGTGTCACAGAG | |
| | | CAGGACAGCAAGGACAGCACCTACAGCCTCAGCAGCACCCT | |
| | | GACGCTGAGCAAAGCAGACTACGAAAAACATAAAGTCTAC | |
| | | GCCTGCGAAGTCACCCATCAGGGCCTGAGCTCGCCCGTCAC | |
| | | AAAGAGCTTCAACAGGGGAGAGTGTTAA | |
| BOR- | Fab | GAGATCTCCGAGGTTCAGCTGGTGGAGTCTGGCGGTGGCCT | 112 |
| N-50 | Heavy | GGTGCAGCCAGGGGGCTCACTCCGTTTGTCCTGTGCAGCTTC | |
| | Chain | TGGCTTCAACGTCTATTCTTATTCTATACACTGGGTGCGTCA | |
| | | GGCCCCGGGTAAGGGCCTGGAATGGGTTGCATCTATTTATT | |
| | | CTTATTCTGGCTATACTTATTATGCCGATAGCGTCAAGGGCC | |
| | | GTTTCACTATAAGCGCAGACACATCCAAAAACACAGCCTAC | |
| | | CTACAAATGAACAGCTTAAGAGCTGAGGACACTGCCGTCTA | |
| | | TTATTGTGCTCGCTCTACTTACCCGCATGTTTACCTGTACTTC | |
| | | GGTTTGGACTACTGGGGTCAAGGAACCCTGGTCACCGTCTC | |
| | | CTCGGCCTCCACCAAGGGTCCATCGGTCTTCCCCCTGGCACC | |
| | | CTCCTCCAAGAGCACCTCTGGGGGCACAGCGGCCCTGGGCT | |
| | | GCCTGGTCAAGGACTACTTCCCCGAACCGGTGACGGTGTCG | |
| | | TGGAACTCAGGCGCCCTGACCAGCGGCGTGCACACCTTCCC | |
| | | GGCTGTCCTACAGTCCTCAGGACTCTACTCCCTCAGCAGCGT | |
| | | GGTGACCGTGCCCTCCAGCAGCTTGGGCACCCAGACCTACA | |
| | | TCTGCAACGTGAATCACAAGCCCAGCAACACCAAGGTCGAC | |
| | | AAGAAAGTTGAGCCCAAATCTTGTGACAAAACTCACACATA | |
| | | A | |
| | Fab | TCCGATATCCAGATGACCCAGTCCCCGAGCTCCCTGTCCGCC | 113 |
| | Light | TCTGTGGGCGATAGGGTCACCATCACCTGCCGTGCCAGTCA | |
| | Chain | GTCCGTGTCCAGCGCTGTAGCCTGGTATCAACAGAAACCAG | |
| | | GAAAAGCTCCGAAGCTTCTGATTTACTCGGCATCCAGCCTCT | |
| | | ACTCTGGAGTCCCTTCTCGCTTCTCTGGTAGCCGTTCCGGGA | |
| | | CGGATTTCACTCTGACCATCAGCAGTCTGCAGCCGGAAGAC | |
| | | TTCGCAACTTATTACTGTCAGCAATCTTACTACTGGCCGGTC | |
| | | ACGTTCGGACAGGGTACCAAGGTGGAGATCAAACGAACTGT | |
| | | GGCTGCACCATCTGTCTTCATCTTCCCGCCATCTGATTCACA | |
| | | GTTGAAATCTGGAACTGCCTCTGTTGTGTGCCTGCTGAATAA | |
| | | CTTCTATCCCAGAGAGGCCAAAGTACAGTGGAAGGTGGATA | |
| | | ACGCCCTCCAATCGGGTAACTCCCAGGAGAGTGTCACAGAG | |
| | | CAGGACAGCAAGGACAGCACCTACAGCCTCAGCAGCACCCT | |
| | | GACGCTGAGCAAAGCAGACTACGAAAAACATAAAGTCTAC | |
| | | GCCTGCGAAGTCACCCATCAGGGCCTGAGCTCGCCCGTCAC | |
| | | AAAGAGCTTCAACAGGGGAGAGTGTTAA | |
| BOR- | Fab | GAGATCTCCGAGGTTCAGCTGGTGGAGTCTGGCGGTGGCCT | 114 |
| C-56 | Heavy | GGTGCAGCCAGGGGGCTCACTCCGTTTGTCCTGTGCAGCTTC | |
| | Chain | TGGCTTCAACATCTCTTCTTCTTCTATACACTGGGTGCGTCA | |
| | | GGCCCCGGGTAAGGGCCTGGAATGGGTTGCATATATTTCTT | |
| | | CTTATTCTGGCTCTACTTATTATGCCGATAGCGTCAAGGGCC | |
| | | GTTTCACTATAAGCGCAGACACATCCAAAAACACAGCCTAC | |
| | | CTACAAATGAACAGCTTAAGAGCTGAGGACACTGCCGTCTA | |
| | | TTATTGTGCTCGCTACTACCATTCTCCGCTGTGGTACTACTCT | |
| | | TACTCTGGTATGGACTACTGGGGTCAAGGAACCCTGGTCAC | |
| | | CGTCTCCTCGGCCTCCACCAAGGGTCCATCGGTCTTCCCCCT | |
| | | GGCACCCTCCTCCAAGAGCACCTCTGGGGGCACAGCGGCCC | |
| | | TGGGCTGCCTGGTCAAGGACTACTTCCCCGAACCGGTGACG | |
| | | GTGTCGTGGAACTCAGGCGCCCTGACCAGCGGCGTGCACAC | |
| | | CTTCCCGGCTGTCCTACAGTCCTCAGGACTCTACTCCCTCAG | |
| | | CAGCGTGGTGACCGTGCCCTCCAGCAGCTTGGGCACCCAGA | |
| | | CCTACATCTGCAACGTGAATCACAAGCCCAGCAACACCAAG | |
| | | GTCGACAAGAAAGTTGAGCCCAAATCTTGTGACAAAACTCA | |
| | | CACATAA | |
| | Fab | TCCGATATCCAGATGACCCAGTCCCCGAGCTCCCTGTCCGCC | 115 |
| | Light | TCTGTGGGCGATAGGGTCACCATCACCTGCCGTGCCAGTCA | |
| | Chain | GTCCGTGTCCAGCGCTGTAGCCTGGTATCAACAGAAACCAG | |
| | | GAAAAGCTCCGAAGCTTCTGATTTACTCGGCATCCAGCCTCT | |
| | | ACTCTGGAGTCCCTTCTCGCTTCTCTGGTAGCCGTTCCGGGA | |
| | | CGGATTTCACTCTGACCATCAGCAGTCTGCAGCCGGAAGAC | |
| | | TTCGCAACTTATTACTGTCAGCAAGACTACTCTTGGTCTCCG | |
| | | ATCACGTTCGGACAGGGTACCAAGGTGGAGATCAAACGAAC | |
| | | TGTGGCTGCACCATCTGTCTTCATCTTCCCGCCATCTGATTC | |
| | | ACAGTTGAAATCTGGAACTGCCTCTGTTGTGTGCCTGCTGAA | |
| | | TAACTTCTATCCCAGAGAGGCCAAAGTACAGTGGAAGGTGG | |
| | | ATAACGCCCTCCAATCGGGTAACTCCCAGGAGAGTGTCACA | |
| | | GAGCAGGACAGCAAGGACAGCACCTACAGCCTCAGCAGCA | |
| | | CCCTGACGCTGAGCAAAGCAGACTACGAAAAACATAAAGTC | |
| | | TACGCCTGCGAAGTCACCCATCAGGGCCTGAGCTCGCCCGT | |
| | | CACAAAGAGCTTCAACAGGGGAGAGTGTTAA | |
| BOR- | Fab | GAGATCTCCGAGGTTCAGCTGGTGGAGTCTGGCGGTGGCCT | 116 |
| C-57 | Heavy | GGTGCAGCCAGGGGGCTCACTCCGTTTGTCCTGTGCAGCTTC | |
| | Chain | TGGCTTCAACATCTCTTCTTCTTCTATACACTGGGTGCGTCA | |
| | | GGCCCCGGGTAAGGGCCTGGAATGGGTTGCATCTATTTCTTC | |
| | | TTATTATAGCTATACTTATTATGCCGATAGCGTCAAGGGCCG | |
| | | TTTCACTATAAGCGCAGACACATCCAAAAACACAGCCTACC | |
| | | TACAAATGAACAGCTTAAGAGCTGAGGACACTGCCGTCTAT | |
| | | TATTGTGCTCGCGGTGCTTGGTACGGTTACCTGGGTTACTAC | |
| | | GCTTACTACTACTACGCTTTGGACTACTGGGGTCAAGGAAC | |
| | | CCTGGTCACCGTCTCCTCGGCCTCCACCAAGGGTCCATCGGT | |
| | | CTTCCCCCTGGCACCCTCCTCCAAGAGCACCTCTGGGGGCAC | |
| | | AGCGGCCCTGGGCTGCCTGGTCAAGGACTACTTCCCCGAAC | |
| | | CGGTGACGGTGTCGTGGAACTCAGGCGCCCTGACCAGCGGC | |
| | | GTGCACACCTTCCCGGCTGTCCTACAGTCCTCAGGACTCTAC | |
| | | TCCCTCAGCAGCGTGGTGACCGTGCCCTCCAGCAGCTTGGG | |
| | | CACCCAGACCTACATCTGCAACGTGAATCACAAGCCCAGCA | |
| | | ACACCAAGGTCGACAAGAAAGTTGAGCCCAAATCTTGTGAC | |
| | | AAAACTCACACATAA | |
| | Fab | TCCGATATCCAGATGACCCAGTCCCCGAGCTCCCTGTCCGCC | 117 |
| | Light | TCTGTGGGCGATAGGGTCACCATCACCTGCCGTGCCAGTCA | |
| | Chain | GTCCGTGTCCAGCGCTGTAGCCTGGTATCAACAGAAACCAG | |
| | | GAAAAGCTCCGAAGCTTCTGATTTACTCGGCATCCAGCCTCT | |
| | | ACTCTGGAGTCCCTTCTCGCTTCTCTGGTAGCCGTTCCGGGA | |
| | | CGGATTTCACTCTGACCATCAGCAGTCTGCAGCCGGAAGAC | |
| | | TTCGCAACTTATTACTGTCAGCAAATGGGTTACGAAACTCTG | |
| | | ATCACGTTCGGACAGGGTACCAAGGTGGAGATCAAACGAAC | |
| | | TGTGGCTGCACCATCTGTCTTCATCTTCCCGCCATCTGATTC | |
| | | ACAGTTGAAATCTGGAACTGCCTCTGTTGTGTGCCTGCTGAA | |
| | | TAACTTCTATCCCAGAGAGGCCAAAGTACAGTGGAAGGTGG | |
| | | ATAACGCCCTCCAATCGGGTAACTCCCAGGAGAGTGTCACA | |
| | | GAGCAGGACAGCAAGGACAGCACCTACAGCCTCAGCAGCA | |
| | | CCCTGACGCTGAGCAAAGCAGACTACGAAAAACATAAAGTC | |
| | | TACGCCTGCGAAGTCACCCATCAGGGCCTGAGCTCGCCCGT | |
| | | CACAAAGAGCTTCAACAGGGGAGAGTGTTAA | |
| BOR- | Fab | GAGATCTCCGAGGTTCAGCTGGTGGAGTCTGGCGGTGGCCT | 118 |
| N-65 | Heavy | GGTGCAGCCAGGGGGCTCACTCCGTTTGTCCTGTGCAGCTTC | |
| | Chain | TGGCTTCAACTTTTCTTCTTCTTCTATACACTGGGTGCGTCAG | |
| | | GCCCCGGGTAAGGGCCTGGAATGGGTTGCATCTATTTCTTCT | |
| | | TCTTCTGGCTCTACTTCTTATGCCGATAGCGTCAAGGGCCGT | |
| | | TTCACTATAAGCGCAGACACATCCAAAAACACAGCCTACCT | |
| | | ACAAATGAACAGCTTAAGAGCTGAGGACACTGCCGTCTATT | |
| | | ATTGTGCTCGCGAATCTTCTCAGACTAAAAAATACTGGTACT | |
| | | GGGTTGTTCAGTGGTACACTGCTATGGACTACTGGGGTCAA | |
| | | GGAACCCTGGTCACCGTCTCCTCGGCCTCCACCAAGGGTCC | |
| | | ATCGGTCTTCCCCCTGGCACCCTCCTCCAAGAGCACCTCTGG | |
| | | GGGCACAGCGGCCCTGGGCTGCCTGGTCAAGGACTACTTCC | |
| | | CCGAACCGGTGACGGTGTCGTGGAACTCAGGCGCCCTGACC | |
| | | AGCGGCGTGCACACCTTCCCGGCTGTCCTACAGTCCTCAGG | |
| | | ACTCTACTCCCTCAGCAGCGTGGTGACCGTGCCCTCCAGCA | |
| | | GCTTGGGCACCCAGACCTACATCTGCAACGTGAATCACAAG | |
| | | CCCAGCAACACCAAGGTCGACAAGAAAGTTGAGCCCAAATC | |
| | | TTGTGACAAAACTCACACATAA | |
| | Fab | TCCGATATCCAGATGACCCAGTCCCCGAGCTCCCTGTCCGCC | 119 |
| | Light | TCTGTGGGCGATAGGGTCACCATCACCTGCCGTGCCAGTCA | |
| | Chain | GTCCGTGTCCAGCGCTGTAGCCTGGTATCAACAGAAACCAG | |
| | | GAAAAGCTCCGAAGCTTCTGATTTACTCGGCATCCAGCCTCT | |
| | | ACTCTGGAGTCCCTTCTCGCTTCTCTGGTAGCCGTTCCGGGA | |
| | | CGGATTTCACTCTGACCATCAGCAGTCTGCAGCCGGAAGAC | |
| | | TTCGCAACTTATTACTGTCAGCAATACTCTCAGTGGCTGATC | |
| | | ACGTTCGGACAGGGTACCAAGGTGGAGATCAAACGAACTGT | |
| | | GGCTGCACCATCTGTCTTCATCTTCCCGCCATCTGATTCACA | |
| | | GTTGAAATCTGGAACTGCCTCTGTTGTGTGCCTGCTGAATAA | |
| | | CTTCTATCCCAGAGAGGCCAAAGTACAGTGGAAGGTGGATA | |
| | | ACGCCCTCCAATCGGGTAACTCCCAGGAGAGTGTCACAGAG | |
| | | CAGGACAGCAAGGACAGCACCTACAGCCTCAGCAGCACCCT | |
| | | GACGCTGAGCAAAGCAGACTACGAAAAACATAAAGTCTAC | |
| | | GCCTGCGAAGTCACCCATCAGGGCCTGAGCTCGCCCGTCAC | |
| | | AAAGAGCTTCAACAGGGGAGAGTGTTAA | |
| BOR- | Fab | GAGATCTCCGAGGTTCAGCTGGTGGAGTCTGGCGGTGGCCT | 120 |
| C-58 | Heavy | GGTGCAGCCAGGGGGCTCACTCCGTTTGTCCTGTGCAGCTTC | |
| | Chain | TGGCTTCAACGTCTCTTCTTATTCTATACACTGGGTGCGTCA | |
| | | GGCCCCGGGTAAGGGCCTGGAATGGGTTGCATCTATTTCTC | |
| | | CTTATTATGGCTATACTTCTTATGCCGATAGCGTCAAGGGCC | |
| | | GTTTCACTATAAGCGCAGACACATCCAAAAACACAGCCTAC | |
| | | CTACAAATGAACAGCTTAAGAGCTGAGGACACTGCCGTCTA | |
| | | TTATTGTGCTCGCCATTACTACTACGGTTTCTCTTGGTTCTCT | |
| | | TCTGGTTACGCTATGGACTACTGGGGTCAAGGAACCCTGGT | |
| | | CACCGTCTCCTCGGCCTCCACCAAGGGTCCATCGGTCTTCCC | |
| | | CCTGGCACCCTCCTCCAAGAGCACCTCTGGGGGCACAGCGG | |
| | | CCCTGGGCTGCCTGGTCAAGGACTACTTCCCCGAACCGGTG | |
| | | ACGGTGTCGTGGAACTCAGGCGCCCTGACCAGCGGCGTGCA | |
| | | CACCTTCCCGGCTGTCCTACAGTCCTCAGGACTCTACTCCCT | |
| | | CAGCAGCGTGGTGACCGTGCCCTCCAGCAGCTTGGGCACCC | |
| | | AGACCTACATCTGCAACGTGAATCACAAGCCCAGCAACACC | |
| | | AAGGTCGACAAGAAAGTTGAGCCCAAATCTTGTGACAAAAC | |
| | | TCACACATAA | |
| | Fab | TCCGATATCCAGATGACCCAGTCCCCGAGCTCCCTGTCCGCC | 121 |
| | Light | TCTGTGGGCGATAGGGTCACCATCACCTGCCGTGCCAGTCA | |
| | Chain | GTCCGTGTCCAGCGCTGTAGCCTGGTATCAACAGAAACCAG | |
| | | GAAAAGCTCCGAAGCTTCTGATTTACTCGGCATCCAGCCTCT | |
| | | ACTCTGGAGTCCCTTCTCGCTTCTCTGGTAGCCGTTCCGGGA | |
| | | CGGATTTCACTCTGACCATCAGCAGTCTGCAGCCGGAAGAC | |
| | | TTCGCAACTTATTACTGTCAGCAAGGTTCTTCTTCTCCGATC | |
| | | ACGTTCGGACAGGGTACCAAGGTGGAGATCAAACGAACTGT | |
| | | GGCTGCACCATCTGTCTTCATCTTCCCGCCATCTGATTCACA | |
| | | GTTGAAATCTGGAACTGCCTCTGTTGTGTGCCTGCTGAATAA | |
| | | CTTCTATCCCAGAGAGGCCAAAGTACAGTGGAAGGTGGATA | |
| | | ACGCCCTCCAATCGGGTAACTCCCAGGAGAGTGTCACAGAG | |
| | | CAGGACAGCAAGGACAGCACCTACAGCCTCAGCAGCACCCT | |
| | | GACGCTGAGCAAAGCAGACTACGAAAAACATAAAGTCTAC | |
| | | GCCTGCGAAGTCACCCATCAGGGCCTGAGCTCGCCCGTCAC | |
| | | AAAGAGCTTCAACAGGGGAGAGTGTTAA | |

TABLE 3
Summary of SEQ ID NOs. (Amino Acid Sequences)

| | HCD | HCD | HCD | LCD | LCD | LCD | | | HFR | HFR | HFR | HFR | LFR | LFR | LFR | LFR |
|---|---|---|---|---|---|---|---|---|---|---|---|---|---|---|---|---|
| Clone | R1 | R2 | R3 | R1 | R2 | R3 | VH | VL | W1 | W2 | W3 | W4 | W1 | W2 | W3 | W4 |
| BOR-N-54 | 1 | 2 | 3 | 4 | 5 | 6 | 7 | 8 | 9 | 10 | 11 | 12 | 13 | 14 | 15 | 16 |
| BOR-C-59 | 17 | 18 | 19 | 4 | 5 | 20 | 21 | 22 | 9 | 10 | 11 | 12 | 13 | 14 | 15 | 16 |
| BOR-C-60 | 23 | 24 | 25 | 4 | 5 | 26 | 27 | 28 | 9 | 10 | 11 | 12 | 13 | 14 | 15 | 16 |
| BOR-N-51 | 29 | 30 | 31 | 4 | 5 | 32 | 33 | 34 | 9 | 10 | 11 | 12 | 13 | 14 | 15 | 16 |
| BOR-N-55 | 35 | 36 | 37 | 4 | 5 | 38 | 39 | 40 | 9 | 10 | 11 | 12 | 13 | 14 | 15 | 16 |
| BOR-C-62 | 41 | 42 | 43 | 4 | 5 | 38 | 44 | 40 | 9 | 10 | 11 | 12 | 13 | 14 | 15 | 16 |
| BOR-C-61 | 45 | 42 | 46 | 4 | 5 | 38 | 47 | 40 | 9 | 10 | 11 | 12 | 13 | 14 | 15 | 16 |
| BOR-C-64 | 35 | 48 | 49 | 4 | 5 | 50 | 51 | 52 | 9 | 10 | 11 | 12 | 13 | 14 | 15 | 16 |
| BOR-C-63 | 53 | 54 | 55 | 4 | 5 | 38 | 56 | 40 | 9 | 10 | 11 | 12 | 13 | 14 | 15 | 16 |
| BOR-N-48 | 57 | 58 | 59 | 4 | 5 | 60 | 61 | 62 | 9 | 10 | 11 | 12 | 13 | 14 | 15 | 16 |
| BOR-N-49 | 41 | 63 | 64 | 4 | 5 | 65 | 66 | 67 | 9 | 10 | 11 | 12 | 13 | 14 | 15 | 16 |
| BOR-N-50 | 29 | 68 | 69 | 4 | 5 | 70 | 71 | 72 | 9 | 10 | 11 | 12 | 13 | 14 | 15 | 16 |
| BOR-C-56 | 73 | 74 | 75 | 4 | 5 | 76 | 77 | 78 | 9 | 10 | 11 | 12 | 13 | 14 | 15 | 16 |
| BOR-C-57 | 73 | 79 | 80 | 4 | 5 | 81 | 82 | 83 | 9 | 10 | 11 | 12 | 13 | 14 | 15 | 16 |
| BOR-N-65 | 53 | 42 | 84 | 4 | 5 | 85 | 86 | 87 | 9 | 10 | 11 | 12 | 13 | 14 | 15 | 16 |
| BOR-C-58 | 41 | 88 | 89 | 4 | 5 | 90 | 91 | 92 | 9 | 10 | 11 | 12 | 13 | 14 | 15 | 16 |

TABLE 4
Fab Heavy and Light Chains

| | | | SEQ |
|---|---|---|---|
| | | | ID |
| Clone | Region | Sequence | NO: |
| BOR- | Fab | EISEVQLVESGGGLVQPGGSLRLSCAASGFNIYYSSIHWVRQAP | 124 |
| N-54 | Heavy | GKGLEWVASIYPYYGSTSYADSVKGRFTISADTSKNTAYLQMN | |
| | Chain | SLRAEDTAVYYCARKWYYGDVGMDYWGQGTLVTVSSASTKG | |
| | | PSVFPLAPSSKSTSGGTAALGCLVKDYFPEPVTVSWNSGALTSG | |
| | | VHTFPAVLQSSGLYSLSSVVTVPSSSLGTQTYICNVNHKPSNTK | |
| | | VDKKVEPKSCDKTHT | |
| | Fab | SDIQMTQSPSSLSASVGDRVTITCRASQSVSSAVAWYQQKPGKA | 125 |
| | Light | PKLLIYSASSLYSGVPSRFSGSRSGTDFTLTISSLQPEDFATYYCQ | |
| | Chain | QSEYRLITFGQGTKVEIKRTVAAPSVFIFPPSDSQLKSGTASVVC | |
| | | LLNNFYPREAKVQWKVDNALQSGNSQESVTEQDSKDSTYSLSS | |
| | | TLTLSKADYEKHKVYACEVTHQGLSSPVTKSFNRGEC | |
| BOR- | Fab | EISEVQLVESGGGLVQPGGSLRLSCAASGFNLSSSYIHWVRQAP | 126 |
| C-59 | Heavy | GKGLEWVAYIYSSSGSTYYADSVKGRFTISADTSKNTAYLQMN | |
| | Chain | SLRAEDTAVYYCARWPTYYYWLVGWMYDVGAFDYWGQGTL | |
| | | VTVSSASTKGPSVFPLAPSSKSTSGGTAALGCLVKDYFPEPVTVS | |
| | | WNSGALTSGVHTFPAVLQSSGLYSLSSVVTVPSSSLGTQTYICN | |
| | | VNHKPSNTKVDKKVEPKSCDKTHT | |
| | Fab | SDIQMTQSPSSLSASVGDRVTITCRASQSVSSAVAWYQQKPGKA | 127 |
| | Light | PKLLIYSASSLYSGVPSRFSGSRSGTDFTLTISSLQPEDFATYYCQ | |
| | Chain | QSSSEYPVTFGQGTKVEIKRTVAAPSVFIFPPSDSQLKSGTASVV | |
| | | CLLNNFYPREAKVQWKVDNALQSGNSQESVTEQDSKDSTYSLS | |
| | | STLTLSKADYEKHKVYACEVTHQGLSSPVTKSFNRGEC | |
| BOR- | Fab | EISEVQLVESGGGLVQPGGSLRLSCAASGFNFSYSYIHWVRQAP | 128 |
| C-60 | Heavy | GKGLEWVAYISSSSGYTYYADSVKGRFTISADTSKNTAYLQMN | |
| | Chain | SLRAEDTAVYYCARSGLYSHGVWQNWYGYMGFDYWGQGTL | |
| | | VTVSSASTKGPSVFPLAPSSKSTSGGTAALGCLVKDYFPEPVTVS | |
| | | WNSGALTSGVHTFPAVLQSSGLYSLSSVVTVPSSSLGTQTYICN | |
| | | VNHKPSNTKVDKKVEPKSCDKTHT | |
| | Fab | SDIQMTQSPSSLSASVGDRVTITCRASQSVSSAVAWYQQKPGKA | 129 |
| | Light | PKLLIYSASSLYSGVPSRFSGSRSGTDFTLTISSLQPEDFATYYCQ | |
| | Chain | QSLQWPITFGQGTKVEIKRTVAAPSVFIFPPSDSQLKSGTASVVC | |
| | | LLNNFYPREAKVQWKVDNALQSGNSQESVTEQDSKDSTYSLSS | |
| | | TLTLSKADYEKHKVYACEVTHQGLSSPVTKSFNRGEC | |
| BOR- | Fab | EISEVQLVESGGGLVQPGGSLRLSCAASGFNVYSYSIHWVRQAP | 130 |
| N-51 | Heavy | GKGLEWVASIYSSYSYTSYADSVKGRFTISADTSKNTAYLQMN | |
| | Chain | SLRAEDTAVYYCARKYHFKPTYWGAMDYWGQGTLVTVSSAS | |
| | | TKGPSVFPLAPSSKSTSGGTAALGCLVKDYFPEPVTVSWNSGAL | |
| | | TSGVHTFPAVLQSSGLYSLSSVVTVPSSSLGTQTYICNVNHKPSN | |
| | | TKVDKKVEPKSCDKTHT | |
| | Fab | SDIQMTQSPSSLSASVGDRVTITCRASQSVSSAVAWYQQKPGKA | 131 |
| | Light | PKLLIYSASSLYSGVPSRFSGSRSGTDFTLTISSLQPEDFATYYCQ | |
| | Chain | QSSLYYPLTFGQGTKVEIKRTVAAPSVFIFPPSDSQLKSGTASVV | |
| | | CLLNNFYPREAKVQWKVDNALQSGNSQESVTEQDSKDSTYSLS | |
| | | STLTLSKADYEKHKVYACEVTHQGLSSPVTKSFNRGEC | |
| BOR- | Fab | EISEVQLVESGGGLVQPGGSLRLSCAASGFNVYYSSIHWVRQAP | 132 |
| N-55 | Heavy | GKGLEWVASISPYYGSTSYADSVKGRFTISADTSKNTAYLQMN | |
| | Chain | SLRAEDTAVYYCARRWYYTKVFMWGMDYWGQGTLVTVSSAS | |
| | | TKGPSVFPLAPSSKSTSGGTAALGCLVKDYFPEPVTVSWNSGAL | |
| | | TSGVHTFPAVLQSSGLYSLSSVVTVPSSSLGTQTYICNVNHKPSN | |
| | | TKVDKKVEPKSCDKTHT | |
| | Fab | SDIQMTQSPSSLSASVGDRVTITCRASQSVSSAVAWYQQKPGKA | 133 |
| | Light | PKLLIYSASSLYSGVPSRFSGSRSGTDFTLTISSLQPEDFATYYCQ | |
| | Chain | QSSSSLITFGQGTKVEIKRTVAAPSVFIFPPSDSQLKSGTASVVCL | |
| | | LNNFYPREAKVQWKVDNALQSGNSQESVTEQDSKDSTYSLSST | |
| | | LTLSKADYEKHKVYACEVTHQGLSSPVTKSFNRGEC | |
| BOR- | Fab | EISEVQLVESGGGLVQPGGSLRLSCAASGFNVSSYSIHWVRQAP | 134 |
| C-62 | Heavy | GKGLEWVASISSSSGSTSYADSVKGRFTISADTSKNTAYLQMNS | |
| | Chain | LRAEDTAVYYCARESNQYYYYYNVLWWYQAFDYWGQGTLV | |
| | | TVSSASTKGPSVFPLAPSSKSTSGGTAALGCLVKDYFPEPVTVS | |
| | | WNSGALTSGVHTFPAVLQSSGLYSLSSVVTVPSSSLGTQTYICN | |
| | | VNHKPSNTKVDKKVEPKSCDKTHT | |
| | Fab | SDIQMTQSPSSLSASVGDRVTITCRASQSVSSAVAWYQQKPGKA | 133 |
| | Light | PKLLIYSASSLYSGVPSRFSGSRSGTDFTLTISSLQPEDFATYYCQ | |
| | Chain | QSSSSLITFGQGTKVEIKRTVAAPSVFIFPPSDSQLKSGTASVVCL | |
| | | LNNFYPREAKVQWKVDNALQSGNSQESVTEQDSKDSTYSLSST | |
| | | LTLSKADYEKHKVYACEVTHQGLSSPVTKSFNRGEC | |
| BOR- | Fab | EISEVQLVESGGGLVQPGGSLRLSCAASGFNFYSSYIHWVRQAP | 135 |
| C-61 | Heavy | GKGLEWVASISSSSGSTSYADSVKGRFTISADTSKNTAYLQMNS | |
| | Chain | LRAEDTAVYYCARRSGGGLDYWGQGTLVTVSSASTKGPSVFPL | |
| | | APSSKSTSGGTAALGCLVKDYFPEPVTVSWNSGALTSGVHTFPA | |
| | | VLQSSGLYSLSSVVTVPSSSLGTQTYICNVNHKPSNTKVDKKVE | |
| | | PKSCDKTHT | |
| | Fab | SDIQMTQSPSSLSASVGDRVTITCRASQSVSSAVAWYQQKPGKA | 133 |
| | Light | PKLLIYSASSLYSGVPSRFSGSRSGTDFTLTISSLQPEDFATYYCQ | |
| | Chain | QSSSSLITFGQGTKVEIKRTVAAPSVFIFPPSDSQLKSGTASVVCL | |
| | | LNNFYPREAKVQWKVDNALQSGNSQESVTEQDSKDSTYSLSST | |
| | | LTLSKADYEKHKVYACEVTHQGLSSPVTKSFNRGEC | |
| BOR- | Fab | EISEVQLVESGGGLVQPGGSLRLSCAASGFNVYYSSIHWVRQAP | 136 |
| C-64 | Heavy | GKGLEWVASISSYYGSTYYADSVKGRFTISADTSKNTAYLQMN | |
| | Chain | SLRAEDTAVYYCARWKWVAMDYWGQGTLVTVSSASTKGPSV | |
| | | FPLAPSSKSTSGGTAALGCLVKDYFPEPVTVSWNSGALTSGVHT | |
| | | FPAVLQSSGLYSLSSVVTVPSSSLGTQTYICNVNHKPSNTKVDK | |
| | | KVEPKSCDKTHT | |
| | Fab | SDIQMTQSPSSLSASVGDRVTITCRASQSVSSAVAWYQQKPGKA | 137 |
| | Light | PKLLIYSASSLYSGVPSRFSGSRSGTDFTLTISSLQPEDFATYYCQ | |
| | Chain | QDLFMPITFGQGTKVEIKRTVAAPSVFIFPPSDSQLKSGTASVVC | |
| | | LLNNFYPREAKVQWKVDNALQSGNSQESVTEQDSKDSTYSLSS | |
| | | TLTLSKADYEKHKVYACEVTHQGLSSPVTKSFNRGEC | |
| BOR- | Fab | EISEVQLVESGGGLVQPGGSLRLSCAASGFNFSSSSIHWVRQAPG | 138 |
| C-63 | Heavy | KGLEWVASIYSYSGSTYYADSVKGRFTISADTSKNTAYLQMNS | |
| | Chain | LRAEDTAVYYCARGLYYYLPSYYIYYRSYKFGMDYWGQGTLV | |
| | | TVSSASTKGPSVFPLAPSSKSTSGGTAALGCLVKDYFPEPVTVS | |
| | | WNSGALTSGVHTFPAVLQSSGLYSLSSVVTVPSSSLGTQTYICN | |
| | | VNHKPSNTKVDKKVEPKSCDKTHT | |
| | Fab | SDIQMTQSPSSLSASVGDRVTITCRASQSVSSAVAWYQQKPGKA | 133 |
| | Light | PKLLIYSASSLYSGVPSRFSGSRSGTDFTLTISSLQPEDFATYYCQ | |
| | Chain | QSSSSLITFGQGTKVEIKRTVAAPSVFIFPPSDSQLKSGTASVVCL | |
| | | LNNFYPREAKVQWKVDNALQSGNSQESVTEQDSKDSTYSLSST | |
| | | LTLSKADYEKHKVYACEVTHQGLSSPVTKSFNRGEC | |
| BOR- | Fab | EISEVQLVESGGGLVQPGGSLRLSCAASGFNFSYSSIHWVRQAP | 139 |
| N-48 | Heavy | GKGLEWVASIYPYSGSTYYADSVKGRFTISADTSKNTAYLQMN | |
| | Chain | SLRAEDTAVYYCARKQVSGWWWYYGFDYWGQGTLVTVSSAS | |
| | | TKGPSVFPLAPSSKSTSGGTAALGCLVKDYFPEPVTVSWNSGAL | |
| | | TSGVHTFPAVLQSSGLYSLSSVVTVPSSSLGTQTYICNVNHKPSN | |
| | | TKVDKKVEPKSCDKTHT | |
| | Fab | SDIQMTQSPSSLSASVGDRVTITCRASQSVSSAVAWYQQKPGKA | 140 |
| | Light | PKLLIYSASSLYSGVPSRFSGSRSGTDFTLTISSLQPEDFATYYCQ | |
| | Chain | QSYWWPITFGQGTKVEIKRTVAAPSVFIFPPSDSQLKSGTASVVC | |
| | | LLNNFYPREAKVQWKVDNALQSGNSQESVTEQDSKDSTYSLSS | |
| | | TLTLSKADYEKHKVYACEVTHQGLSSPVTKSFNRGEC | |
| BOR- | Fab | EISEVQLVESGGGLVQPGGSLRLSCAASGFNVSSYSIHWVRQAP | 141 |
| N-49 | Heavy | GKGLEWVASIYSSSSSTYYADSVKGRFTISADTSKNTAYLQMNS | |
| | Chain | LRAEDTAVYYCARRYYHYRYPLFWGWYGMDYWGQGTLVTV | |
| | | SSASTKGPSVFPLAPSSKSTSGGTAALGCLVKDYFPEPVTVSWN | |
| | | SGALTSGVHTFPAVLQSSGLYSLSSVVTVPSSSLGTQTYICNVNH | |
| | | KPSNTKVDKKVEPKSCDKTHT | |
| | Fab | SDIQMTQSPSSLSASVGDRVTITCRASQSVSSAVAWYQQKPGKA | 142 |
| | Light | PKLLIYSASSLYSGVPSRFSGSRSGTDFTLTISSLQPEDFATYYCQ | |
| | Chain | QSFWWPITFGQGTKVEIKRTVAAPSVFIFPPSDSQLKSGTASVVC | |
| | | LLNNFYPREAKVQWKVDNALQSGNSQESVTEQDSKDSTYSLSS | |
| | | TLTLSKADYEKHKVYACEVTHQGLSSPVTKSFNRGEC | |
| BOR- | Fab | EISEVQLVESGGGLVQPGGSLRLSCAASGFNVYSYSIHWVRQAP | 143 |
| N-50 | Heavy | GKGLEWVASIYSYSGYTYYADSVKGRFTISADTSKNTAYLQMN | |
| | Chain | SLRAEDTAVYYCARSTYPHVYLYFGLDYWGQGTLVTVSSASTK | |
| | | GPSVFPLAPSSKSTSGGTAALGCLVKDYFPEPVTVSWNSGALTS | |
| | | GVHTFPAVLQSSGLYSLSSVVTVPSSSLGTQTYICNVNHKPSNT | |
| | | KVDKKVEPKSCDKTHT | |
| | Fab | SDIQMTQSPSSLSASVGDRVTITCRASQSVSSAVAWYQQKPGKA | 144 |
| | Light | PKLLIYSASSLYSGVPSRFSGSRSGTDFTLTISSLQPEDFATYYCQ | |
| | Chain | QSYYWPVTFGQGTKVEIKRTVAAPSVFIFPPSDSQLKSGTASVV | |
| | | CLLNNFYPREAKVQWKVDNALQSGNSQESVTEQDSKDSTYSLS | |
| | | STLTLSKADYEKHKVYACEVTHQGLSSPVTKSFNRGEC | |
| BOR- | Fab | EISEVQLVESGGGLVQPGGSLRLSCAASGFNISSSSIHWVRQAPG | 145 |
| C-56 | Heavy | KGLEWVAYISSYSGSTYYADSVKGRFTISADTSKNTAYLQMNS | |
| | Chain | LRAEDTAVYYCARYYHSPLWYYSYSGMDYWGQGTLVTVSSAS | |
| | | TKGPSVFPLAPSSKSTSGGTAALGCLVKDYFPEPVTVSWNSGAL | |
| | | TSGVHTFPAVLQSSGLYSLSSVVTVPSSSLGTQTYICNVNHKPSN | |
| | | TKVDKKVEPKSCDKTHT | |
| | Fab | SDIQMTQSPSSLSASVGDRVTITCRASQSVSSAVAWYQQKPGKA | 146 |
| | Light | PKLLIYSASSLYSGVPSRFSGSRSGTDFTLTISSLQPEDFATYYCQ | |
| | Chain | QDYSWSPITFGQGTKVEIKRTVAAPSVFIFPPSDSQLKSGTASVV | |
| | | CLLNNFYPREAKVQWKVDNALQSGNSQESVTEQDSKDSTYSLS | |
| | | STLTLSKADYEKHKVYACEVTHQGLSSPVTKSFNRGEC | |
| BOR- | Fab | EISEVQLVESGGGLVQPGGSLRLSCAASGFNISSSSIHWVRQAPG | 147 |
| C-57 | Heavy | KGLEWVASISSYYSYTYYADSVKGRFTISADTSKNTAYLQMNS | |
| | Chain | LRAEDTAVYYCARGAWYGYLGYYAYYYYALDYWGQGTLVT | |
| | | VSSASTKGPSVFPLAPSSKSTSGGTAALGCLVKDYFPEPVTVSW | |
| | | NSGALTSGVHTFPAVLQSSGLYSLSSVVTVPSSSLGTQTYICNVN | |
| | | HKPSNTKVDKKVEPKSCDKTHT | |
| | Fab | SDIQMTQSPSSLSASVGDRVTITCRASQSVSSAVAWYQQKPGKA | 148 |
| | Light | PKLLIYSASSLYSGVPSRFSGSRSGTDFTLTISSLQPEDFATYYCQ | |
| | Chain | QMGYETLITFGQGTKVEIKRTVAAPSVFIFPPSDSQLKSGTASVV | |
| | | CLLNNFYPREAKVQWKVDNALQSGNSQESVTEQDSKDSTYSLS | |
| | | STLTLSKADYEKHKVYACEVTHQGLSSPVTKSFNRGEC | |
| BOR- | Fab | EISEVQLVESGGGLVQPGGSLRLSCAASGFNFSSSSIHWVRQAPG | 149 |
| N-65 | Heavy | KGLEWVASISSSSGSTSYADSVKGRFTISADTSKNTAYLQMNSL | |
| | Chain | RAEDTAVYYCARESSQTKKYWYWVVQWYTAMDYWGQGTLV | |
| | | TVSSASTKGPSVFPLAPSSKSTSGGTAALGCLVKDYFPEPVTVS | |
| | | WNSGALTSGVHTFPAVLQSSGLYSLSSVVTVPSSSLGTQTYICN | |
| | | VNHKPSNTKVDKKVEPKSCDKTHT | |
| | Fab | SDIQMTQSPSSLSASVGDRVTITCRASQSVSSAVAWYQQKPGKA | 150 |
| | Light | PKLLIYSASSLYSGVPSRFSGSRSGTDFTLTISSLQPEDFATYYCQ | |
| | Chain | QYSQWLITFGQGTKVEIKRTVAAPSVFIFPPSDSQLKSGTASVVC | |
| | | LLNNFYPREAKVQWKVDNALQSGNSQESVTEQDSKDSTYSLSS | |
| | | TLTLSKADYEKHKVYACEVTHQGLSSPVTKSFNRGEC | |
| BOR- | Fab Heavy | EISEVQLVESGGGLVQPGGSLRLSCAASGFNVSSYSIHWVRQAP | 151 |
| C-58 | Chain | GKGLEWVASISPYYGYTSYADSVKGRFTISADTSKNTAYLQMN | |
| | | SLRAEDTAVYYCARHYYYGFSWFSSGYAMDYWGQGTLVTVSS | |
| | | ASTKGPSVFPLAPSSKSTSGGTAALGCLVKDYFPEPVTVSWNSG | |
| | | ALTSGVHTFPAVLQSSGLYSLSSVVTVPSSSLGTQTYICNVNHKP | |
| | | SNTKVDKKVEPKSCDKTHT | |
| | Fab | SDIQMTQSPSSLSASVGDRVTITCRASQSVSSAVAWYQQKPGKA | 152 |
| | Light | PKLLIYSASSLYSGVPSRFSGSRSGTDFTLTISSLQPEDFATYYCQ | |
| | Chain | QGSSSPITFGQGTKVEIKRTVAAPSVFIFPPSDSQLKSGTASVVCL | |
| | | LNNFYPREAKVQWKVDNALQSGNSQESVTEQDSKDSTYSLSST | |
| | | LTLSKADYEKHKVYACEVTHQGLSSPVTKSFNRGEC | |

### 1. Variant Polypeptides

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

### 2. Considerations for Substitutions

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further embodiments, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain embodiments, the substitution of amino acids whose hydropathy indices are within ±2 is included. In some aspects of the invention, those that are within ±1 are included, and in other aspects of the invention, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain embodiments, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain embodiments, the substitution of amino acids whose hydrophilicity values are within ±2 are included, in other embodiments, those which are within ±1 are included, and in still other embodiments, those within 0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein_structure.

In some embodiments of the invention, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain embodiments, conservative amino acid substitutions) may be made in the naturally occurring sequence. Substitutions can be made in that portion of the antibody that lies outside the domain(s) forming intermolecular contacts. In such embodiments, conservative amino acid substitutions can be used that do not substantially change the structural characteristics of the protein or polypeptide (e.g., one or more replacement amino acids that do not disrupt the secondary structure that characterizes the native antibody).

### VI. NUCLEIC ACIDS

In certain embodiments, nucleic acid sequences can exist in a variety of instances such as: isolated segments and recombinant vectors of incorporated sequences or recombinant polynucleotides encoding peptides and polypeptides of the disclosure, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain embodiments, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### A. Hybridization

The nucleic acids that hybridize to other nucleic acids under particular hybridization conditions. Methods for hybridizing nucleic acids are well known in the art. See, e.g., Current Protocols in Molecular Biology, John Wiley and Sons, N.Y. (1989), 6.3.1-6.3.6. As defined herein, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C. in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequence that are at least 65%, at least 70%, at least 75%, at least 80%, at least 85%, at least 90%, at least 95%, at least 96%, at least 97%, at least 98% or at least 99% identical to each other typically remain hybridized to each other.

The parameters affecting the choice of hybridization conditions and guidance for devising suitable conditions are set forth by, for example, Sambrook, Fritsch, and Maniatis (Molecular Cloning: A Laboratory Manual, Cold Spring Harbor Laboratory Press, Cold Spring Harbor, N.Y., chapters 9 and 11 (1989); Current Protocols in Molecular Biology, Ausubel et al., eds., John Wiley and Sons, Inc., sections 2.10 and 6.3-6.4 (1995), both of which are herein incorporated by reference in their entirety for all purposes) and can be readily determined by those having ordinary skill in the art based on, for example, the length and/or base composition of the DNA.

### B. Mutation

Changes can be introduced by mutation into a nucleic acid, thereby leading to changes in the amino acid sequence of a polypeptide (e.g., an antigenic peptide or polypeptide) that it encodes. Mutations can be introduced using any technique known in the art. In one embodiment, one or more particular amino acid residues are changed using, for example, a site-directed mutagenesis protocol. In another embodiment, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, eg., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

### C. Probes

In another aspect, nucleic acid molecules are suitable for use as primers or hybridization probes for the detection of nucleic acid sequences. A nucleic acid molecule can comprise only a portion of a nucleic acid sequence encoding a full-length polypeptide, for example, a fragment that can be used as a probe or primer or a fragment encoding an active portion of a given polypeptide.

In another embodiment, the nucleic acid molecules may be used as probes or PCR primers for specific nucleic acid sequences. For instance, a nucleic acid molecule probe may be used in diagnostic methods or a nucleic acid molecule PCR primer may be used to amplify regions of DNA that could be used, inter alia, to isolate nucleic acid sequences for use in producing the engineered cells of the disclosure. In a preferred embodiment, the nucleic acid molecules are oligonucleotides.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to identify a cell that expresses the polypeptide.

### VI. POLYPEPTIDE EXPRESSION

In some aspects, there are nucleic acid molecule encoding polypeptides, antibodies, or antigen binding fragments of the disclosure. The nucleic acid molecules may be used to express large quantities of polypeptides. If the nucleic acid molecules are derived from a non-human, non-transgenic animal, the nucleic acid molecules may be used for humanization of the antibody or TCR genes.

### A. Vectors

In some aspects, contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, or the antigen-binding portion thereof. In some aspects, expression vectors comprising nucleic acid molecules may encode fusion proteins, modified antibodies, antibody heavy and/or light chain, antibody fragments, and probes thereof. In addition to control sequences that govern transcription and translation, vectors and expression vectors may contain nucleic acid sequences that serve other functions as well.

To express the polypeptides or peptides of the disclosure, DNAs encoding the polypeptides or peptides are inserted into expression vectors such that the gene area is operatively linked to transcriptional and translational control sequences. In some aspects, a vector that encodes a functionally complete human CH or CL immunoglobulin sequence with appropriate restriction sites engineered so that any VH or VL sequence can be easily inserted and expressed. In some aspects, a vector that encodes a functionally complete human TCR alpha or TCR beta sequence with appropriate restriction sites engineered so that any variable sequence or CDR1, CDR2, and/or CDR3 can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

### B. Expression Systems

Numerous expression systems exist that comprise at least a part or all of the expression vectors discussed above. Prokaryote- and/or eukaryote-based systems can be employed for use with an embodiment to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

### C. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. No. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

### VIII. CANCER THERAPY

In some aspects, the disclosed methods comprise administering a cancer therapy to a subject or patient. The cancer therapy may be chosen based on an expression level measurements, alone or in combination with the clinical risk score calculated for the subject. The cancer therapy may be chosen based on a genotype of a subject. The cancer therapy may be chosen based on the presence or absence of one or more polymorphisms in a subject. In some aspects, the cancer therapy comprises a local cancer therapy. In some aspects, the cancer therapy excludes a systemic cancer therapy. In some aspects, the cancer therapy excludes a local therapy. In some aspects, the cancer therapy comprises a local cancer therapy without the administration of a system cancer therapy. In some aspects, the cancer therapy comprises an immunotherapy, which may be a checkpoint inhibitor therapy. Any of these cancer therapies may also be excluded. Combinations of these therapies may also be administered.

The term “cancer,” as used herein, may be used to describe a solid tumor, metastatic cancer, or non-metastatic cancer. In certain aspects, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus. In some aspects, the cancer is a Stage I cancer. In some aspects, the cancer is a Stage II cancer. In some aspects, the cancer is a Stage III cancer. In some aspects, the cancer is a Stage IV cancer.

The methods of the disclosure describe treating or preventing cancer in a subject comprising administering to the subject an antibody, antigen binding fragment, polypeptide, CAR, composition, or host cell of the disclosure. The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some aspects, the cancer is aggressive cancer. In some aspects, the cancer is Stage I cancer. In some aspects, the cancer is Stage II cancer (e.g., IIA, IIB, IIC). In some aspects, the cancer is Stage III cancer (e.g., IIIA, IIIB, IIIC). In some aspects, the cancer is Stage IV cancer (e.g., IVA, IVB).

In some aspects, disclosed are methods for treating cancer originating from the prostate. In some aspects, the cancer is prostate cancer. In some aspects, the cancer is a recurrent cancer. In some aspects, the cancer is an immunotherapy-resistant cancer.

In some aspects, disclosed are methods for treating cancer originating from the breast. In some aspects, the cancer is triple negative breast cancer.

In some aspects, disclosed are methods for treating blood cancer. In some aspects, the cancer is leukemia.

Methods may involve the determination, administration, or selection of an appropriate cancer “management regimen” and predicting the outcome of the same. As used herein the phrase “management regimen” refers to a management plan that specifies the type of examination, screening, diagnosis, surveillance, care, and treatment (such as dosage, schedule and/or duration of a treatment) provided to a subject in need thereof (e.g., a subject diagnosed with cancer).

### A. Borealin-Targeting Polypeptides

Aspects of the disclosure comprise administering one or more borealin-targeting polypeptides of the present disclosure to a subject. A subject may be administered 1, 2, 3, 4, 5, 6, 7, 8, 9, 10, or more borealin-targeting polypeptides of the disclosure. A borealin-targeting polypeptide of the disclosure may be administered alone or in combination with any other cancer therapy known in the art and/or described herein. A borealin-targeting polypeptide of the disclosure may be administered to a subject in various forms, including as a recombinant polypeptide, as a nucleic acid encoding the polypeptide, and as a cell (e.g., immune cell such as T cell or NK cell) expressing or capable of expressing the polypeptide.

### B. Radiotherapy

In some aspects, a radiotherapy, such as ionizing radiation, is administered to a subject. As used herein, “ionizing radiation” means radiation comprising particles or photons that have sufficient energy or can produce sufficient energy via nuclear interactions to produce ionization (gain or loss of electrons). A non-limiting example of ionizing radiation is x-radiation. Means for delivering x-radiation to a target tissue or cell are well known in the art.

In some aspects, the radiotherapy can comprise external radiotherapy, internal radiotherapy, radioimmunotherapy, or intraoperative radiation therapy (IORT). In some aspects, the external radiotherapy comprises three-dimensional conformal radiation therapy (3D-CRT), intensity modulated radiation therapy (IMRT), proton beam therapy, image-guided radiation therapy (IGRT), or stereotactic radiation therapy. In some aspects, the internal radiotherapy comprises interstitial brachytherapy, intracavitary brachytherapy, or intraluminal radiation therapy. In some aspects, the radiotherapy is administered to a primary tumor.

In some aspects, the amount of ionizing radiation is greater than 20 Gy and is administered in one dose. In some aspects, the amount of ionizing radiation is 18 Gy and is administered in three doses. In some aspects, the amount of ionizing radiation is at least, at most, or exactly 0.5, 1, 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 Gy (or any derivable range therein). In some aspects, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some aspects, the amount of radiotherapy administered to a subject may be presented as a total dose of radiotherapy, which is then administered in fractionated doses. For example, in some aspects, the total dose is 50 Gy administered in 10 fractionated doses of 5 Gy each. In some aspects, the total dose is 50-90 Gy, administered in 20-60 fractionated doses of 2-3 Gy each. In some aspects, the total dose of radiation is at least, at most, or about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 Gy (or any derivable range therein). In some aspects, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein). In some aspects, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some aspects, at least, at most, or exactly 1, 2, 3, 4, 5, 6, 7, 8, 9, 10, 11, or 12 (or any derivable range therein) fractionated doses are administered per day. In some aspects, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### C. Cancer Immunotherapy

In some aspects, the methods comprise administration of a cancer immunotherapy. Cancer immunotherapy (sometimes called immuno-oncology, abbreviated IO) is the use of the immune system to treat cancer. Immunotherapies can, in some cases, be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Various immunotherapies are known in the art, and certain examples are described below.

### 1. Checkpoint Inhibitors and Combination Treatment

Aspects of the disclosure may include administration of immune checkpoint inhibitors, examples of which are further described below. As disclosed herein, “checkpoint inhibitor therapy” (also “immune checkpoint blockade therapy”, “immune checkpoint therapy”, “ICT,” “checkpoint blockade immunotherapy,” or “CBI”), refers to cancer therapy comprising providing one or more immune checkpoint inhibitors to a subject suffering from or suspected of having cancer.

a. PD-1, PDL1, and PDL2 inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some aspects, PD-1, PDL1, and PDL2 are human PD-1, PDL1 and PDL2.

In some aspects, the PD-1 inhibitor is a molecule that inhibits the binding of PD-1 to its ligand binding partners. In a specific aspect, the PD-1 ligand binding partners are PDL1 and/or PDL2. In another aspect, a PDL1 inhibitor is a molecule that inhibits the binding of PDL1 to its binding partners. In a specific aspect, PDL1 binding partners are PD-1 and/or B7-1. In another aspect, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some aspects, the PD-1 inhibitor is an anti-PD-1 antibody (e.g., a human antibody, a humanized antibody, or a chimeric antibody). In some aspects, the anti-PD-1 antibody is selected from the group consisting of nivolumab, pembrolizumab, and pidilizumab. In some aspects, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some aspects, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some aspects, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some aspects, the inhibitor comprises the heavy and light chain CDRs or VRs of nivolumab, pembrolizumab, or pidilizumab. Accordingly, in one aspect, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another aspect, the antibody competes for binding with and/or binds to the same epitope on PD-1, PDL1, or PDL2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some aspects, the inhibitor blocks the CTLA-4 and B7-1 interaction. In some aspects, the inhibitor blocks the CTLA-4 and B7-2 interaction.

In some aspects, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO 01/14424).

In some aspects, the inhibitor comprises the heavy and light chain CDRs or VRs of tremelimumab or ipilimumab. Accordingly, in one aspect, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another aspect, the antibody competes for binding with and/or binds to the same epitope on PD-1, B7-1, or B7-2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

c. LAG3

Another immune checkpoint that can be targeted in the methods provided herein is the lymphocyte-activation gene 3 (LAG3), also known as CD223 and lymphocyte activating 3. The complete mRNA sequence of human LAG3 has the Genbank accession number NM_002286. LAG3 is a member of the immunoglobulin superfamily that is found on the surface of activated T cells, natural killer cells, B cells, and plasmacytoid dendritic cells. LAG3's main ligand is MHC class II, and it negatively regulates cellular proliferation, activation, and homeostasis of T cells, in a similar fashion to CTLA-4 and PD-1, and has been reported to play a role in Treg suppressive function. LAG3 also helps maintain CD8+ T cells in a tolerogenic state and, working with PD-1, helps maintain CD8 exhaustion during chronic viral infection. LAG3 is also known to be involved in the maturation and activation of dendritic cells. Inhibitors of the disclosure may block one or more functions of LAG3 activity.

In some aspects, the immune checkpoint inhibitor is an anti-LAG3 antibody (e.g., a human antibody, a humanized antibody, or a chimeric antibody), an antigen binding fragment thereof, an immunoadhesin, a fusion protein, or oligopeptide.

Anti-human-LAG3 antibodies (or VH and/or VL domains derived therefrom) suitable for use in the present methods can be generated using methods well known in the art. Alternatively, art recognized anti-LAG3 antibodies can be used. For example, the anti-LAG3 antibodies can include: GSK2837781, IMP321, FS-118, Sym022, TSR-033, MGD013, BI754111, AVA-017, or GSK2831781. The anti-LAG3 antibodies disclosed in: U.S. Pat. No. 9,505,839 (BMS-986016, also known as relatlimab); U.S. Pat. No. 10,711,060 (IMP-701, also known as LAG525); U.S. Pat. No. 9,244,059 (IMP731, also known as H5L7BW); U.S. Pat. No. 10,344,089 (25F7, also known as LAG3.1); WO 2016/028672 (MK-4280, also known as 28G-10); WO 2017/019894 (BAP050); Burova E., et al., J. ImmunoTherapy Cancer, 2016; 4(Supp. 1):P195 (REGN3767); Yu, X., et al., mAbs, 2019; 11:6 (LBL-007) can be used in the methods disclosed herein. These and other anti-LAG-3 antibodies useful in the claimed invention can be found in, for example: WO 2016/028672, WO 2017/106129, WO 2017062888, WO 2009/044273, WO 2018/069500, WO 2016/126858, WO 2014/179664, WO 2016/200782, WO 2015/200119, WO 2017/019846, WO 2017/198741, WO 2017/220555, WO 2017/220569, WO 2018/071500, WO 2017/015560; WO 2017/025498, WO 2017/087589, WO 2017/087901, WO 2018/083087, WO 2017/149143, WO 2017/219995, US 2017/0260271, WO 2017/086367, WO 2017/086419, WO 2018/034227, and WO 2014/140180. The teachings of each of the aforementioned publications are hereby incorporated by reference. Antibodies that compete with any of these art-recognized antibodies for binding to LAG3 also can be used.

In some aspects, the inhibitor comprises the heavy and light chain CDRs or VRs of an anti-LAG3 antibody. Accordingly, in one aspect, the inhibitor comprises the CDR1, CDR2, and CDR3 domains of the VH region of an anti-LAG3 antibody, and the CDR1, CDR2 and CDR3 domains of the VL region of an anti-LAG3 antibody. In another aspect, the antibody has at least about 70, 75, 80, 85, 90, 95, 97, or 99% (or any derivable range therein) variable region amino acid sequence identity with the above-mentioned antibodies.

d. TIM-3

Another immune checkpoint that can be targeted in the methods provided herein is the T-cell immunoglobulin and mucin-domain containing-3 (TIM-3), also known as hepatitis A virus cellular receptor 2 (HAVCR2) and CD366. The complete mRNA sequence of human TIM-3 has the Genbank accession number NM_032782. TIM-3 is found on the surface IFNγ-producing CD4+ Th1 and CD8+ Tc1 cells. The extracellular region of TIM-3 consists of a membrane distal single variable immunoglobulin domain (IgV) and a glycosylated mucin domain of variable length located closer to the membrane. TIM-3 is an immune checkpoint and, together with other inhibitory receptors including PD-1 and LAG3, it mediates the T-cell exhaustion. TIM-3 has also been shown as a CD4+ Th1-specific cell surface protein that regulates macrophage activation. Inhibitors of the disclosure may block one or more functions of TIM-3 activity.

In some aspects, the immune checkpoint inhibitor is an anti-TIM-3 antibody (e.g., a human antibody, a humanized antibody, or a chimeric antibody), an antigen binding fragment thereof, an immunoadhesin, a fusion protein, or oligopeptide.

Anti-human-TIM-3 antibodies (or V_{H} and/or V_{L} domains derived therefrom) suitable for use in the present methods can be generated using methods well known in the art. Alternatively, art recognized anti-TIM-3 antibodies can be used. For example, anti-TIM-3 antibodies including: MBG453, TSR-022 (also known as Cobolimab), and LY3321367 can be used in the methods disclosed herein. These and other anti-TIM-3 antibodies useful in the claimed invention can be found in, for example: U.S. Pat. Nos. 9,605,070, 8,841,418, US2015/0218274, and US 2016/0200815. The teachings of each of the aforementioned publications are hereby incorporated by reference. Antibodies that compete with any of these art-recognized antibodies for binding to TIM-3 also can be used.

In some aspects, the inhibitor comprises the heavy and light chain CDRs or VRs of an anti-TIM-3 antibody. Accordingly, in one aspect, the inhibitor comprises the CDR1, CDR2, and CDR3 domains of the V_{H} region of an anti-TIM-3 antibody, and the CDR1, CDR2 and CDR3 domains of the V_{L} region of an anti-TIM-3 antibody. In another aspect, the antibody has at least about 70, 75, 80, 85, 90, 95, 97, or 99% (or any derivable range or value therein) variable region amino acid sequence identity with the above-mentioned antibodies.

### 2. Activator of Co-Stimulatory Molecules

In some aspects, the immunotherapy comprises an activator (also “agonist”) of a co-stimulatory molecule. In some aspects, the agonist comprises an agonist of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Agonists include activating antibodies, polypeptides, compounds, and nucleic acids.

### 3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

### 4. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell, natural killer (NK) cell, or other immune cell. The receptors are called chimeric because they are fused of parts from different sources. CAR-T cell therapy refers to a treatment that uses such transformed cells for cancer therapy, where the transformed cells are T cells. Similar therapies include, for example, CAR-NK cell therapy, which uses transformed NK cells.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Example CAR-T therapies include Tisagenlecleucel (Kymriah) and Axicabtagene ciloleucel (Yescarta).

### 5. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an example interleukin cytokine therapy.

### 6. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

### D. Oncolytic Virus

In some aspects, the cancer therapy comprises an oncolytic virus. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses for long-term immunotherapy

### E. Chemotherapies

In some aspects, a therapy of the present disclosure comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some aspects, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection.

Other suitable chemotherapeutic agents include antimicrotubule agents, e.g., Paclitaxel (“Taxol”) and doxorubicin hydrochloride (“doxorubicin”). Doxorubicin is absorbed poorly and is preferably administered intravenously. In certain aspects, appropriate intravenous doses for an adult include about 60 mg/m² to about 75 mg/m² at about 21-day intervals or about 25 mg/m² to about 30 mg/m² on each of 2 or 3 successive days repeated at about 3 week to about 4 week intervals or about 20 mg/m² once a week.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred in certain cases. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

The amount of the chemotherapeutic agent delivered to a patient may be variable. In one suitable aspect, the chemotherapeutic agent may be administered in an amount effective to cause arrest or regression of the cancer in a host, when the chemotherapy is administered with the construct. In other aspects, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### F. Hormone Therapy

In some aspects, a cancer therapy of the present disclosure is a hormone therapy. In particular aspects, a prostate cancer therapy comprises hormone therapy. Various hormone therapies are known in the art and contemplated herein. Examples of hormone therapies include, but are not limited to, luteinizing hormone-releasing hormone (LHRH) analogs, LHRH antagonists, androgen receptor antagonists, and androgen synthesis inhibitors.

### G. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present aspects, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### H. Radiotherapy

In some aspects, a cancer therapy (e.g., prostate cancer therapy) comprises radiation, such as ionizing radiation (IR). As used herein, “ionizing radiation” means radiation comprising particles or photons that have sufficient energy or can produce sufficient energy via nuclear interactions to produce ionization (gain or loss of electrons). An example ionizing radiation is an x-radiation. Various means for delivering x-radiation to a target tissue or cell are well known in the art and.

In some aspects, the amount of ionizing radiation is greater than 20 Gy and is administered in one dose. In some aspects, the amount of ionizing radiation is 18 Gy and is administered in three doses. In some aspects, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some aspects, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some aspects, the amount of IR may be presented as a total dose of IR, which is then administered in fractionated doses. For example, in some aspects, the total dose is 50 Gy administered in 10 fractionated doses of 5 Gy each. In some aspects, the total dose is 50-90 Gy, administered in 20-60 fractionated doses of 2-3 Gy each. In some aspects, the total dose of IR is at least, at most, or about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some aspects, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some aspects, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some aspects, at least, at most, or exactly 1, 2, 3, 4, 5, 6, 7, 8, 9, 10, 11, or 12 (or any derivable range therein) fractionated doses are administered per day. In some aspects, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### I. Additional Cancer Therapies

Therapeutic methods disclosed herein may comprise one or more additional cancer therapies. A cancer therapy of the disclosure may comprise, for example, cryoablative therapy, high-intensity ultrasound (also “high-intensity focused ultrasound”), photodynamic therapy, laser ablation, and/or irreversible electroporation. A cancer therapy of the disclosure may comprise 1, 2, 3, 4, 5, or more distinct therapeutic methods.

It is contemplated that a cancer treatment may exclude any of the cancer treatments described herein. Furthermore, aspects of the disclosure include patients that have been previously treated for a therapy described herein, are currently being treated for a therapy described herein, or have not been treated for a therapy described herein. In some aspects, the patient is one that has been determined to be resistant to a therapy described herein. In some aspects, the patient is one that has been determined to be sensitive to a therapy described herein.

### IX. PHARMACEUTICAL COMPOSITIONS

The present disclosure includes methods for treating disease and modulating immune responses in a subject in need thereof. The disclosure includes cells that may be in the form of a pharmaceutical composition that can be used to induce or modify an immune response.

Administration of the compositions according to the current disclosure will typically be via any common route. This includes, but is not limited to parenteral, orthotopic, intradermal, subcutaneous, orally, transdermally, intramuscular, intraperitoneal, intraperitoneally, intraorbitally, by implantation, by inhalation, intraventricularly, intranasally or intravenous injection. In some embodiments, compositions of the present disclosure (e.g., compositions comprising borealin-binding polypeptides) are administered to a subject intravenously.

Typically, compositions and therapies of the disclosure are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner.

The manner of application may be varied widely. Any of the conventional methods for administration of pharmaceutical compositions comprising cellular components are applicable. The dosage of the pharmaceutical composition will depend on the route of administration and will vary according to the size and health of the subject.

In many instances, it will be desirable to have multiple administrations of at most or at least 3, 4, 5, 6, 7, 8, 9, 10 or more. The administrations may range from 2-day to 12-week intervals, more usually from one to two week intervals.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. The pharmaceutical compositions of the current disclosure are pharmaceutically acceptable compositions.

The compositions of the disclosure can be formulated for parenteral administration, e.g., formulated for injection via the intravenous, intramuscular, sub-cutaneous, or even intraperitoneal routes. Typically, such compositions can be prepared as injectables, either as liquid solutions or suspensions and the preparations can also be emulsified.

Pharmaceutical forms suitable for injectable use include sterile aqueous solutions or dispersions; formulations including sesame oil, peanut oil, or aqueous propylene glycol. It also should be stable under the conditions of manufacture and storage and must be preserved against the contaminating action of microorganisms, such as bacteria and fungi.

Sterile injectable solutions are prepared by incorporating the active ingredients (e.g., polypeptides of the disclosure) in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

An effective amount of a composition is determined based on the intended goal. The term “unit dose” or “dosage” refers to physically discrete units suitable for use in a subject, each unit containing a predetermined quantity of the composition calculated to produce the desired responses discussed herein in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

The compositions and related methods of the present disclosure, particularly administration of a composition of the disclosure may also be used in combination with the administration of additional therapies such as the additional therapeutics described herein or in combination with other traditional therapeutics known in the art.

The therapeutic compositions and treatments disclosed herein may precede, be co-current with and/or follow another treatment or agent by intervals ranging from minutes to weeks. In embodiments where agents are applied separately to a cell, tissue or organism, one would generally ensure that a significant period of time did not expire between the time of each delivery, such that the therapeutic agents would still be able to exert an advantageously combined effect on the cell, tissue or organism. For example, in such instances, it is contemplated that one may contact the cell, tissue or organism with two, three, four or more agents or treatments substantially simultaneously (i.e., within less than about a minute). In other aspects, one or more therapeutic agents or treatments may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering another therapeutic agent or treatment.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In some embodiments, the therapeutically effective or sufficient amount of the immune checkpoint inhibitor, such as an antibody and/or microbial modulator, that is administered to a human will be in the range of about 0.01 to about 50 mg/kg of patient body weight whether by one or more administrations. In some embodiments, the therapy used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In one embodiment, a therapy described herein is administered to a subject at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 M or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### X. DETECTABLE LABELS

In some aspects of this disclosure, it will be useful to detectably or therapeutically label a Fab polypeptide or protein G Fab-binding domain. Methods for conjugating polypeptides to these agents are known in the art. For the purpose of illustration only, polypeptides can be labeled with a detectable moiety such as a radioactive atom, a chromophore, a fluorophore, or the like. Such labeled polypeptides can be used for diagnostic techniques, either in vivo, or in an isolated test sample or in methods described herein.

As used herein, the term “label” intends a directly or indirectly detectable compound or composition that is conjugated directly or indirectly to the composition to be detected, e.g., polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition that is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6.sup.th ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6.sup.th ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

Attachment of the fluorescent label may be either directly to the cellular component or compound or alternatively, can by via a linker. Suitable binding pairs for use in indirectly linking the fluorescent label to the intermediate include, but are not limited to, antigens/polypeptides, e.g., rhodamine/anti-rhodamine, biotin/avidin and biotin/strepavidin.

The coupling of polypeptides to low molecular weight haptens can increase the sensitivity of the antibody in an assay. The haptens can then be specifically detected by means of a second reaction. For example, it is common to use haptens such as biotin, which reacts avidin, or dinitrophenol, pyridoxal, and fluorescein, which can react with specific anti-hapten polypeptides. See, Harlow and Lane (1988) supra.

### XI. SAMPLE PREPARATION

In certain aspects, methods involve obtaining or evaluating a sample from a subject. The sample may include a sample obtained from any source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain embodiments the samples are obtained by biopsy. In other embodiments the sample is obtained by swabbing, endoscopy, scraping, phlebotomy, or any other methods known in the art. In some cases, the sample may be obtained, stored, or transported using components of a kit of the present methods. In some cases, multiple samples, such as multiple esophageal samples may be obtained for diagnosis by the methods described herein. In other cases, multiple samples, such as one or more samples from one tissue type (for example esophagus) and one or more samples from another specimen (for example serum) may be obtained for diagnosis by the methods. In some cases, multiple samples such as one or more samples from one tissue type (e.g. esophagus) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one embodiment, the sample is a fine needle aspirate of a esophageal or a suspected esophageal tumor or neoplasm. In some cases, the fine needle aspirate sampling procedure may be guided by the use of an ultrasound, X-ray, or other imaging device.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some embodiments, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### XII. HOST CELLS

As used herein, the terms “cell,” “cell line,” and “cell culture” may be used interchangeably. All of these terms also include both freshly isolated cells and ex vivo cultured, activated or expanded cells. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

In certain embodiments transfection can be carried out on any prokaryotic or eukaryotic cell. In some aspects electroporation involves transfection of a human cell. In other aspects electroporation involves transfection of an animal cell. In certain aspects transfection involves transfection of a cell line or a hybrid cell type. In some aspects the cell or cells being transfected are cancer cells, tumor cells or immortalized cells. In some instances tumor, cancer, immortalized cells or cell lines are induced and in other instances tumor, cancer, immortalized cells or cell lines enter their respective state or condition naturally. In certain aspects the cells or cell lines can be A549, B-cells, B16, BHK-21, C2C12, C6, CaCo-2, CAP/, CAP-T, CHO, CHO2, CHO-DG44, CHO-K1, COS-1, Cos-7, CV-1, Dendritic cells, DLD-1, Embryonic Stem (ES) Cell or derivative, H1299, HEK, 293, 293T, 293FT, Hep G2, Hematopoietic Stem Cells, HOS, Huh-7, Induced Pluripotent Stem (iPS) Cell or derivative, Jurkat, K562, L5278Y, LNCaP, MCF7, MDA-MB-231, MDCK, Mesenchymal Cells, Min-6, Monocytic cell, Neuro2a, NIH 3T3, NIH3T3L1, K562, NK-cells, NS0, Panc-1, PC12, PC-3, Peripheral blood cells, Plasma cells, Primary Fibroblasts, RBL, Renca, RLE, SF21, SF9, SH-SY5Y, SK-MES-1, SK-N-SH, SL3, SW403, Stimulus-triggered Acquisition of Pluripotency (STAP) cell or derivate SW403, T-cells, THP-1, Tumor cells, U2OS, U937, peripheral blood lymphocytes, expanded T cells, hematopoietic stem cells, or Vero cells.

### XIII. KITS

Certain aspects of the present invention also concern kits containing compositions of the disclosure or compositions to implement methods of the disclosure. In some embodiments, kits can be used to detect the presence of a cancer cell in a sample. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, a kit contains one or more polypeptides capable of binding to borealin, including polypeptides disclosed herein. For example, a kit may comprise at least 1, 2, 3, 4, 5, 6, 7, 8, 9, 10 or more antibodies or antibody fragments (e.g., Fabs) disclosed herein for detecting borealin, in some cases for detecting borealin on a surface of a cancer cell. In some embodiments, a kit comprises a detection pair. In some embodiments, a kit comprises an enzyme. In some embodiments, a kit comprises a substrate for an enzyme.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. In certain aspects, negative and/or positive control nucleic acids, probes, and inhibitors are included in some kit embodiments.

Kits may further comprise instructions for use. For example, in some embodiments, a kit comprises instructions for detecting borealin, such as borealin on a surface of a cancer cell, in a sample.

It is contemplated that any method or composition described herein can be implemented with respect to any other method or composition described herein and that different embodiments may be combined. The claims originally filed are contemplated to cover claims that are multiply dependent on any filed claim or combination of filed claims.

### XIV. EXAMPLES

The following examples are included to demonstrate certain embodiments of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### Example Overview

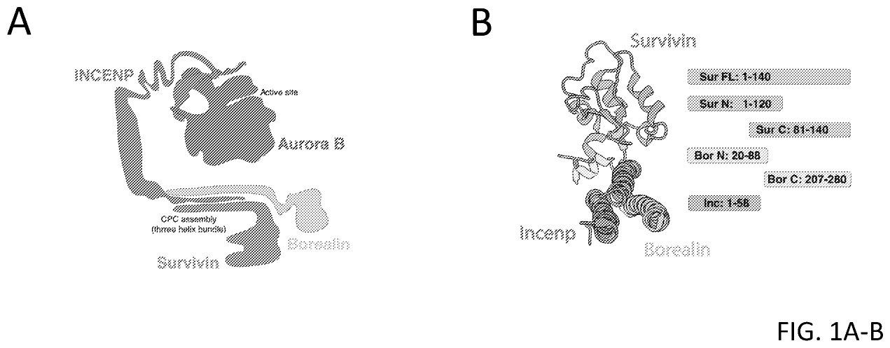

A large panel of fragments of antibodies (Fabs) to borealin was generated using phage display mutagenesis. The borealin antigens used in the selections included: the N-terminal domain and C-terminal domains (FIG. 1). The affinities of these Fabs ranged from K_{D}˜1 nM-30 nM. These Fabs were used as imaging agents to detect borealin. Surprisingly, it was also found several of these Fabs could bind to the cell surface of various cancer cell lines. Importantly, they did not bind to normal cells (PBMCs). Notably, the phenomenon of borealin Fabs binding to the cell surface did not correlate to affinity alone, there appeared to a spatial component that is both epitope and orientation sensitive.

It was found that by inducing cellular stress by: i) protein over-expression, ii) anticancer drug treatment and iii) radiation exposure, the levels of cell surface borealin were significantly increased. To evaluate whether the level of borealin surface expression was robust and stable enough to be exploited in immunotherapeutic applications, a borealin Fab BiTE (bi-specific T-Cell engager) was engineered, where one arm was a borealin Fab and the other arm was the single chain version of the CD3 antibody, OKT3. Several different BiTE formats were generated. The idea was that the CD3 arm would engage and activate T-cells in the PBMCs and the borealin Fab would bind to cell surface borealin that was expressed on the antigen presenting cell (APC). For these experiments, the effects on several cancer cell lines were tested. It was shown that cell killing (LDH release assay) and cytokine release (IL2, INF-G) could be achieved in a dose dependent fashion. Antibody-drug conjugates (ADCs) were generating from certain borealin Fabs and tested against SKBR3 cancer cells. It was shown that cell killing (LDH release assay) could be achieved. Robust cell killing was also demonstrated using CAR-T cell format. Examples of the methods to obtain these results are provided below.

### Example 1: Expression and Purification of the Targets

The Borealin antigen included the N-terminal domain (20-88) (Bor-N) and the C-terminal domain (207-280) (Bor-C) (1). These were based on previous structure and biochemical analysis to ensure that the constructs would be folded and stable as an individual domain (FIG. 1). These antigens of interest harboring His- and the Avi-tags were overexpressed in E. coli BL21(DE3) cells co-transformed with the E. coli biotin ligase BirA to obtain biotinylated protein. The overexpressed proteins were purified by Ni-NTA chromatography and the extent of biotinylation were determined by Streptavidin (SA) pull-down assay. This assay was performed using SA magnetic beads (#Z5482, Promega) at RT and all incubation steps were for 15 min. 3 g of target was incubated with 100 μL of SA magnetic beads with gentle mixing. Unbound protein fraction (flow-through) was collected, and beads washed three times. The extent of biotinylation was analyzed by comparing the amount of protein in the different fractions by SDS-PAGE.

### Example 2: Phage Display Selections for Generation of Target Specific Fabs

To obtain Fabs, a selection campaign was performed on biotinylated borealin targets using library E (1). The panning was done at room temperature following published procedures (2). In the first round, 1 ml of phage library E containing 1012 virions was added to SA magnetic beads (Promega) pre-coated with 200 nM of biotinylated target. The phage pool was incubated with the beads for 30 mins. Beads containing bound virions after extensive washing were used to infect freshly grown log-phase E. coli XL1-Blue cells. Phages were amplified overnight in 2YT media with 50 μg/ml Amp and 109 plaque-forming units/ml M13-K07 helper phage. To increase the stringency of selection, three additional rounds of sorting were performed by decreasing the target concentration in each round (second round—100 nM; third round—50 nM; fourth round—10 nM), using the amplified pool of virions of the preceding round as the input. Sorting from the second to fourth rounds was done on a Kingfisher instrument. From the second round onward, 0.1 M glycine; pH 2.7 was used to elute the bound phage from the immobilized target. To eliminate the potential SA binders, the amplified phage pool after each round was negatively selected against SA beads before using as input for the next round.

### Example 3: Phage ELISA

A single-point competitive phage ELISA was used to rapidly estimate the affinities of the obtained Fabs in phage format (3). Colonies of E. coli XL1-Blue harboring phagemids were inoculated directly into 500 μl of 2YT broth supplemented with 100 μg/ml Ampicillin and M13-KO7 helper phage. The cultures were grown at 37° C. for 16-20 hours at 280 rpm in a 96 deep-well block plate. 25 nM biotinylated target was immobilized on ELISA plates coated with neutravidin and blocked with 0.5% BSA in TBS. Culture supernatants containing Fab-phage were diluted ten-fold in TBST buffer (TBS buffer containing 0.05% (v/v) Tween-20) with or without 50 nM target proteins as soluble competitor in a 96 well plate. After 15 minutes incubation at room temperature, the mixtures were transferred to ELISA plates containing the immobilized target. The ELISA plates were incubated with the phage-competitor mixture for another 15 minutes and then washed with TBST. The washed ELISA plates were incubated with HRP conjugated anti-M13 mouse monoclonal antibody (1:5000 dilution in TBST buffer) for 30 minutes. The plates were again washed with TBST, developed with TMB substrate, quenched with 1.0 M HCl, and absorbance (A450) determined spectrophotometrically at 450 nm. For each clone, the ratio of A450 in the presence of 50 nM competitor to that in absence of competitor (competition ratio) gives the fraction of Fab-phage uncomplexed with soluble competitor. DNA of the clones with high ELISA signal and low competition ratio were sequenced to identify the number of unique binders.

### Example 4: Cloning, Expression and Purification of Fabs

Positive clones selected based on a phage ELISA were sequenced at DNA Sequencing Facility at The University of Chicago. Unique clones were reformatted in pRH2.2, an IPTG inducible vector for bacterial expression of Fabs. E. coli BL21 (Gold) cells were transformed with sequence-verified clones of Fabs in pRH2.2. Fabs were grown in 2YT media with 100 g/mL ampicillin at 37° C. for 2-2.5 h during which OD600 reached 0.6-0.8, induced with 1 mM IPTG and further grown for 4.5 h at 37° C. The Fabs were purified using affinity (protein A/protein L) followed by ion-exchange chromatography using an automated program on AKTA explorer system (3). Purified Fabs were dialyzed overnight against 20 mM HEPES; pH 7.5, 150 mM NaCl. The quality of purified Fabs was analyzed by SDS-PAGE.

### Example 5: Protein (Fab) ELISA

### A. Single Point Protein ELISA

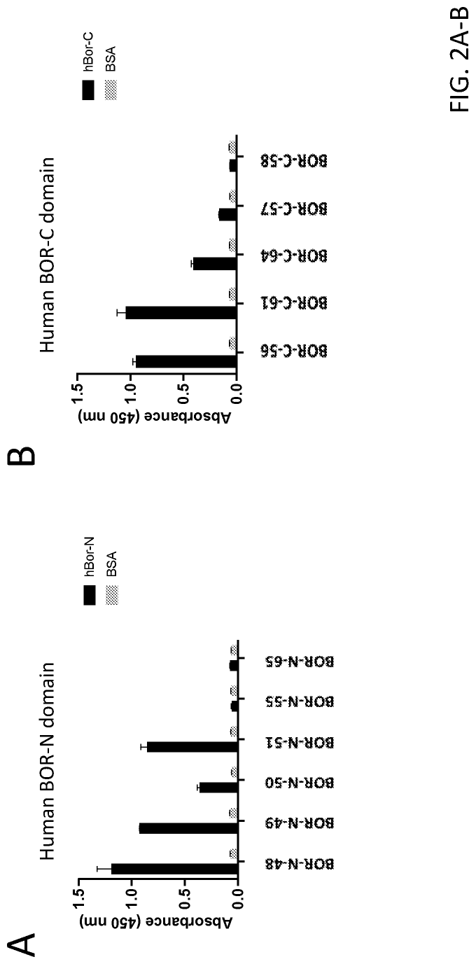

To rapidly reconfirm the binding and specificity of the obtained Fabs after reformatting into the expression vectors single-point Fab ELISA was performed (FIG. 2). Briefly, 50 nM Avi-tagged targets were immobilized on the prepared NAV-coated and blocked with 0.5% BSA in PBS 96-well ELISA plates. The target coated plates were washed with appropriate buffer (TBST or PBST) and were incubated with 200 nM Fab for 20 minutes and then washed. The washed plates were incubated with protein L conjugated with HRP (1:5000 diluted in TBST) for 30 minutes. Plates were washed and the reaction was developed using the TMB chromogenic substrate (Thermofisher) and subsequently quenched with 1M HCl. Absorbance was read at 450 nm on Epoch plate reader.

### B. Multipoint Protein ELISA

Protein based multipoint ELISA was performed to estimate the affinity of the generated unique Fabs to their cognate antigens. A fixed concentration of the immobilized target (50 nM) on ELISA plate was incubated with 3-fold serial dilutions of the purified Fabs starting from 1 μM for 15 min. The plates were washed and the bound antigen-Fab complexes were incubated with HRP-conjugated protein L (1:5000 dilution in PBST). As with phage ELISA, the plates were again washed, developed with TMB substrate and quenched with 1.0 M HCl, and absorbance (A450) was determined. To determine the affinity, the data were fitted in a non-linear sigmoidal function with variable slope in GraphPad PRISM and EC50 value was calculated.

### Example 6: Binding Affinity and Kinetics by Surface Plasmon Resonance (SPR)

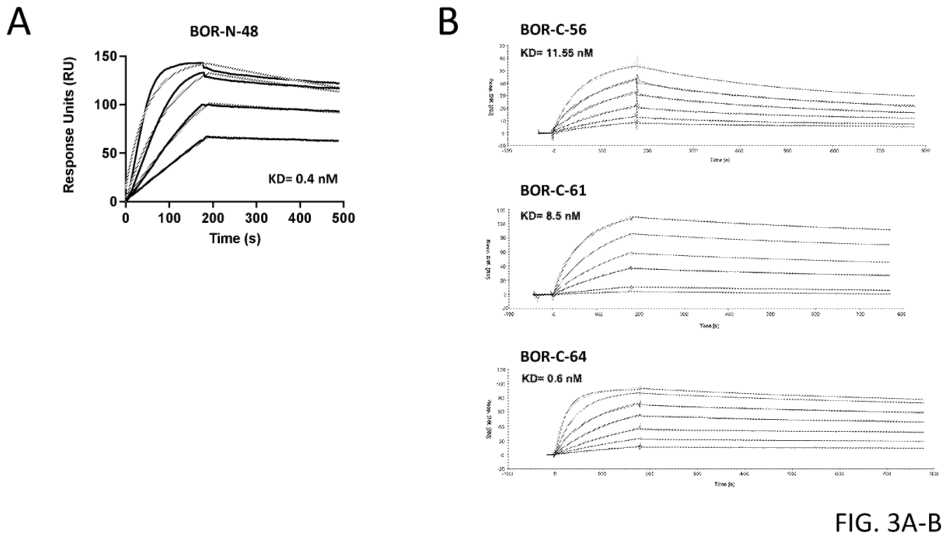

The targets were immobilized onto a nitrilotriacetic acid (NTA) sensor chip via His-tag. 2-fold serial dilutions of the Fab were injected following ligand immobilization on the sensor chip. For each kinetic assay at least five dilutions of the Fab were tested. The data were fitted using 1:1 Langmuir Model to obtain the on-rate, off-rate and the affinity (KD) of the Fab-target interaction. SPR binding curves for selected Fabs are shown in FIG. 3.

### Example 7: Flow Cytometry Staining of Tumor Cell Lines and Human Peripheral Blood Mononuclear Cells (PBMCs)

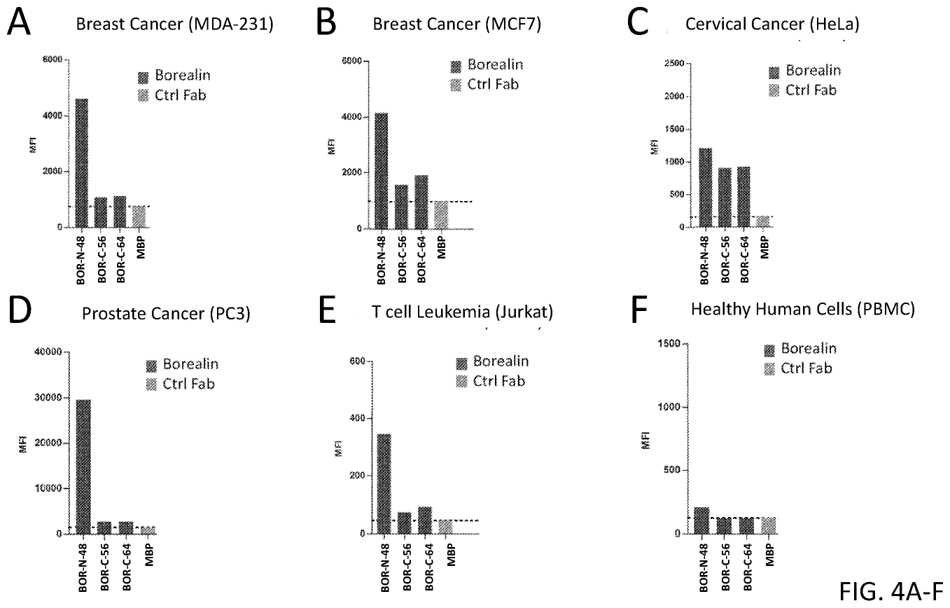

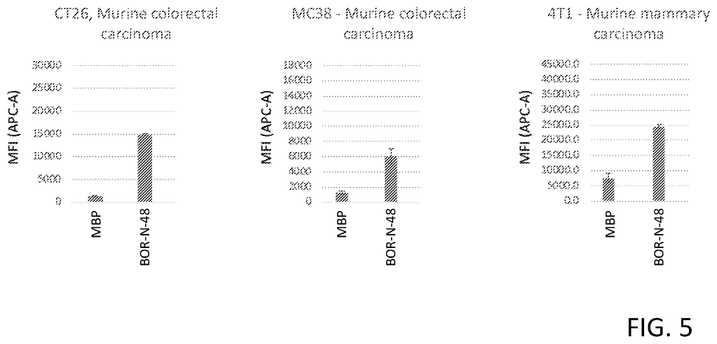

Human or murine tumor cells were trypsinized, washed and resuspended in Flow buffer (1×PBS supplemented with 2% FBS) and added to a 96-well plate (#3799, Costar). 200 nM primary CPC Fab was mixed with 0.5M cells and incubated at 37 C for 20 min. Cells were washed with Flow buffer and stained with anti-human-FAB′2-A647 secondary antibody (#109-605-006, Jackson, 1:200) for 20 min at room temperature and washed again. Samples were analyzed by flow cytometry using CytoFLEX (Beckman Coulter) and mean fluorescence intensity (MFI) data was extracted using the FlowJo software (FIG. 4A-E, FIG. 5). Human peripheral blood mononuclear cells (PBMCs) derived from a single patient were used as a source of healthy human cells. PBMCs were counted, split and spun down 400×g 5 min. Cells were resuspended in Flow buffer (1×PBS supplemented with 2% FBS) and pre-incubated with TruStain FcX (1:20, Biolegend 422302) for 10 min at 37 C, followed by 200 nM primary Fab incubation at 37 for 20 min. Cells were washed twice and stained with secondary antibody (Alexa Fluor 647 AffiniPure Goat Anti-Human IgG, F(ab′)2 fragment specific, #109-605-006, Jackson, 1:200), for 20 min at RT, washed twice and analyzed by using CytoFLEX (Beckman Coulter). Mean fluorescence intensity (MFI) data was extracted using the FlowJo software (FIG. 4F).

### Example 8: Inducing Stress Response in Cancer Cells

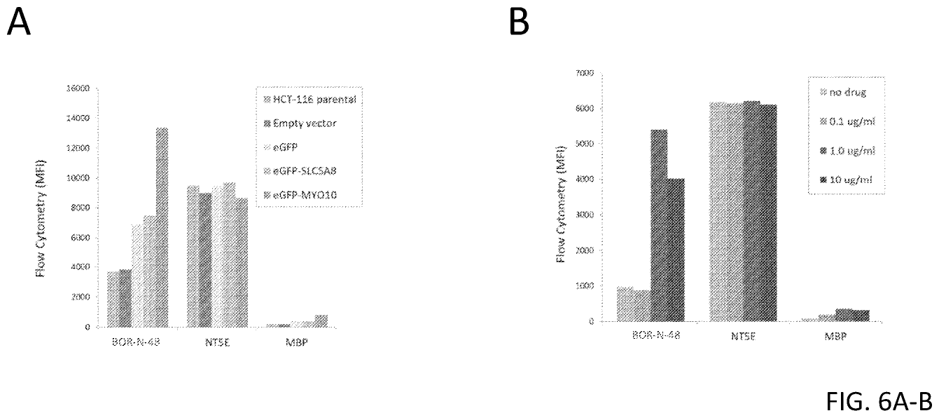

The human colon cancer cell line (HCT-116) was electroporated with SLC5A8 or MYOX fused to eGFP, which had been cloned into a mammalian expression vectors (p-EGFP-C2), using the following conditions: 1530V (voltage), 20 ms puls with and 1 pulse number. 25 ug DNA were used per 10 mm plate (FIG. 6A). Parental HCT-116 cells and protein over-expressing cells were analysed for borealin surface expression by Flow cytometr (FIG. 6A).

The human colon cancer cell line (HCT-116) was treated with 5-fluorouracil (5-FU), at the indicated concentrations, for 24 h. Cells were collected and Borealin surface expression was determined by Flow cytometry (FIG. 6B).

### Example 9: Tumor Cell Killing Measured by LDH Release and T Cell Activation

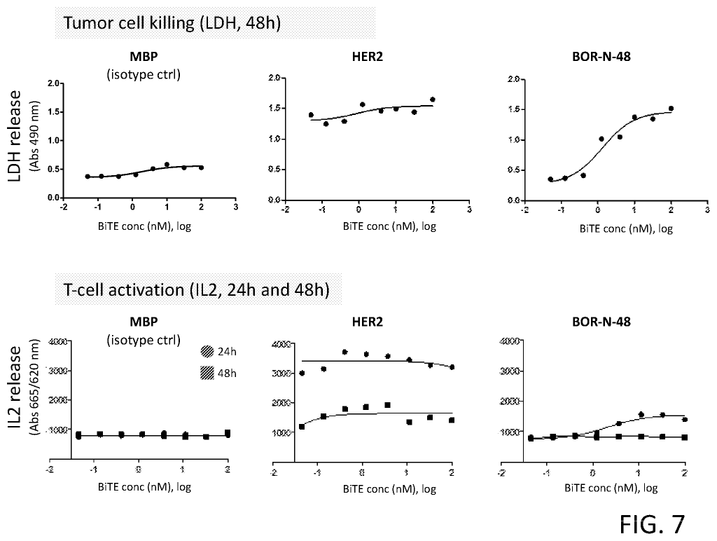

Human cancer cells (SKBR3, ATCC) were cultured according to ATCC protocols and seeded in a 96-well one day before the experiment. Human peripheral blood mononuclear cells (PBMCs) were isolated from healthy patient blood and stored frozen in liquid nitrogen. Human bi-specific T cell engagers (at indicated concentrations) were mixed with target and effector cells (1:10). After 48 h co-culturing, the medium was removed and analyzed using commercially available kits for cell lysis/LDH release (CytoTox96, Promega #G1781) and T cell activation by IL2 release (IL2Cisbio #62HIL02PEG) following the manufactures protocols (FIG. 7). The human “plug-and-play” BiTE design, in FIG. 7, has been described in Slezak et.al., (2020) Prot Sci 29(1):141-156.

### Example 10: Tumor Cell Apoptosis Measured by Caspase-3/7 Activation (IncuCyte)

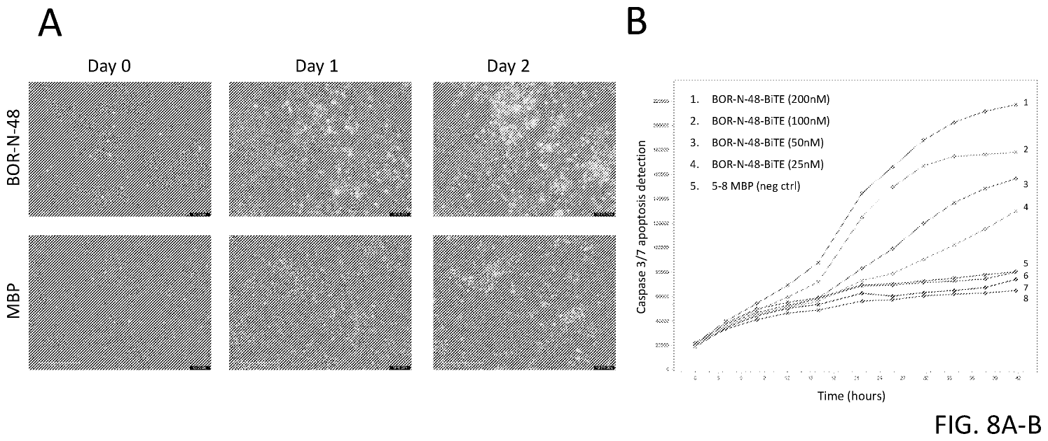

Tumor cell apoptosis were monitored over a 48-h time period by InCucyte, after treatment of murine tumor cells (4T1) with bi-specific T cell engages, in co-culture with murine T cells (FIG. 8). CellEvent™ Caspase-3/7 Green reagent (C10423, ThermoFisher) were added to the co-culture to measure apoptosis. Apoptotic cells with activated Caspase-3/7 will show bright green nuclei, while cells without activated caspase 3/7 exhibit minimal fluorescence signal. Representative images taken by the InCucyte at Day 0, 1 and 2 are shown in FIG. 8A. Graphs illustrating BiTE dose-response curves are shown in FIG. 8B. The murine 2C11-BiTE design, in FIG. 8, has been described in Stone et.al., (2012) OncoImmunology 1:6; 863-873.

### Example 11: CAR-T Tumor Cell Killing Measured by LDH Release and T Cell Activation

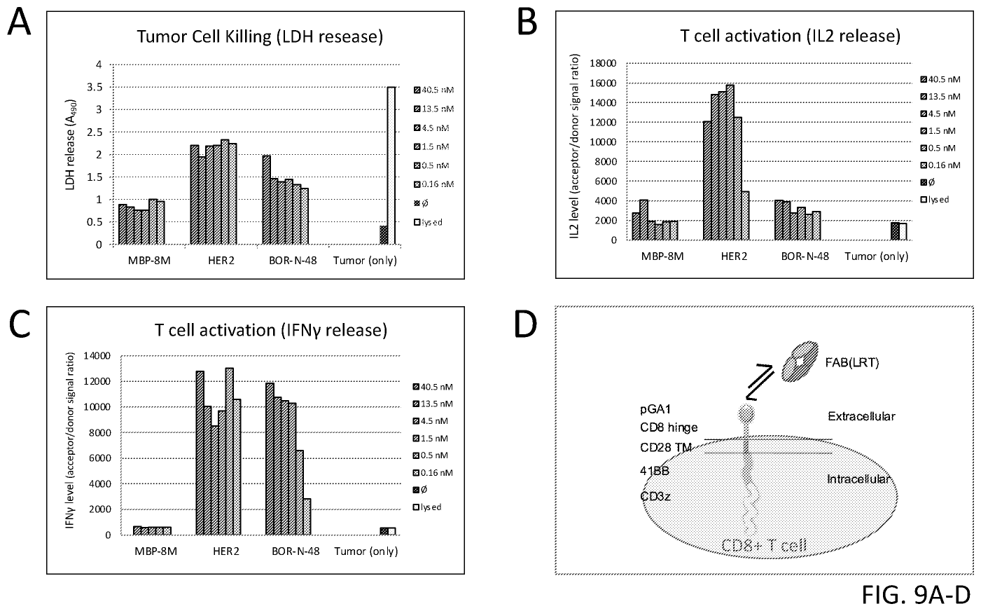

Human Chimeric Antigen Receptor T (CAR-T) cells were developed in the Kossiakoff laboratory (Arauz et.al., manuscript in preparation, FIG. 9D). This new CAR-T platform is based on an engineered protein G variant (GA1) and a novel Fab scaffold (LRT) that present exquisite specificity and selectivity on antibody capture. The CAR-T cells were characterized by Flow cytometry to ensure that GAlCAR were expressed properly on the surface of human CD8+ T cells isolated from human peripheral blood mononuclear cells. The GAlCAR cells were incubated with Borealin FAB (LRT) and human tumor cells (HCC-1954). After 24 h co-culturing, the medium was removed and analyzed using commercially available kits for cell lysis/LDH release (CytoTox96, Promega #G1781) and T cell activation by IFNg (Cisbio #62HIFNGPEG) and IL2 release (IL2Cisbio #62HIL02PEG) following the manufactures protocols (FIG. 9).

### Example 12: Fabs that can be Used in Western Blot Applications

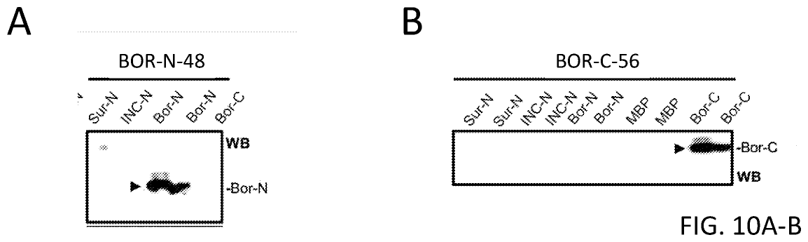

A few of the Fabs were shown to function as Western blot reagents. Purified CPC proteins (i.e. Borealin, INCENP and Survivin), collected in standard RIPA buffer, were separated on 15% gradient SDS-PAGE gels (Biorad) according to standard protocols, at 100 V. Proteins were then transferred onto methanol-activated PVDF membranes using wet transfer system (Biorad) in the transfer buffer supplemented with 20% MeOH without the addition of SDS for 1.5 hours at 100 V and 4 C. Membranes were stained with Ponceau S dye to verify correct transfer of the proteins, were briefly washed with ddH2O and blocked with 4% skimmed milk in TBS-T containing 0.05% Tween20 for 30 min. at RT on the shaker. Blocking solution was discarded and membranes were incubated with Borealin Fabs as primary detection reagents at concentration 5-10 lg/ml, in blocking buffer at 4 C O/N on the shaker. Membranes were washed 3 times with TBS-T and incubated with secondary anti-human FAB′2 specific HRP-conjugated IgG (diluted 1:10,000 in blocking buffer) for 45 minutes at RT, shaking. Membranes were washed again 3× with TBS-T, developed with ECL chemiluminescent substrate and visualized with the Biorad imaging instrument (FIG. 10).

### Example 13: Immunofluorescence (IF) Microscopy Application

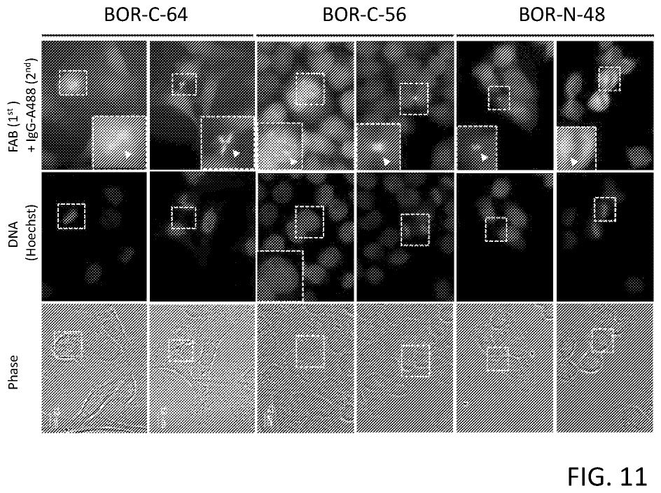

HeLa cancer cells were grown in high glucose Dublecco's modified Eagle's medium on 18 mm coverslips in 12 well plates overnight until they reached 70%-90% confluence. Cells were fixed in 4% paraformaldehyde (PFA) for 20 minutes at room temperature. Fixed cells were washed with PBS, permeabilized with 0.1% Triton X-100, washed three times with PBS and blocked in blocking buffer (PBS-T with 2% BSA) for 30 minutes. Fabs generated against the human Borealin protein were used as primary antibodies and incubated with permeabilized cells overnight at 4° C. (final concentration of 2.5-15 μg/ml in the blocking buffer). Cells were washed three times with PBS-T. Secondary AlexaFluor 488 conjugated goat anti-human IgG (final dilution 1:1000 in blocking buffer) were incubated with cells in the presence of DNA staining dye Hoechst 33342 (1:1000 dilution) for 1-2 hours at room temperature. Cells were washed three times with PBS-T, gradually dehydrated with ethanol, and the coverslips were mounted on slides in ProLong Gold antifade reagent. Images were captured using Andor DL-6584-TIL camera connected to the inverted Zeiss Axio Observer A1 epi-fluorescence microscope with X63 oil-immersion objective and the acquisition software Andor Solis. Deconvoluted images were processed as TIFF files using the Image J software (FIG. 11).

### Immunohistochemistry (IHC)

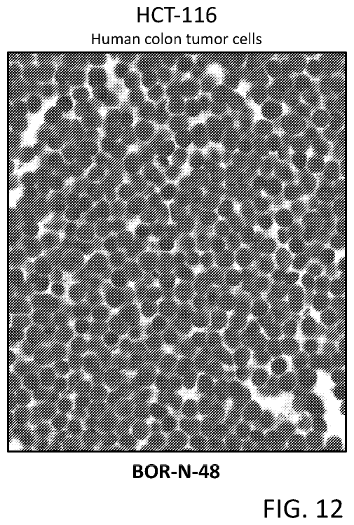

Human tumor cells (HCT-116) were grown to 80-90% confluency in T75 Flasks. 10-20M cells were collected in a 15-mL Falcon tube at 200 g for 5 min. The cell pellet was fixed in Formalin overnight. Next day, the formalin was gently replaced by 70% EtOH and pellets were embedded in HistoGel, and transferred to a biopsy cassette and stored briefly in 70% EtOH. Pellet blocks were cut and mounted on glass slides by The Human Tissue Resource Center at UChicago. Tissue sections were deparaffinized and rehydrated through xylenes and serial dilutions of EtOH to deionized water. They were incubated in antigen retrieval buffer (DAKO, S1699) and heated in steamer at over 97 degree C. for 20 minutes. Biotinylated anti-Borealin Fab was applied on tissue sections and incubated for 1-hour incubation at room temperature. Following TBS wash, the antigen-antibody binding was detected by Elite kit (PK-6100, Vector Laboratories) and DAB (DAKO, K3468) system. (FIG. 12).

All of the methods disclosed and claimed herein can be made and executed without undue experimentation in light of the present disclosure. While the compositions and methods of this invention have been described in terms of certain embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The references cited herein, to the extent that they provide exemplary procedural or other details supplementary to those set forth herein, are specifically incorporated herein by reference.
Winter, G., Griffiths, A. D., Hawkins, R. E. & Hoogenboom, H. R. (1994). Making antibodies by phage display technology. Annu. Rev. Immunol. 12, 433-455.
Boder, E. T. & Wittrup, K. D. (2000). Yeast surface display for directed evolution of protein expression, affinity, and stability. Methods Enzymol 328, 430-44.
Viti, F., Nilsson, F., Demartis, S., Huber, A. & Neri, D. (2000). Design and use of phage display libraries for the selection of antibodies and enzymes. Methods Enzymol 326, 480-505.
Bradbury, A. R., Sidhu, S., Dubel, S., and McCafferty, J. (2011) Beyond natural antibodies: the power of in vitro display technologies. Nat. Biotechnol. 29, 245-254
McCafferty, J., Griffiths, A. D., Winter, G. & Chiswell, D. J. (1990). Phage antibodies: filamentous phage displaying antibody variable domains. Nature 348, 552-554.
Kang, A. S., Barbas, C. F., Janda, K. D., Benkovic, S. J. & Lerner, R. A. (1991). Linkage of recognition and replication functions by assembling combinatorial antibody Fab libraries along phage surfaces. Proc. Natl. Acad. Sci. USA 88, 4363-4366.
Barbas, C. F., III, Kang, A. S., Lerner, R. A. & Benkovic, S. J. (1991). Assembly of combinatorial antibody libraries on phage surfaces: the gene III site. Proc. Natl. Acad. Sci. USA 88, 7978-7982.
Borrebaeck, C. A. K. E. (1992). Antibody engineering. A practical guide, W. H. Freeman & Co., New York.
Knappik, A., Ge, L., Honegger, A., Pack, P., Fischer, M., Wellnhofer, G., Hoess, A., Wolle, J., Pluckthun, A. & Virnekas, B. (2000). Fully synthetic human combinatorial antibody libraries (HuCAL) based on modular consensus frameworks and CDRs randomized with trinucleotides. J Mol Biol 296, 57-86.
Fellouse, F. A., Esaki, K., Birtalan, S., Raptis, D., Cancasci, V. J., Koide, A., Jhurani, P., Vasser, M., Wiesmann, C., Kossiakoff, A. A., Koide, S., and Sidhu, S. S. (2007) High-throughput generation of synthetic antibodies from highly functional minimalist phage-displayed libraries. J. Mol. Biol. 373, 924-940
Ura M, Mukherjee S, Marcon E, Koestler S A, Kossiakoff A A. Synthetic Antibodies Detect Distinct Cellular States of Chromosome Passenger Complex Proteins. J Mol Biol. 434, 167602 (2022).
Miller, K. R. et al. T cell receptor-like recognition of tumor in vivo by synthetic antibody fragment. PLoS One 7, e43746 (2012).
Paduch, M., Koide, A., Uysal, S., Rizk, S. S., Koide, S., and Kossiakoff, A. A. Generating conformation-specific synthetic antibodies to trap proteins in selected functional states. Methods 60, 3-14 (2013).
Mukherjee, S., Ura, M., Hoey, R. J., and Kossiakoff, A. A. A New Versatile Immobilization Tag Based on the Ultra High Affinity and Reversibility of the Calmodulin-Calmodulin Binding Peptide Interaction. J. Mol. Biol. 427, 2707-2725 (2015).

1. An antibody or antigen binding fragment comprising a heavy chain variable region and a light chain variable region, wherein the heavy chain variable region comprises a HCDR1, HCDR2, and HCDR3 having at least 80% sequence identity to the HCDR1, HCR2, HCR3 from a heavy chain variable region of a clone of Table 1 and wherein the light chain variable region comprises a LCDR1, LCDR2, and LCDR3 having at least 80% sequence identity to the LCDR1, LCDR2, and LCDR3 from the light chain variable region of the same clone of Table 1.

2. The antibody or antigen binding fragment of claim 1, wherein the heavy chain variable region comprises a HCDR1, HCDR2, and HCDR3 having the amino acid sequence of an of a HCDR1, HCDR2, and HCDR3 of a clone of Table 1 and wherein the light chain variable region comprises a LCDR1, LCDR2, and LCDR3 comprising the amino acid sequence of the LCDR1, LCDR2, and LCDR3 from the light chain variable region of the same clone of Table 1.

3. The antibody or antigen binding fragment of claim 1 or 2, wherein the HCDR1, HCDR2, HCDR2, LCDR1, LCDR2, and LCDR3 each comprise an amino acid sequence that has at least 80% sequence identity to an HCDR1, HCDR2, HCDR2, LCDR1, LCDR2, and LCDR3 of Table 1, wherein the HCDR1, HCDR2, HCDR2, LCDR1, LCDR2, and LCDR3 are from the same clone.

4. The antibody or antigen binding fragment of claim 1 or 2, wherein the HCDR1, HCDR2, HCDR2, LCDR1, LCDR2, and LCDR3 each comprise the amino acid sequence of an HCDR1, HCDR2, HCDR2, LCDR1, LCDR2, and LCDR3 of Table 1, wherein the HCDR1, HCDR2, HCDR2, LCDR1, LCDR2, and LCDR3 are from the same clone.

5. The antibody or antigen binding fragment of any one of claims 1-4, wherein the heavy chain variable region comprises an amino acid sequence with at least 80% sequence identity to a heavy chain variable region of a clone of Table 1 and/or the light chain variable region comprises an amino acid sequence with at least 80% sequence identity to the light chain variable region of the same clone of Table 1.

6. The antibody or antigen binding fragment of claim 5, wherein the heavy chain variable region comprises the amino acid sequence of a heavy chain variable region of a clone of Table 1 and/or the light chain variable region comprises the amino acid sequence of the same clone of Table 1.

7. The antibody or antigen binding fragment of any one of claims 1-6, wherein the antibody or antigen binding fragment comprises a heavy chain framework region (HFR) 1, HFR2, HFR3, and HFR4 and light chain framework region (LFR) 1, LFR2, LFR3, and LFR4, and wherein the HFR1, HFR2, HFR3, and HFR4 comprises an amino acid sequence with at least 80% sequence identity to an HFR1, HFR2, HFR3, and HFR4, respectively, of a clone of Table 1, and the LFR1, LFR2, LFR3, and LFR4 comprises an amino acid sequence with at least 80% sequence identity to the LFR1, LFR2, LFR3, and LFR4, respectively, of the same clone of Table 1.

8. The antibody or antigen binding fragment of any one of claims 1-6, wherein the HFR1, HFR2, HFR3, and HFR4 comprises the amino acid sequence of an HFR1, HFR2, HFR3, and HFR4, respectively, of a clone of Table 1, and the LFR1, LFR2, LFR3, and LFR4 comprises the amino acid sequence of the LFR1, LFR2, LFR3, and LFR4, respectively, of the same clone of Table 1.

9. The antibody or antigen binding fragment of any one of claims 1-8, wherein the antibody comprises a heavy chain and a light chain and wherein the heavy chain comprises an amino acid sequence with at least 70% sequence identity to a heavy chain of a clone of Table 1 and the light chain comprises an amino acid sequence with at least 70% sequence identity to the light chain of the same clone of Table 1.

10. The antibody or antigen binding fragment of claim 9, wherein the antibody comprises a heavy chain and a light chain and wherein the heavy chain comprises the amino acid sequence of a clone of Table 1 and the light chain comprises the amino acid sequence of the same clone of Table 1.

11. The antibody of any one of claims 1-10, wherein the antibody is human, chimeric, or humanized.

12. The antibody or antigen-binding fragment of any one of claims 1-11, wherein the antibody, or antigen binding fragment binds a Growth Hormone Receptor protein with a K_{D} of about 10⁻⁶ nM to about 10¹² pM.

13. The antibody or antigen binding fragment of any one of claims 1-12, wherein the antibody is a neutralizing antibody.

14. The antibody or antigen binding fragment of any one of claims 1-13, wherein the antibody is a human antibody, humanized antibody, recombinant antibody, chimeric antibody, an antibody derivative, a veneered antibody, a diabody, a monoclonal antibody, a single domain antibody, or a single chain antibody.

15. The antibody or antigen binding fragment of any one of claims 1-14, wherein the antibody or antigen binding fragment comprises a heavy chain framework region 1 (HFRW1), wherein the HFRW1 comprises an amino acid substitution with a D or T at the carboxy-terminal amino acid of the HFRW1.

16. The antigen binding fragment of any one of claims 1-15, wherein the antigen binding fragment is a single chain variable fragment (scFv), F(ab′)₂, Fab′, Fab, Fv, or rIgG.

17. A polypeptide comprising the antigen binding fragment of any one of claims 1-16.

18. The polypeptide of claim 17, wherein the polypeptide comprises at least two antigen binding fragments, wherein each antigen binding fragment is independently selected from an antigen binding fragment of any one of claims 1-16.

19. The polypeptide of claim 17 or 18, wherein the polypeptide is multivalent.

20. The polypeptide of any one of claims 17-19, wherein the polypeptide is bispecific.

21. The polypeptide of any one of claims 17-20, wherein the antigen binding fragment is a scFv or Fab.

22. The polypeptide of claim 21, wherein the Fab comprises the heavy and light chain of a Fab clone of Table 4.

23. The polypeptide of claim 21 or 22, wherein the VH is amino proximal to the VL.

24. The polypeptide of claim 21 or 22, wherein the VL is amino proximal to the VH.

25. The polypeptide of any one of claims 21-24, wherein VH region and VL region are separated by a peptide linker.

26. The polypeptide of claim 25, wherein the peptide linker is a glycine-serine linker.

27. The polypeptide of claim 25 or 26, wherein the peptide linker is at least 4 amino acids.

28. The polypeptide of any one of claims 21-27, wherein the polypeptide further comprises a second scFv or Fab.

29. The polypeptide of claim 28, wherein the second scFv or Fab specifically binds to CD3.

30. The polypeptide of any one of claims 21-27, wherein the polypeptide further comprises a CD3 binding region.

31. The polypeptide of claim 30, wherein the CD3 binding region comprises a scFv or Fab that specifically binds to CD3.

32. A chimeric antigen receptor (CAR) comprising:
a) an extracellular binding domain comprising the polypeptide of any one of claims 21-31;
b) a transmembrane domain; and,
c) a cytoplasmic region comprising a costimulatory domain and a primary intracellular signaling domain.

33. The CAR of claim 32, wherein the cytoplasmic region further comprises a co-stimulatory region between the transmembrane domain and the cytoplasmic region.

34. The CAR of claim 32 or 33, wherein the transmembrane domain comprises a transmembrane domain of CD28.

35. The CAR of any one of claims 32-34, wherein the primary intracellular signaling domain comprises a CD28 or CD3 zeta signaling domain.

36. The CAR of any one of claims 32-35, wherein the CAR comprises a peptide spacer between the extracellular binding domain and the transmembrane domain.

37. The CAR of claim 36, wherein the peptide spacer comprises the hinge region of an IgG molecule.

38. The CAR of claim 36 or 37, wherein the peptide spacer comprises the hinge and CH2CH3 region of an IgG molecule.

39. The CAR of any one of claim 32-38, wherein the CAR is multispecific.

40. The CAR of claim 39, wherein the CAR is bispecific.

41. A composition comprising the antibody or antigen binding fragment of any one of claims 1-16, the polypeptide of any one of claims 17-31, or the CAR of any one of claims 32-40.

42. The composition of claim 41, wherein the composition comprises a pharmaceutical excipient.

43. The composition of claim 41 or 42, wherein the composition further comprises an adjuvant.

44. The composition of any one of claims 41-43, wherein the composition is formulated for parenteral, intravenous, subcutaneous, intramuscular, or intranasal administration.

45. The composition of any one of claims 41-44, wherein the composition comprises at least two antibodies or antigen binding fragments.

46. One or more nucleic acids encoding the antibody or antigen binding fragment of any one of claims 1-16 or the polypeptide of any one of claims 17-31.

47. A nucleic acid encoding a heavy chain or light chain region, wherein the nucleic acid has at least 70% sequence identity to one of SEQ ID NOS:93-121.

48. A vector comprising the nucleic acid(s) of claim 46 or 47.

49. A host cell comprising the nucleic acid of claim 46 or 47, or the vector of claim 48.

50. The host cell of claim 49, wherein the host cell is an immune cell, a NK cell, a human cell, B cell, T cell, Chinese hamster ovary, NS0 murine myeloma cell, or PER.C6 cell.

51. The host cell of claim 50, wherein the immune cell is a T cell or NK cell and the engineered cell is an engineered T cell or an engineered NK cell.

52. The host cell of claim 51, wherein the T cell is a CD8⁺ T cell, CD4+ T cell, iNKT, or γδ T cell.

53. A method of a making a cell comprising transferring the nucleic acid(s) of claim 46 or 47 or the vector of claim 48 into a cell.

54. The method of claim 53, wherein the method further comprises culturing the cell under conditions that allow for expression of a polypeptide from the nucleic acid.

55. The method of claim 54, wherein the method further comprising isolating the expressed polypeptide.

56. The method of any one of claims 53-55, wherein the cell is a human cell, B cell, T cell, an immune cell, a NK cell, Chinese hamster ovary, NS0 murine myeloma cell, or PER.C6 cell.

57. The method of claim 56, wherein the immune cell is a T cell or NK cell and the engineered cell is an engineered T cell or an engineered NK cell.

58. The method of claim 57, wherein the T cell is a CD8⁺ T cell, CD4+ T cell, iNKT, or γδ T cell.

59. A method for producing a polypeptide comprising transferring the nucleic acid(s) of claim 46 or 47, or the vector of claim 48 into a cell and isolating polypeptides expressed from the nucleic acid.

60. The method of claim 59, wherein the cell is a human cell, B cell, T cell, Chinese hamster ovary, NS0 murine myeloma cell, or PER.C6 cell.

61. A method for treating or preventing cancer in a subject, the method comprising administering to the subject the antibody or antigen binding fragment of any one of claims 1-16, the polypeptide of any one of claims 17-31, the CAR of any one of claims 32-40, the composition of any one of claims 41-45, or the host cell of any one of claims 49-52.

62. The method of claim 61, wherein the subject is a human subject.

63. The method of claim 61 or 62, wherein the cancer is breast cancer, cervical cancer, prostate cancer, or leukemia.

64. The method of claim 61 or 62, wherein the subject has one or more symptoms of cancer.

65. The method of claim 61 or 62, wherein the subject does not have any symptoms of cancer.

66. The method of any one of claims 61-65, wherein the subject has been diagnosed with cancer.

67. The method of any one of claims 61-65, wherein the subject has not been diagnosed with cancer.

68. The method of any one of claims 61-67, wherein the subject has been previously treated for cancer.

69. The method of any one of claims 61-68, wherein the subject is administered an additional therapy.

70. The method of claim 69, wherein the additional therapy comprises radiotherapy, chemotherapy, or immunotherapy.

71. A method for evaluating a sample from a subject, the method comprising contacting a biological sample from the subject, or extract thereof, with at least one antibody, antigen binding fragment, or polypeptide of any one of claims 1-31.

72. The method of claim 71, wherein the at least one antibody, antigen binding fragment, or polypeptide is operatively linked to a detectable label.

73. The method of claim 71 or 72, wherein the method further comprises incubating the antibody, antigen binding fragment, or polypeptide under conditions that allow for the binding of the antibody, antigen binding fragment, or polypeptide to antigens in the biological sample or extract thereof.

74. The method of any one of claims 71-73, wherein the method further comprises detecting the binding of an antigen to the antibody, antigen binding fragment, or polypeptide.

75. The method of any one of claims 71-74, wherein the method further comprises contacting the biological sample with at least one capture antibody, antigen, or polypeptide.

76. The method of claim 75, wherein the at least one capture antibody, antigen binding fragment, or polypeptide comprises at least one antibody of claims 1-31.

77. The method of claim 75 or 76, wherein the capture antibody is linked to a solid support.

78. The method of any one of claims 71-77, wherein the biological sample comprises a tissue sample or a blood sample.

79. A method for diagnosing cancer in a subject, the method comprising contacting a biological sample from the subject, or extract thereof, with at least one antibody, antigen binding fragment, or polypeptide of any one of claims 1-31.

80. The method of claim 79, wherein the at least one antibody, antigen binding fragment, or polypeptide is operatively linked to a detectable label.

81. The method of claim 79 or 80, wherein the method further comprises incubating the antibody, antigen binding fragment, or polypeptide under conditions that allow for the binding of the antibody, antigen binding fragment, or polypeptide to antigens in the biological sample or extract thereof.

82. The method of any one of claims 79-81, wherein the method further comprises detecting the binding of an antigen to the antibody, antigen binding fragment, or polypeptide.

83. The method of any one of claims 79-82, wherein the method further comprises contacting the biological sample with at least one capture antibody, antigen binding fragment, or polypeptide.

84. The method of claim 83, wherein the at least one capture antibody, antigen, or polypeptide comprises at least one antibody, antigen binding fragment, or polypeptide of claims 1-31.

85. The method of claim 83 or 84, wherein the capture antibody is linked to a solid support.

86. The method of any one of claims 79-85, wherein the biological sample comprises a tissue sample or a blood sample.

87. A method for treating or preventing cancer in a subject, the method comprising administering to the subject an effective amount of a borealin-targeting agent.

88. The method of claim 87, wherein the borealin-targeting agent is an antibody or antibody fragment that specifically binds to borealin.

89. The method of claim 87, wherein the borealin-targeting agent is a chimeric antigen receptor comprising an antigen binding domain that specifically binds to borealin.
